(19) United States
(12) Patent Application Publication
(10) Pub. No.: US 2023/0152317 A1
(43) Pub. Date: May 18, 2023

(54) DIAGNOSIS AND TREATMENT OF COVID-19

(71) Applicant: LONDON HEALTH SCIENCES CENTRE RESEARCH INC., London (CA)

(72) Inventors: Douglas Fraser, London (CA); Gediminas Cepinskas, London (CA); Mark Daley, London (CA)

(73) Assignee: LONDON HEALTH SCIENCES CENTRE RESEARCH INC., London, ON (CA)

(21) Appl. No.: 17/919,360

(22) PCT Filed: Apr. 17, 2021
(86) PCT No.: PCT/CA2021/050526

§ 371 Date: Oct. 17, 2022

Related U.S. Application Data
(60) Provisional application No. 63/012,006, filed on Apr. 17, 2020.
(60) Provisional application No. 63/041,670, filed on Jun. 19, 2020.
(60) Provisional application No. 63/044,520, filed on Jun. 26, 2020.
(60) Provisional application No. 63/065,966, filed on Aug. 14, 2020.

(51) Int. Cl. G01N 33/569; A61K 31/737; A61K 31/727

(52) U.S. Cl. CPC G01N 33/56983; A61K 31/737; A61K 31/727; G01N 2333/165

(57) ABSTRACT

Methods of diagnosing COVID-19 infection in a subject comprising: (a) obtaining a test sample from the subject (b) comparing levels of a biomarker in the test sample with known normal reference levels of the biomarker, wherein an increase in the level f the biomarker in the test sample relative to the known reference levels of the biomarker is indicative of COVID-19 diagnosis in the subject, the biomarker being one or more of granzyme B, tumor necrosis factor (TNF), heat shock protein 70 (HSP70), interleukin-18 (IL-18), interferon-gamma-inducible protein 10 (IP-10) and elastase 2. These biomarkers are used as therapeutic targets for COVID-19 infection. Also, methods that serve to prognosticate the outcome, recovery and disease severity of COVID-19 patients.

### FIELD OF THE INVENTION

This invention relates to the diagnosis and treatment of COVID-19.

### BACKGROUND OF THE INVENTION

Throughout this application, various references are cited in brackets to describe more fully the state of the art to which this invention pertains. The disclosure of these references is hereby incorporated by reference into the present disclosure.

The Severe Acute Respiratory Syndrome Coronavirus-2 (SARS-CoV-2) is the virus that causes COVID-19 (or COVID19). COVID-19 primarily affects lungs, and in the most severe cases results in acute respiratory distress syndrome (ARDS) associated with or without multiorgan dysfunction (1-4). Once COVID-19 patients are admitted to the intensive care unit (ICU), the mortality rate is reported at 31% with a median of 9 days to ICU death (5). There are no specific therapies for COVID19, and patients are provided only supportive care. Identification of pathophysiological mediators, as well as prognostic biomarkers and/or therapeutic targets, is essential for improving COVID19 patient outcomes.

Recent reports and commentaries have suggested that the severity of COVID19 may be due to a “cytokine storm”, (6) which is the excessive or uncontrolled release of cytokines in response to a pathologic event, such as a viral infection. (7) These suggestions are due to increased inflammatory cytokine levels, such as interleukin 6 (IL6), as well as fever, cytopenia and hyperferritinemia. (8, 9)

Moreover, these commentaries have been accompanied by calls for the use of broad immunosuppression with steroids, intravenous immunoglobulin, and/or selective cytokine blockade as a therapeutic approach for COVID19. (7,10) While patient mortality could be improved with immunosuppressive therapies, the evidence for changes in specific cytokines is incomplete, and often observed at a single timepoint with limited comparison to control groups. (8, 9)

Additionally, as described in recent commentaries and reviews, the use of immunosuppressive therapies to treat critically ill patients, including those with ARDS, has often been challenging due to the potential to cause harm highlighting the need for rigorous data to support any proposed trials. (11,12)

It would be beneficial to have a small number of blood biomarkers measured that could serve not just to obtain an accurate diagnosis of COVID-19, but also serve to prognosticate the outcome and recovery of the patients. In addition, these biomarkers may identify and/or indicate potential therapeutic targets for COVID-19 infection.

### SUMMARY OF THE INVENTION

In one embodiment, the present invention is a method of diagnosing COVID-19 in a subject, the method comprising measuring levels of one or more metabolites listed in Table 13 in a sample taken from the subject, wherein a diagnosis of COVID-19 positive is indicated when the levels of said one or more metabolites are statistically different from known normal levels of said one or more metabolites.

In one embodiment of the method of diagnosing COVID-19, the one or more metabolites is one or more of kynurenine, arginine, sarcosine, lysoPC18:1, lysoPC20:4, lysoPC14:0, lysoPC17:0, lysoPC18:2, creatinine, creatine, C3OH, PC40:6AA, C5, C6:1, C3:1 and methylmalonic acid.

In another embodiment of the method of diagnosing COVID-19, the one or more metabolites is one more of kynurenine, arginine, sarcosine, lysoPC18:1, lysoPC20:4, lysoPC14:0, lysoPC17:0, and lysoPC18:2.

In another embodiment of the method of diagnosing COVID-19, the one or more metabolites is one more of creatinine, creatine, C3OH, PC40:6AA, C5, C6:1, C3:1 and methylmalonic acid.

In another embodiment of the method of diagnosing COVID-19, the one or more metabolites are kynurenine and arginine, and wherein the diagnosis of COVID-19 positive is indicated when a ratio of arginine/kynurenine levels is statistically decreased from known normal ratio levels of arginine/kynurenine.

In another embodiment of the method of diagnosing COVID-19, the one or more metabolite is kynurenine, and wherein the diagnosis of COVID-19 positive is indicated when the levels of kynurenine in the sample is statistically elevated from the known normal levels of kynurenine.

In another embodiment of the method of diagnosing COVID-19, the one or more metabolites is arginine, and wherein the diagnosis of COVID-19 positive is indicated when the levels of arginine in the sample is statistically decreased from the known normal levels of arginine.

In another embodiment of the method of diagnosing COVID-19, when the subject is indicated as being COVID-19 positive, the subject is treated with tryptophan, arginine, sarcosine and/or LysoPCs or any combinations thereof.

In another embodiment of the method of diagnosing COVID-19 in the subject, only when the subject is COVID-19 positive, the method further comprising treating the subject for COVID-19.

In another embodiment, the present invention relates to a method of determining COVID-19 disease severity for a COVID-19 patient, the method comprising, (a) measuring levels of one or more metabolites selected from Table 13 in a sample from the patient, (b) comparing the levels of the one or more metabolites to the known normal levels of said one or more metabolites, and based on the comparison, determining the severity of the disease.

In one embodiment of the method determining disease severity, the one or more metabolites is one or more of kynurenine, arginine, sarcosine, lysoPC18:1, lysoPC20:4, lysoPC14:0, lysoPC17:0, lysoPC18:2, creatinine, creatine, C3OH, PC40:6AA, C5, C6:1, C3:1 and methylmalonic acid.

In another embodiment of the method determining disease severity, the one or more metabolites is one more of kynurenine, arginine, sarcosine, lysoPC18:1, lysoPC20:4, lysoPC14:0, lysoPC17:0, and lysoPC18:2.

In another embodiment of the method determining disease severity, the one or more metabolites is one more of creatinine, creatine, C3OH, PC40:6AA, C5, C6:1, C3:1 and methylmalonic acid.

In another embodiment of the method determining disease severity, the one or more metabolites is creatinine and arginine.

In another embodiment of the method determining disease severity, the one or more metabolites is arginine.

In another embodiment of the method determining disease severity, the one or more metabolites is creatinine.

In another embodiment of the method determining disease severity, disease severity includes mortality risk.

In another embodiment of the method determining disease severity, the method further comprises treating the subject for COVID-19.

In another embodiment of the method determining disease severity, the method further comprises administering to the subject tryptophan, arginine, sarcosine and/or LysoPCs or any combinations thereof.

In another embodiment, the present invention is a method for the diagnosis of COVID-19 in a subject based on metabolomics analysis, said method comprising: (a) obtaining a metabolomics profile of biological samples collected from known COVID-19 positive patients and metabolomics profile of biological samples collected from known COVID-19 negative subjects to train a classification model and establish a COVID-19 positive class membership and a control class membership, and (b) analyzing an unknown biological sample collected from the subject to be diagnosed for COVID-19 and assigning a class membership for the unknown biological sample on the basis of the classification model established in step (a), wherein a diagnosis of COVID-19 positive is indicated when the unknown biological sample is assigned to the COVID-19 positive class membership.

In one embodiment of the method for the diagnosis of COVID-19 based on metabolomics analysis, the metabolomics profile includes the metabolites listed in table 13.

In another embodiment of the method for the diagnosis of COVID-19 based on metabolomics analysis, the metabolomics profile includes kynurenine, arginine, sarcosine, lysoPC18:1, lysoPC20:4, lysoPC14:0, lysoPC17:0 and lysoPC18:2.

In another embodiment of the method for the diagnosis of COVID-19 based on metabolomics analysis, the metabolomics profile includes kynurenine, arginine, sarcosine, lysoPC18:1, lysoPC20:4, lysoPC14:0, lysoPC17:0, lysoPC18:2, creatinine, creatine, C3OH, PC40:6AA, C5, C6:1, C3:1 and methylmalonic acid.

In another embodiment of the method for the diagnosis of COVID-19 based on metabolomics analysis, the metabolomics profile includes kynurenine and arginine.

In another embodiment of the method for the diagnosis of COVID-19 based on metabolomics analysis, the metabolomics profile include kynurenine.

In another embodiment of the method for the diagnosis of COVID-19 based on metabolomics analysis, only when the subject is assigned in the COVID-19 positive class membership, the method further comprising treating the subject for COVID-19.

In another embodiment of the method for the diagnosis of COVID-19 based on metabolomics analysis, only when the subject is assigned in the COVID-19 positive class membership, the subject is treated with tryptophan, arginine, sarcosine and/or LysoPCs or any combination thereof.

In another embodiment, the present invention provides for a COVID-19 diagnostic apparatus, the COVID-19 diagnostic apparatus including a computer readable storage medium and a computer program mechanism embedded therein, the computer program mechanism comprising executable instructions for performing a method of diagnosing for a COVID-19, said executable instructions comprising: (a) measuring levels of one or more metabolites listed in Table 13 in a sample taken from the subject, (b) providing a diagnosis of COVID-19 positive when the levels of said one or more metabolites are statistically different from known normal levels of said one or more metabolites.

In one embodiment of the COVID-19 diagnostic apparatus, the one or more metabolites is one or more of kynurenine, arginine, sarcosine, lysoPC18:1, lysoPC20:4, lysoPC14:0, lysoPC17:0, lysoPC18:2, creatinine, creatine, C3OH, PC40:6AA, C5, C6:1, C3:1 and methylmalonic acid.

In another embodiment of the COVID-19 diagnostic apparatus, the one or more metabolites is one more of kynurenine, arginine, sarcosine, lysoPC18:1, lysoPC20:4, lysoPC14:0, lysoPC17:0, and lysoPC18:2.

In another embodiment of the COVID-19 diagnostic apparatus, the one or more metabolites is one more of creatinine, creatine, C3OH, PC40:6AA, C5, C6:1, C3:1 and methylmalonic acid.

In another embodiment of the COVID-19 diagnostic apparatus, the one or more metabolites are kynurenine and arginine, and wherein the diagnosis of COVID-19 positive is indicated when a ratio of arginine/kynurenine levels is statistically decreased from known normal ratio levels of arginine/kynurenine.

In another embodiment of the COVID-19 diagnostic apparatus, the one or more metabolite is kynurenine, and wherein the diagnosis of COVID-19 positive is indicated when the levels of kynurenine in the sample is statistically elevated from the known normal levels of kynurenine.

In another embodiment of the COVID-19 diagnostic apparatus, the one or more metabolites is arginine, and wherein the diagnosis of COVID-19 positive is indicated when the levels of arginine in the sample is statistically decreased from the known normal levels of arginine.

In another embodiment of the COVID-19 diagnostic apparatus, only when the subject is COVID-19 positive, the instruction further includes providing an output for treating the subject for COVID-19

In another embodiment, the present invention provides for a kit for a COVID-19 diagnostic or quantitation assay, the kit comprising one or more internal standards suitable for mass spectrometry, packaging material, and instructions, wherein the one or more internal standards include the metabolites listed in FIGS. 1B, 2A and 3B.

In one embodiment of the kit, the one or more internal standards include one or a combination of kynurenine, arginine, lysophospholipds and creatinine.

In another embodiment of the kit, the one or more internal standards is labelled.

In another embodiment, the present invention relates to a method of treating COVID-19, the method comprising administering to a subject in need an effective amount of tryptophan, arginine, sarcosine and/or LysoPCs or any combinations thereof.

In one embodiment, the present invention is a method of treating COVID-19 in a patient, the method comprising administering to the patient an agent that reduces the levels of syndecan-1 degradation product in plasma or protects/restores vascular syndecan-1.

In one embodiment, the agent hat reduces the levels of syndecan-1 degradation product in plasma or protects/restores vascular syndecan-1 is sulodexide,

In another embodiment the agent hat reduces the levels of syndecan-1 degradation product in plasma or protects/restores vascular syndecan-1 is heparin/heparan. In one aspect the heparin is a low molecular weight heparin.

In another embodiment, the agent that reduces the levels of syndecan-1 degradation product in plasma or protects/restores vascular syndecan-1 is an inhibitor of a syndecan-1 sheddase.

In another aspect, the agent that reduces the levels of syndecan-1 degradation product in plasma or that protects and/or restores vascular syndecan-1 is a protease inhibitor, including a soybean-based protease inhibitor. In aspects, the protease inhibitor is an inhibitor of serine protease activity.

In another embodiment, the agent that reduces the levels of syndecan-1 degradation product in plasma or that protects and/or restores vascular syndecan-1 is an inhibitor of metalloproteinase (MMP) activity. In one aspect the MMP is MMP2, MMP7 or MMP9. In one aspect, the inhibitor of MMP activity is sphingosine-1-phosphate or a protease inhibitor, including a soybean-based protease inhibitor. In another aspect, the agent that reduces the levels of syndecan-1 degradation product in plasma or protects and/or restores vascular syndecan-1 is an inhibitor of granzyme B or an inhibitor of elastase 2. In one aspect, the inhibitor of granzyme B or the inhibitor of elastase 2 is a protease inhibitor, including soybean-based protease inhibitors.

In another embodiment, the agent that reduces the levels of syndecan-1 degradation product in plasma or protects/restores vascular syndecan-1 is a heparinase inhibitor.

In another embodiment, the patient is further treated with at least one additional agent. In one aspect, the at least one additional agent is an agent which blocks platelet aggregation or an anticoagulant or an agent which enhances thrombolysis or an agent which prevents glycocalyx degradation.

In another embodiment, the present invention is a method of diagnosing COVID-19 in a patient, the method comprising: (a) obtaining a test sample from the patient, (b) performing one or more assays configured to detect one or more biomarkers in the test sample, (c) obtaining the levels of the one or more biomarkers in the test sample, (c) comparing levels of the one or more biomarkers in the test sample with a normal control reference value of said one or more disease severity biomarkers, wherein an increase in the level of the one or more biomarkers in the test sample relative to the normal control reference value of said one or more biomarkers is indicative of COVID-19 diagnosis, wherein the one or more biomarkers are syndecan-1, hyaluronic acid (HA), chondroitin sulfate ADAMTS13, heparin sulfate, Protein C, soluble P-selectin (sP-selectin) and von Willebrand factor (vWF).

In another embodiment, the present invention is a method of predicting disease severity for a COVID-19 a patient, the method comprising: (a) obtaining a test sample from the patient, (b) performing one or more assays configured to detect one or more disease severity biomarkers in the test sample, (c) obtaining the levels of the one or more disease severity biomarkers in the test sample, (c) comparing levels of the one or more disease severity biomarkers in the test sample with a normal control reference value of said one or more disease severity biomarkers, wherein an increase in the level of the one or more disease severity biomarkers in the test sample relative to the normal control reference value of said one or more disease severity biomarkers is indicative of disease severity of the COVID-19 patient, wherein the one or more biomarkers are syndecan-1, HA, chondroitin sulfate ADAMTS13, heparin sulfate, Protein C, sP-selectin and vWF.

In another embodiment, this invention is a use of an agent that reduces the levels of syndecan-1 degradation product in plasma or protects/restores vascular syndecan-1 for the treatment of COVID-19.

In another embodiment, this invention is a use of an agent that reduces the levels of syndecan-1 degradation product in plasma or protects/restores vascular syndecan-1 in combination with at least one additional agent which blocks platelet aggregation, or an anticoagulant or which enhances thrombolysis for the treatment of COVID-19 or which prevents glycocalyx degradation.

In another embodiment, this invention is a use of an agent that reduces the levels of syndecan-1 degradation product in plasma or protects/restores vascular syndecan-1 for the preparation of a medicament for the treatment of COVID-19.

In one embodiment, the agent that reduces the levels of syndecan-1 degradation product in plasma or protects/restores vascular syndecan-1 is one or more of the agents cited in previous embodiments.

In one embodiment, the present invention is a method of predicting disease severity, including a mortality outcome for a COVID-19 patient with the method comprising: (a) obtaining a test sample from the patient, (b) performing one or more assays configured to detect one or more disease severity biomarkers in the test sample, (c) obtaining the levels of the one or more disease severity biomarkers in the test sample, (c) comparing levels of the one or more disease severity biomarkers in the test sample with a normal control (i.e. healthy) reference value of said one or more disease severity biomarkers, wherein an increase in the level of the one or more disease severity biomarkers in the test sample relative to the normal control reference value of said disease severity biomarker is indicative of disease severity, including mortality outcome, of the COVID-19 patient. The patient may be followed up to see if the one or more disease severity markers return to a normal level.

In one embodiment, step (c) comprises (i) measuring the expression levels of the one or more disease severity biomarkers in the test sample to form a set of raw expression data, (ii) normalizing the expression level for each of the one or more disease severity biomarkers, to form a set of normalized expression data, (iii) determining for the patient a risk of disease severity, including mortality, by comparing a divergence of the one or more disease severity biomarkers in the normalized expression data to reference expression data from the normal controls.

In another embodiment, the disease severity biomarker is selected from the analytes included in Table 4, 7 and 8. In one aspect, the disease severity marker is one or more analytes having an AUC of 0.7 or greater.

In another embodiment, the disease severity biomarker is one or more of HSP70, IL-IRA, IL 10, MIG, CLM-1, IL12RB1, CD83, FAM3B, IGF1R and OPTC.

In another embodiment, the present invention is a COVID-19 diagnostic apparatus, the COVID-19 diagnostic apparatus including a computer readable storage medium and a computer program mechanism embedded therein, the computer program mechanism comprising executable instructions for performing a method of predicting disease severity, including mortality outcome, for a COVID-19 patient, said executable instructions comprising: (a) comparing levels of one or more disease severity biomarker in a test sample of the subject, with known normal (i.e. healthy) reference levels of the one or more disease severity biomarker, and (b) providing a risk of mortality positive signal when there is an increase in the level of the biomarker the test sample relative to the known normal reference.

In one embodiment of the apparatus, the disease severity biomarker is selected from the analytes included in Table 4, 7 and 8.

In another embodiment of the apparatus, the disease severity biomarker is one or more of HSP70, IL-1RA, IL 10, MIG, CLM-1, IL12RB1, CD83, FAM3B, IGF1R and OPTC.

In another embodiment, the invention is a method for determining the likelihood that a COVID-19 patient is at risk of disease severity, including mortality, comprising: (a) measuring the patient's concentration of one or more markers listed in Table 4, 7 and 8 in absolute weight or absolute moles per volume; (b) comparing the measured concentration to a threshold level of concentration in absolute weight or absolute moles per volume corresponding to the measured one or more markers; and, (c) determining from the comparison the likelihood that the COVID-19 patient is at risk of mortality, wherein levels above the threshold level for the one or more markers concentration in absolute weight or absolute moles per volume indicate that the patient is at risk severe disease, including mortality.

In one embodiment, the present invention is a method of diagnosing COVID-19 infection in a subject. The method, in one embodiment, includes (a) obtaining a test sample from the subject (b) comparing levels of a biomarker in the test sample with a known reference value of said biomarker, wherein an increase in the level of the biomarker in the test sample relative to the known reference value of said biomarker is indicative of positive COVID-19 diagnosis in the subject. In one aspect, the biomarker is one or more of granzyme B, TNF, HSP70 and IL18. In another aspect the biomarker is one or more of granzyme B, TNF, HSP70 IL18, interferon-gamma-inducible protein 10 (IP-10) and elastase 2. In one aspect the levels are obtained using quantitative measurements. In another aspect the known reference value of the biomarker is a known normal reference. In another aspect the known reference value of the biomarker is a known abnormal reference of COVID-19 negative (COVID-19−) subjects. In another aspect the known reference value of the biomarker is the level of said biomarker obtained from a normal sample. In another aspect, the known reference value of the biomarker is the level of the biomarker from an abnormal sample.

In one embodiment according to the previous embodiment, the method further comprises (c) treating the subject for COVID-19 infection when the level of the biomarker in the test sample is increased relative to the normal or abnormal control sample with an inhibitor or antagonist of the biomarker.

In one embodiment of the method of diagnosing COVID-19 of the present invention, the method further includes obtaining a sample from the subject during the subject's recovery for COVID-19 (i.e. during the subject's rehabilitation therapy), wherein decrease in the levels of the biomarker in the recovery sample relative to the levels obtained in the test sample is indicative of a normalization of the subject.

In another embodiment, the present invention is a method for the diagnosis of COVID-19 in a subject comprising (a) comparing the proteomic profile of a test sample of a biological fluid of the subject with a control proteomic profile, wherein the control proteomic profile is a normal sample, an abnormal sample, a normal reference proteomic profile or an abnormal reference proteomic profile comprising at least one protein biomarker of the present invention; and (b) diagnosing said subject with COVID-19 if the proteomic profile of the test sample shows a unique expression of the at least one protein biomarker; wherein said at least one protein biomarker is granzyme B, TNF, Hsp 70, IL-18, IP-10 and elastase 2, and wherein said test sample proteomic profile and said control proteomic profile comprise information of the expression of one or more of granzyme B, TNF, Hsp 70, IL-18, IP-10 and elastase 2.

In another embodiment, the present invention is a method of treating COVID-19 infection in a subject, the method comprising administering to the subject an inhibitor or antagonist of granzyme B. In one aspect, the inhibitor or antagonist of granzyme B is a protease inhibitor. In another aspect the protease inhibitor is a soybean-based protease inhibitor. Protease inhibitors include Kunitz-type protease inhibitor and Bowman-Birk type protease inhibitors.

In another embodiment, the present invention is a method of treating COVID-19 infection in a subject, the method comprising administering to the subject an inhibitor or antagonist of TNF.

In another embodiment, the present invention is a method of treating COVID-19 infection in a subject, the method comprising administering to the subject an inhibitor or antagonist of HSP70.

In another embodiment, the present invention is a method of treating COVID-19 infection in a subject, the method comprising administering to the subject an inhibitor or antagonist of IL-18.

In another embodiment, the present invention is a method of treating COVID-19 infection in a subject, the method comprising administering to the subject an inhibitor or antagonist of IP-10.

In another embodiment, the present invention is a method of treating COVID-19 infection in a subject, the method comprising administering to the subject an inhibitor or antagonist of elastase 2.

In another embodiment, the present invention is a method of treating COVID-19 infection in a subject, the method comprising administering to the subject an inhibitor or antagonist of IL-10.

In another embodiment, the present invention is a method of treating COVID-19 infection in a subject, the method comprising administering to the subject an inhibitor or antagonist of elastase 2. In one aspect, the inhibitor or antagonist of elastase 2 is a protease inhibitor. In another aspect the protease inhibitor is a soybean-based protease inhibitor.

In another embodiment, the present invention is a use of the level of a biomarker in the diagnosis of COVID-19, wherein the biomarker is one or more of granzyme B, TNF, HSP70, IL-18, IP-10 and elastase 2. In one aspect the biomarker is one or more of granzyme B, TNF, HSP70 and IL-18.

In another embodiment, the present invention is a use of a granzyme B inhibitor or antagonist in the treatment of COVID-19. In one aspect, the granzyme B inhibitor or antagonist is a protease inhibitor. In another aspect the protease inhibitor is soybean-based.

In another embodiment, the present invention is a use of a TNF inhibitor or antagonist in the treatment of COVID-19.

In another embodiment, the present invention is a use of a HSP 70 inhibitor or antagonist in the treatment of COVID-19.

In another embodiment, the present invention is an IL-18 inhibitor or antagonist in the treatment of COVID-19.

In another embodiment, the present invention is an IP-10 inhibitor or antagonist in the treatment of COVID-19.

In another embodiment, the present invention is a use of an elastase 2 inhibitor or antagonist in the treatment of COVID-19. In one aspect, the elastase-2 inhibitor or antagonist is a protease inhibitor. In another aspect the protease inhibitor is soybean-based.

In another embodiment, the present invention is a sue of a protease inhibitor in the treatment of COVID-19. In aspects, the protease inhibitor is a soybean-based protease inhibitor.

In another embodiment, the present invention is a COVID-19 diagnostic apparatus, the COVID-19 diagnostic apparatus including a computer readable storage medium and a computer program mechanism embedded therein, the computer program mechanism comprising executable instructions for performing a method of diagnosing COVID-19 in a subject, said executable instructions comprising: (a) comparing levels of a biomarker in a test sample of the subject, with known reference levels of the biomarker, and (b) providing a COVID-19 positive signal when there is an increase in the level of the biomarker the test sample relative to the control sample, the biomarker being one or more of granzyme B, TNF, HSP70, IL-18, IP-10 and elastase 2. In one aspect of this embodiment the known reference levels of the biomarker is a known abnormal level of the biomarker in a COVID-19− subject. In another aspect of this embodiment the known reference levels of the biomarker is a level of the biomarker in a normal subject.

In one embodiment of the COVID-19 diagnostic apparatus of the present invention, the biomarker is one or more of granzyme B, TNF, HSP70, IL18, IP10 and elastase 2.

In another embodiment of the COVID-19 diagnostic apparatus of the present invention, the instructions further include comparing the levels of the biomarker in the test sample, with the levels of the biomarker in a sample obtained from the subject during the subject's treatment of COVID-19, wherein a decrease in the level of biomarker during the treatment relative to the levels of the biomarker in the test ample is indicative of a normalization of the subject.

### BRIEF DESCRIPTION OF THE DRAWINGS

The following figures illustrate various aspects and preferred and alternative embodiments of the invention.

FIGS. 1A-1B: (A) Subjects plotted in two dimensions following dimensionality reduction of their respective metabolites by stochastic neighbor embedding. Green circled dots represent healthy control subjects, while orange dots represent age- and sex-matched COVID-19+ ICU patients (ICU day 1 plasma). The dimensionality reduction shows that based on the plasma metabolites the two cohorts are distinct and easily separable. The axes are dimension-less. (B) ROC analysis of healthy control subjects versus COVID-19+ patients, using an Arginine/Kynurenine ratio, demonstrates an AUC of 1.00 (P=0.0002). The cutoff value is 15.6. The diagonal broken blue line represents chance (AUC 0.50).

FIGS. 2A-2B: (A) ROC analysis of COVID-19+ versus COVID-19− ICU patients, using an Arginine/Kynurenine ratio, demonstrates an AUC of 0.98 (P=0.005). The diagonal broken blue line represents chance (AUC 0.50). (B) A time plot demonstrating the Arginine/Kynurenine ratio for both COVID-19+ (orange dots) and COVID-19− (circled blue dots) patients over 10 ICU days. The two cohorts are significantly different on ICU days 1 and 3 (*** P=0.005). Healthy control range values are represented by green shading.

FIG. 3A-3B (A) COVID-19+ ICU patients plotted in two dimensions following dimensionality reduction of their respective metabolites by stochastic neighbor embedding. Blue (not circled) dots represent COVID-19+ ICU patients that survived their ICU stay, while circled orange dots represent COVID-19+ ICU patients that died (ICU day 1 plasma). The dimensionality reduction shows that based on the plasma metabolites the two cohorts are distinct and easily separable. The axes are dimension-less. (B) A time plot demonstrating the Creatinine/Arginine ratio for COVID-19+ ICU patients over 10 ICU days that either survived (blue dots) or died (circled orange dots). The two cohorts are significantly different on ICU days 1 and 3 (** P=0.01). Healthy control range values are represented by green shading.

FIGS. 4A to 4B. (4A) Subjects plotted in two dimensions following dimensionality reduction by stochastic neighbor embedding. Red dots represent COVID-19+ subjects (n=10, days 1-3) and circled green dots healthy control subjects (n=10). The dimensionality reduction shows that based on daily thrombotic factor and endothelial injury marker concentrations, the two cohorts are distinct and easily separable. The axes are dimension-less. (4B) ICU sepsis patients plotted in two dimensions following dimensionality reduction by stochastic neighbor embedding. Red dots represent COVID-19+ subjects (n=10, days 1-3) and circled green dots represent COVID-19− subjects (n=10, days 1-3). The dimensionality reduction shows that based on daily thrombotic factor and endothelial injury marker concentrations, the two cohorts are distinct and easily separable. The axes are dimension-less.

FIG. 5: Time course for 3 endothelial injury markers between COVID-19+ and COVID-19− ICU patients. sP-selectin, hyaluronic acid and syndecan-1 remained elevated until the final plasma measurements on ICU day 7. Daily values are represented as means (±SEM; *P<0.05).

FIG. 6 illustrates basal nitric oxide in human hPMVEC untreated (control “−Hyaluronidase′) and treated with hyaluronidase (+Huyaluronidase). Hyaluronidase treatment decreased basal intracellular nitric oxide production by 98% to 64±87.5.

FIG. 7: The COVID-19 case patient and 20 healthy control subjects plotted in two dimensions following dimensionality reduction by stochastic neighbor embedding. The Purple dot represents the COVID-19 patient, while the circled yellow dots represent the healthy controls. The dimensionality reduction shows that based on 59 plasma analyte concentrations, the COVID-19 patients is distinct and easily separable. The axes are dimension-less.

FIG. 8. Subjects plotted in two dimensions following dimensionality reduction by stochastic neighbor embedding. Purple dots represent COVID-19+ subjects, yellow dots (circled) healthy controls. The dimensionality reduction shows that based on daily plasma analyte concentrations, the two cohorts are distinct and easily separable. The axes are dimension-less.

FIG. 9. Subjects plotted in two dimensions following dimensionality reduction by stochastic neighbor embedding. Purple dots represent COVID-19+ subjects, yellow dots (circled) COVID-19−. The dimensionality reduction shows that based on daily plasma analyte concentrations, the two cohorts are distinct and easily separable. The axes are dimension-less.

FIG. 10A. tSNE plot demonstrating that the proteome between COVID-19+ patients on ICU day 1 that either survived or died are distinct and easily separable (circled dots patients that survived, non-circled dots patients that died).

FIG. 10B. tSNE plot demonstrating that the proteome between COVID-19+ patients on ICU day 3 that either survived or died are distinct and easily separable (circled dots patients that survived, non-circled dots patients that died).

FIGS. 11A-11F. Time course for the top 6 inflammatory analytes between COVID-19+ and COVID-19− ICU patients. Daily values are represented as means (±SEM). *p<0.01. 11A: CLM-1; 11B: IL12RB1; 11C: CD83; 11D: FAM3B; 11E: IGF1R 3.8, and 11F: OPTC.

FIG. 12. Subjects plotted in two dimensions following dimensionality reduction by stochastic neighbor embedding. Purple (dark) dots represent coronavirus disease 2019 positive (COVID-19+) subjects, yellow (light) dots COVID-19−. The dimensionality reduction shows that based on daily plasma analyte concentrations, the two cohorts are distinct and easily separable. The axes are dimension less.

FIGS. 13A-F: Time course for the top six inflammatory analytes between COVID-19+ and COVID-19− ICU patients. Daily values are represented as mean (±sem). *p<0.01. 13A: TNF; 13B: Granzyme B; 13C: HSP70; 13D: IL18: 13E: IP10; 13F: Elastase.

FIGS. 14A to 14J: Time course for inflammatory analytes between COVID-19+ and COVID-19− ICU patients. 14A) IL10; 14B) MIG; 14C) M-CSF; 14D) IFNγ; 14E) IL8; 14F) MMP8; 14G) IL2; 13H) IL15; 14I) IL01RA; 14J) MMP1. Daily values are represented as means (±SEM). *p<0.01.

### DESCRIPTION OF THE INVENTION

### Abbreviations

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The contents of all documents (including patent documents and non-patent literature) cited in this application are incorporated herein by reference.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“COVID-19− subjects” or “COVID-19 negative subjects” are subjects who are septic with Acute lung injury (ALI), but are confirmed SARS-CoV-2 negative.

“COVID-19+ subjects” (or patients) or “COVID-19 subjects” are subjects who are septic with Acute lung injury (ALI) and positive for SARS-CoV-2.

“Metabolome” refers to the collection of all metabolites in a biological cell, tissue, organ or organism, which are the end products of cellular processes. “Metabolome” includes lipidome, sugars, nucleotides and amino acids.

“Metabolomic profiling” refers to the characterization and/or measurement of the small molecule metabolites in biological specimen or sample, including cells, tissue, organs, organisms, or any derivative fraction thereof and fluids such as blood, blood plasma, blood serum, capillary blood, venous blood, saliva, synovial fluid, spinal fluids, urine, bronchoalveolar lavage, tissue extracts, tears, volatile organic compounds (VOCs), breath samples, sweat and so forth. This characterization may be targeted (limited to a defined number of specific compounds) or untargeted/nontargeted in nature (not limited to a defined or known number of compounds).

The metabolite profile may include information such as the quantity and/or type of small molecules present in the sample. The ordinarily skilled artisan would know that the information which is necessary and/or sufficient will vary depending on the intended use of the “metabolite profile.” For example, the “metabolite profile,” can be determined using a single technique for an intended use but may require the use of several different techniques for another intended use depending on such factors as the disease state involved, the types of small molecules present in a particular targeted cellular compartment, the cellular compartment being assayed per se., and so forth.

The relevant information in a “metabolite profile” may also vary depending on the intended use of the compiled information, e.g., spectrum. For example, for some intended uses, the amounts of a particular metabolite or a particular class of metabolite may be relevant, but for other uses the distribution of types of metabolites may be relevant.

Metabolite profiles may be generated by several methods, e.g., HPLC, thin layer chromatography (TLC), electrochemical analysis, Mass Spectroscopy (MS), refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), fluorescence spectroscopy, dual polarisation interferometry, computational methods, liquid chromatography (LC) Light Scattering analysis (LS), gas chromatography (GC), or GC coupled with MS, direct injection (DI) coupled with LC-MS/MS and/or other methods or combination of methods known in the art.

The term “subject” as used herein refers all members of the animal kingdom including mammals, preferably humans.

The term “patient” as used herein refers to a subject that has or is suspected of having COVID-19.

The terms “test sample” or “sample” include biological specimen, including cells, tissue, organs, organisms, or any derivative fraction thereof and fluids such as blood, blood plasma, blood serum, capillary blood, venous blood, saliva, synovial fluid, spinal fluids, urine, bronchoalveolar lavage, tissue extracts, tears, volatile organic compounds (VOCs), breath samples, sweat and so forth.

“Plasma” is the clear, straw-colored liquid portion of blood that remains after red blood cells, white blood cells, platelets and other cellular components are removed.

The term “proteome” is used herein to describe a significant portion of proteins in a biological sample at a given time. The concept of proteome is fundamentally different from the genome. While the genome is virtually static, the proteome continually changes in response to internal and external events.

The term “proteomic profile” is used to refer to a representation of the expression pattern of a plurality of proteins in a biological sample, e.g., a biological fluid at a given time. The proteomic profile can, for example, be represented as a mass spectrum, but other representations based on any physicochemical or biochemical properties of the proteins are also included. Thus, the proteomic profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel. Proteins can be measured with antibody tests (i.e. Western blotting, Luminex bead-based assays, Proximity Extension Assay (PEA), planar multiplex assays, electrochemiluminescence, proximal extension assay with oligonucleotide-labeled antibodies, ELISA and RIA), flow cytometry or mass spec techniques. Enzymes can be measured with enzyme assays that measure either the consumption of a substrate or production of product over time. Differential expression profiles may have important diagnostic value, even in the absence of specifically identified proteins. Single protein spots can then be detected, for example, by immunoblotting, multiple spots or proteins using protein microarrays. The proteomic profile typically represents or contains information that could range from a few peaks to a complex profile representing 50, 1,000 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50 proteins, or over 1,000 proteins.

“Disease severity” is used in this document to characterize the impact that a disease process has on the utilization of resources, comorbidities, and mortality. Disease severity in COVID-19 patients show sequential organ dysfunction, requirement of higher or greater levels of support and morbidity.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington:

The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient”, “active compound”, and “active substance” refer to a compound, which is administered, alone or in combination with one or more pharmaceutically acceptable excipients or carriers, to a subject for treating, preventing, or ameliorating one or more symptoms of COVID-19 pathology.

The terms “agent”, “drug”, “therapeutic agent”, and “chemotherapeutic agent” refer to a compound, or a pharmaceutical composition thereof, which is administered to a subject for treating, preventing, or ameliorating one or more symptoms of COVID-19 pathology.

By “inhibitor” is meant any molecule that inhibits, suppresses or causes the cessation of at least one biological activity of a biomarker of the present invention, e.g. by reducing, interfering with, blocking, or otherwise preventing the interaction or binding of the biomarker to its natural target. Inhibitors include low molecular weight antagonists, antibodies, proteins, peptides or ligands that impair the biological action of the biomarker, antisense oligonucleotides, including anti-sense RNA molecules and anti-sense DNA molecules that are complimentary to a nucleic acid sequence from a gene or genes that encode the biomarker may be used in the methods of the present invention to block the translation of mRNA and inhibit protein synthesis, or increasing mRNA degradation, thus decreasing the level of biomarker protein, and thus activity, in a cell. Small inhibitory RNA (siRNA) is a form of gene silencing triggered by double-stranded RNA (dsRNA). In siRNA sequence-specific, post-transcriptional gene silencing in animals and plants may be initiated by double-stranded RNA (dsRNA) that is homologous in sequence to the silenced gene. A siRNA (small interfering RNA) is designed to target and thus to degrade a desired mRNA (in this case encoding mRNA of a suitable biomarker of the present invention) in order not to express the encoded protein.

Ribozymes may also function as inhibitors of protein expression for use in the present invention. Ribozymes are enzymatic RNA molecules capable of catalyzing the specific cleavage of RNA.

The compositions of the present invention include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The compositions may conveniently be presented in unit dosage form and may be prepared by any of the methods well known in the art of pharmacy.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

The amount of active ingredient that may be combined with the carrier materials to produce a single dosage form will vary depending upon the host treated and the particular mode of administration.

The compounds can be administered in various modes, e.g., orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

### Overview

The present invention relates to the diagnosis, assessing disease severity and treatment of COVID-19 patients and follow-up the recovery of the patients.

Diagnosis

In one embodiment, a method of diagnosing COVID-19 infection in a patient includes comparing the levels of a single biomarker or a cohort of biomarkers (i.e. one or more biomarkers) in a subject's sample using quantitative or non-quantitative measurements of said biomarker or cohort of biomarkers to the levels of said single biomarker or cohort of biomarker in a known normal reference range, or in a normal population. In the case of non-quantitative measurements, the levels of the one or more biomarkers can be normalized and compared by reference to a known reference value. A change, an increase or decrease, in the level of the single biomarker or cohort of biomarkers in the subject's sample relative to the normal reference range being indicative of the subject having COVID-19 infection.

A method of diagnosing COVID-19 infection in a patient includes: (a) obtaining a test sample from the patient, (b) performing one or more assays configured to detect one or more biomarkers in the test sample, (c) obtaining the levels of the one or more biomarkers in the test sample, (c) comparing levels of the one or more biomarkers in the test sample with a normal control (i.e. healthy) reference value of said one or more disease severity biomarkers, wherein an increase or decrease in the level of the one or more biomarkers in the test sample relative to the normal control reference value of said one or more biomarkers is indicative of positive COVID-19 diagnosis.

In one embodiment, the one or more biomarkers are those listed in Tables 2 and 3, and an increase in the levels of the one or more biomarkers listed in Table 2 and 3 relative to a normal control being indicative of positive COVID-19 diagnosis.

In one embodiment of the present invention, the one or more biomarkers are granzyme B, TNF, HSP 70, IL 18, IL 10 and elastase 2, and an increase in the levels of any one of granzyme B, TNF, HSP 70, IL 18, IL 10 and elastase 2 relative to the normal control being indicative of positive COVID-19 diagnosis.

In another embodiment, the one or more biomarkers are granzyme B, TNF, HSP 70 and IL 18.

In another embodiment, the one or more biomarkers are syndican-1, hyaluronic acid (HA), chondroitin sulfate ADAMTS13, heparin sulfate, Protein C, sP-selectin and von Willebrand factor (vWF).

In another embodiment, the method of diagnosing COVID-19 in a subject comprises (a) measuring an amount of one or more metabolites in a sample from the subject, (b) determining a parameter from the amount of each of the one or more metabolites, (c) comparing the parameter to one or more cutoff values, and based on the comparison, determining whether the subject is COVID-19 positive. For example, an increase in the levels of kynurenine in COVID-19 positive patients relative to healthy control subjects, or a decrease in lysoPCs in COVID-19 positive patients relative to healthy control subjects or a decrease in arginine levels in COVID-19 patients relative to healthy controls. The changes in kynurenine, lysoPCs and arginine, taken alone or in any combination thereof (i.e. levels of kynurenine alone, levels of lysoPCs alone, levels of arginine alone, levels of kynurenine and lysoPCs, levels of kynurenine and arginine, levels of arginine and lysoPCs) can be used to discriminate between COVDID-19 positive patients and healthy control subjects.

In one embodiment, the one or more diagnostic biomarkers are those listed in Table 13. In another embodiment, the one or more biomarkers are those listed in Tables 14, 15 and 16. In another embodiment, the one or more biomarkers are those listed in Table 14. In another embodiment, the one or more biomarkers are those listed in Table 15. In another embodiment the one or more biomarkers are those listed in Table 16. In another embodiment, the one or more biomarkers of the present disclosure are arginine, kynurenine, sarcosine, lysophosphatidylcholines and creatinine. In another embodiment the biomarker is kynurenine and/or arginine. In another embodiment the biomarker is one or more lysophosphatidylcholines, kynurenine and/or arginine In another embodiment, the biomarker is arginine. In another embodiment, the biomarker is kynurenine. In another embodiment, the biomarker is creatinine. In another embodiment, the biomarker is sarcosine. In another embodiment, the biomarker is creatinine. In another embodiment, the biomarker is one or more lysophosphatidylcholines.

In embodiments, the one or more metabolites is one or more of kynurenine, arginine, sarcosine, lysoPC18:1, lysoPC20:4, lysoPC14:0, lysoPC17:0, lysoPC18:2, creatinine, creatine, C3OH, PC40:6AA, C5, C6:1, C3:1 and methylmalonic acid, or the one or more metabolites is one more of kynurenine, arginine, sarcosine, lysoPC18:1, lysoPC20:4, lysoPC14:0, lysoPC17:0, and lysoPC18:2, or the one or more metabolites is one more of creatinine, creatine, C3OH, PC40:6AA, C5, C6:1, C3:1 and methylmalonic acid, or the one or more metabolites are kynurenine and arginine, and the parameter is an arginine/kynurenine ratio. The cutoff value is equal or larger than 11.6. The cutoff value is equal or larger than 15.7.

The methods and computer programs of the present invention may be used in point-of-care metabolomics testing with portable, table/counter-top or hand-held instruments that generate metabolite profiles.

The diagnostic methods may be used during the treatment of a COVID-19 patient. Returns to a normal level of the biomarkers may serve as an aid in following medical interventions of individuals affected by COVID-19.

When following the diagnosis, and a subject is COVID-19 positive, the method further comprising treating the subject for COVID-19 with any known method for treating COVID-19 or with a method of the present invention.

Assessing Disease Severity

In embodiments the present invention relates to biomarkers measured that serve to assess or predict the severity of the illness and mortality for COVID-19 patients, particularly for patients in ICUs, shortly after their admissions.

Biomarkers of the present invention also serve as predictors of disease severity, including mortality in COVID-19 patients. In embodiments, the one or more biomarkers are those listed in Table 7, Table 8 and Table 4. In other embodiments, the one or more biomarkers is one or more of HSP70, IL-1RA, IL10, MIG, CLM-1, IL12RB1, CD83, FAM3B, IGF1R and OPTC. In embodiments, the one or more biomarkers are syndican-1, HA, chondroitin sulfate ADAMTS13, heparin sulfate, Protein C, sP-selectin and vWF.

In one embodiment, the present invention involves comparing the levels of a single biomarker or a cohort of biomarkers in a subject's sample, using quantitative or non-quantitative measurements of said biomarker or cohort of biomarkers to the levels of said single biomarker or cohort of biomarker in a known normal reference range, or in a normal population. In the case of non-quantitative measurements, the levels of the one or more biomarkers can be normalized and compared by reference to a known reference value. An increase in the level of the one or more biomarker in the subject's sample relative to the known reference values for the one or more biomarker in a normal healthy control being indicative of disease severity, including mortality outcome. Blood is taken from a COVID-19 patient and analytes are measured in a sample taken from the patient. The analytes are compared to a known reference value of cutoff value established in COVID-19 negative controls. An increase in the measurements, such as absolute concentrations (absolute weight, absolute moles per volume, etc.), of the analytes in the sample of the patient is indicative of COVID-19 disease severity or risk of death of the patient.

Since metabolites exist in a very broad range of concentrations and exhibit chemical diversity, there is no one instrument that can reliably measure all of the metabolites in the non-human or human metabolome in a single analysis. Instead, practitioners of metabolomic profiling generally use a suite of instruments, most often involving different combinations of liquid chromatography (LC) or gas chromatography (GC) coupled with MS, to obtain broad metabolic coverage [Circulation. 2012; 126: 1110-1120] Other instruments such as electrochemical analysis, RI, UV, near-IR, LS, GC and so forth may also be used.

Point-of-care testing (e.g., hand-held or table-top antibody testing, lateral flow device, chip or MS) could be developed to identify COVID-19 patients, and to prognosticate outcome and/or stratify to treatment.

A library of the measurements of the biomarkers of the present invention may be established for diagnosed COVID-19 cases. This library may be used as the predetermined, control set of biomarker measurements of COVID-19. Similarly, a predetermined set of normal biomarker measurements may be obtained from subjects known not to have COVID-19. A comparison may be made of the patient's biomarker's measurements, the predetermined biomarker measurements of COVID-19 and the predetermined biomarker measurements of normal or control samples to determine not only if the patient has COVID-19 but also the prognosis.

The libraries of predetermined biomarker measurements may be provided in a computer product (memory sticks, as an app for hand-held devices such as pads and cellular phones and so forth), or they may be uploaded to the memory of a computer system, including main frames, desk-tops, lab tops, hand-held devices such as pads and cellular phones. Blood or any other bodily fluid, for example whole blood, blood plasma, blood serum, capillary blood, venous blood, saliva, synovial fluid, urine, spinal fluid, bronchoalveolar lavage, tears, volatile organic compounds (VOCs), breath samples sweat, extracts and so forth, may be taken from a patient. Biomarker measurements may be obtained from the patient's sample using any known technology (for example, high performance liquid chromatography, thin layer chromatography, electrochemical analysis, mass spectroscopy (MS), refractive index spectroscopy, ultra-violet spectroscopy, fluorescent analysis, radiochemical analysis, near-infrared spectroscopy, light scattering analysis, gas chromatography (GC), or GC coupled with MS, direct injection (DI) coupled with LC-MS/MS and so forth) or antibody tests (i.e. Western blotting, Luminex bead-based assays, Proximity Extension Assay (PEA), planar multiplex assays, electrochemiluminescence, proximal extension assay with oligonucleotide-labeled antibodies, ELISA and RIA). The patient's biomarker measurements may then be uploaded to the computer system (main frames, desk-tops, lab tops, hand-held devices and so forth). An operator may then compare the patient's biomarker measurements with the predetermined set of biomarker measurements of COVID-19 and/or the predetermined biomarker measurements of a control or normal to determine not only if the patient has COVID-19, but also the prognosis, or whether a treatment is efficient.

Treatment

The present invention relates also to the treatment of COVID-19 patients.

In embodiments, the methods of the present invention comprise administering to a patient in need an effective amount of an agent that reduces the levels of syndecan-1 degradation product in plasma or protects and/or restores vascular syndecan-1. In embodiments, the methods of the present invention involve administering to the patient an effective amount of an agent that inhibits syndican-1 shedding. In embodiments, examples of the agent include sulodexide (inhibitor of platelet aggregation/reconstruction of glycocalyx) and inhibitors of metalloproteinase (MMP) activity such as sphingosine-1-phosphate or a protease inhibitor. In embodiments, the agent that reduces the levels of syndecan-1 degradation product or protects and/or restores vascular syndecan-1 is protease inhibitor, including soybean-based protease inhibitor. Soybean-based protease inhibitors inhibit the activity of MMPs, granzyme B and/or elastase-2 that can also cleave the glycocalyx. The compounds disclosed herein may also be combined or used in combination with other agents useful in the treatment of COVID-19.

It has now been found that the administration of pharmaceutical compositions containing therapeutically effective amounts of an agent that reduces the levels of syndican-1 in plasma, or protects/restores vascular syndecan-1, can be used to treat COVID-19 positive (+) patients, particularly COVID-19+ patients suffering thrombosis.

Sulodexide is a glycosaminoglycan of natural origin extracted from mammalian intestinal mucosa having a sulfation degree and an anticoagulant activity lower than those of the heparin. The present invention includes the use of sulodexide and of the medicinal compositions containing it, in the treatment of COVID-19+ patients suffering thrombosis.

The pharmaceutical compositions having sulodexide can be administered by oral route preferred in carrying out the present invention are capsules, made by soft or hard gelatine, gastroresistant capsules, tablets, controlled release tablets, gastroresistant tablets, granulates and syrups.

The sulodexide dosage, depending on the body weight and the seriousness of the illness, is comprised between 500 L.S.U. (lipasaemic units) and 2000 L.S.U. a day.

The present invention also relates to the use of an inflammation inhibitory amount of an agent selected from the phospholipid sphingosine-1-phosphate (S1P) derivatives of S1P, and mimetics of the S1P or of the derivatives, and pharmaceutically acceptable salts thereof and derivatives thereof, in the treatment of COVID-19 infections. Derivatives of S1P include, without limitation, those disclosed in U.S. Pat. No. 5,260,288: N,N-dimethylsphingosine-1-phosphate, N,N,N,-trimethylsphingosine-1-phosphate, N-acylsphingosine-1-phosphate, sphingosine-1,3-diphosphate, sphingosine-3-phosphate, sphingosine-1-thiophosphate, N,N-dimethyl sphingosine-1-thiophosphate, N-acyl sphingosine-1-thiophosphate and N,N,N-trimethyl sphingosine thiophosphate.

In one embodiment, the present invention provides for a composition for treating a COVID-19 infection, the composition including one or more inhibitors of granzyme B, TNF, HSP 70, interleukin-18 (IL-18 or IL 18), interleukin-10 (IL-10 or IL 10) or elastase 2, and a pharmaceutically acceptable carrier. In aspects of the present invention, the composition may also be used for treating, preventing or minimizing complications associated with COVID-19.

This invention provides a method for treating COVID-19 infections in a subject by administering to the subject one or more inhibitors of one or more of the biomarkers of the present invention, in combination with a second agent. One or more inhibitors of granzyme B, TNF, HSP 70, interleukin-18 (IL-18 or IL 18), interleukin-10 (IL-10 or IL 10) or elastase 2. The inhibitors may be formulated for oral administration, for administration by injection, for topical administration, inhalation.

An inhibitor of granzyme B is a substance that will inhibit or slow down the cleavage of extracellular proteins by granzyme B (U.S. Pat. No. 9,060,960). For example, a compound or composition that prevents granzyme B from cleaving fibronectin, elastin and/or fibrillin is a granzyme B inhibitor. In many cases, inhibitors are referred to as antagonists. Examples of inhibitors of granzyme B described in international patent application published under WO 03/065987 and United States patent application published under US 2003/0148511; Willoughby C A. et al. Bioorg. Med. Chem. Lett. 12:2197-2200 (2002); Hill G E. et al. J. Thorac. Cardiovasc. Surg. 110:1658-1662 (1995); Sun J. et al. J. Biol. Chem. 271:27802-27809 (1996); Sun J. et al. J. Biol. Chem. 272:15434-15441 (1997); Bird et al. Mol. Cell. Biol. 18, 6387-6398 (1998); Kam et al. Biochim. Biophys. Acta 1477:307:23 (2000); and Bio-x-IEPDP-(OPh)2 as described in Mahrus S. and Craik C S. Chemistry & Biology 12:567-577 (2005). Antisense oligonucleotides directed against granzyme B have been designed and manufactured by Biognostik (Euromedex, Mundolshei, France) and are described in Hernandez-Pigeon, et al., J. Biol. Chem. 281: 13525-13532 (2006) and Bruno, et al., Blood, 96: 1914-1920 (2000). Further examples of granzyme B inhibitors are: Z-AAD-CMK (IUPAC name: 5-chloro-4-oxo-2-[2-[2-(phenylmethoxycarbonylamino)propanoylamino]propanoylamino]pentanoic acid) MF: C19H24ClN3O7 CID: 16760474; Ac-IEPD-CHO; Granzyme B Inhibitor IV or Caspase-8 inhibitor III (IUPAC: (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid) MF: C22H34N4O9 OD: 16760476; and Ac-IETD-CHO; Caspase-8 Inhibitor 1 or Granzyme B Inhibitor II (IUPAC: (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid) MF: C21H34N4010 OD: 16760475. Methods of identifying a granzyme B inhibitor are described in U.S. Pat. No. 9,060,960.

Granzyme B inhibitors are described in the following documents: U.S. Pat. No. 7,326,692, US 2006/0019945, US 2007/0104699 A1, U.S. Pat. No. 9,060,960 B2, U.S. Pat. Nos. 10,537,652, 10,246,487, 9,458,193, 9,458,192, 9,458,138, 9,969,772, 9,969,770, 9,849,112, US 2019/0038602, US 2020/0016125.

Granzyme B inhibitors include also protease inhibitors, including soybean-based protease inhibitors. Non-limiting examples of protease inhibitors include Kunitz-type protease inhibitor, Bowman-Birk type protease inhibitors.

TNF inhibitors are well known in the art, and include soluble cytokine receptor that blocks TNF-α activity, (ii) a monoclonal antibody that blocks TNF-α activity, or (iii) a tetracycline or a chemically modified tetracycline that blocks TNF-α activity. Examples of TNF inhibitors include Humira, AP301, OPRX-106.

Example of Hsp70 inhibitors include MAL3-101, MKT-077, VER-155008, Apoptozole, Pifithrin-μ, NSC 630668-R/1, the Fatty acid synthase inhibitor, FASNALL, the DAPK3 inhibitor HS38 and HS72, an allosteric inhibitor selective for Hsp70i (Haystead, T. A. J., Methods Mol Biol. 2018; 1709: 75-8). Other inhibitors include: Elesclomol (STA-4783) is a small molecule, Minnelide, a water-soluble pro-drug of triptolide (active compound from a Chinese herb), PAT-SM6 is an anti-GRP78 monoclonal antibody. Spanidin is a small molecule that binds HSP70. Inhibitors of Hsp 70 are described in US 2006/0074063, U.S. Pat. No. 8,754,094 B2, U.S. Pat. No. 8,486,697, WO 2012/018862, U.S. Pat. Nos. 9,878,987, 10,221,171, US 2016/0368975, WO 2019/173394, US 2019/0282600.

Inhibitors of IL-18 include IL-18 binding protein, an antibody against IL-18, an antibody against an IL-18 receptor subunits, an inhibitor of the IL-18 signaling pathway, an antagonist of IL-18 which competes with IL-18 and blocks the IL-18 receptor, an inhibitor of caspase-1 (ICE), an IL-18 isoform, an IL-18 mutein, an IL-18 fused protein, an IL-18 functional derivative, an IL-18 active fraction, and an IL-18 circularly permutated derivative thereof inhibiting the biological activity of IL-18. Examples include CERC-007 fully humanized anti-IL-18 monoclonal antibody, GSK 1070806, a humanised IgG1/kappa, anti-interleukin 18 monoclonal antibody, Tadekinig alfa is a recombinant human interleukin-18 binding protein, VT-384 is a protein that has been derived from Yatapoxvirus. Example of an IL-18 inhibitor are found in U.S. Pat. No. 8,431,130, US 2016/0215048, US 2018/0127494, U.S. Pat. No. 7,655,616, US 2004/0076628.

Inhibitors of elastase 2 (human neutrophil elastase) include BAY85-8501 small molecule inhibitor, CHF6333 small molecule inhibitor, Debio 9701 engineered protein inhibitor, Elafin protein inhibitor, MPH-966 is a small molecule inhibitor, POL6014 is peptide inhibitor. Inhibitors of elastase 2 include also protease inhibitors, including soybean-based protease inhibitors. Non-limiting examples of protease inhibitors include Kunitz-type protease inhibitor, Bowman-Birk type protease inhibitors.

Inhibitors of IL10 include Rituximab is a chimeric mouse antihuman CD20 antibody.

In another embodiment, a method of treating COVID-19 in a patient comprises administering to the patient an effective amount of tryptophan, arginine, sarcosine and/or LysoPCs or any combination thereof. In embodiments, a subject diagnosed with COVID-19 may be treated with an effective amount of tryptophan, an effective amount of arginine, an effective amount of sarcosine and/or an effective amount of LysoPCs, or any possible combination thereof.

The one or more biomarker of the present invention may be used to prognosticate: Patients die from withdrawal of care after there has been no improvement in lung function (2-3 weeks). These biomarkers help determine who will have a bad outcome earlier and aid end of life decision making, or determine whom will do well and guide persistent management.

The one or more biomarker of the present invention may be used for disease stratification: biomarkers will aid, which is critically important for clinical trials (i.e., who should be enrolled—if their likelihood of death is high regardless, enrolling them will skew the data and therapies may not appear to work—but, they were going to die regardless of treatment).

In order to aid in the understanding and preparation of the within invention, the following illustrative, non-limiting, examples are provided.

### EXAMPLES

### General Methods for all Examples

Study participants and clinical data: The following studies were approved by the Western University, Human Research Ethics Board. We enrolled consecutive patients who were admitted to our level-3 academic ICU at London Health Sciences Centre (London, Ontario) and were suspected of having COVID-19 based on standard hospital screening procedures (13). We collected daily blood samples starting on admission and up to 3 days in COVID-19− patients, or 7 days in COVID-19+ patients (1 additional blood draw on day 10). COVID-19 status was confirmed as part of standard hospital testing by detection of two SARS-CoV-2 viral genes using polymerase chain reaction (14). Patient baseline characteristics were recorded on admission and included age, sex, comorbidities, medications, hematologic labs, creatinine, arterial partial pressure to inspired oxygen (P/F) ratio, and chest x-ray findings. We calculated Multiple Organ Dysfunction Score (MODS) (15) and Sequential Organ Failure Assessment (SOFA) Score (19) for both COVID-19+ and COVID-19− patient groups to enable objective comparison of their illness severity. We categorized both patient groups as having confirmed or suspected sepsis diagnosis using Sepsis 3.0 criteria (16). We also recorded clinical interventions received during the observation period including use of antibiotics, anti-viral agents, systemic corticosteroids, vasoactive medications, VTE prophylaxis, anti-platelet or anti-coagulation treatment, renal replacement therapy, high flow oxygen therapy, and mechanical ventilation (invasive and non-invasive). Final participant groups were constructed by age- and sex-matching COVID-19+ patients with COVID-19− patients and healthy controls that were previously banked in the Translational Research Centre, London, Ontario (https://translationalresearchcentre.com/) (17, 18).

Blood draws: Standard operating procedures were used to ensure all samples were treated rapidly and equally. Blood was obtained from critically ill ICU patients via indwelling catheters daily in the morning and placed immediately on ice. If a venipuncture was required, research blood draws were coordinated with a clinically indicated blood draw. In keeping with accepted research phlebotomy protocols for adult patients, blood draws did not exceed maximal volumes (19). Once transferred to a negative pressure hood, blood was centrifuged and plasma isolated, aliquoted at 250 μL, and frozen at −80 C. All samples remained frozen until use and freeze/thaw cycles were minimized.

### Example 1

Methods

Analyte Measurements: Levels of 57 inflammatory analytes were elucidated using multiplexed biomarker immunoassay kits according to manufacturers' instructions (MilliporeSigma, 400 Summit Drive, Burlington, Mass., 01803, USA) or enzyme-linked immunosorbent assay (ELISA). For the former, plasma inflammatory analytes were measured using a Bio-Plex™ 200 Suspension Array system (Bio-Rad Laboratories, 1000 Alfred Nobel Drive, Hercules, Calif., 94547, USA), which utilized Luminex® xMAP™ fluorescent bead-based technology (Luminex Corp., 12212 Technology Blvd, Austin, Tex., 78727, USA). Bioanalyte concentrations were calculated from standard curves using 5-parameter logistic regression in Bio-Plex Manager 6.1 software. For the latter, plasma levels of TIMP1 (R&D Systems Duo Set #DY970-05, diluted 1:100 or 1:200), TIMP2 (R&D Systems Duo Set #DY971, diluted 1:100) and TIMP3 (R&D Systems Duo Set #DY973, diluted 1:3 or 1:4) were measured with ELISA.

Analyses: Medians (IQRs) and frequency (%) were used to report ICU patient baseline characteristics for continuous and categorical variables, respectively; continuous variables were compared using Mann-Whitney U tests (or Kruskal-Wallis tests, as appropriate), and categorical variables were compared using Fisher's exact chi-square, with P-values <0.05 considered statistically significant. Given the number of analytes processed, we used 2 complimentary methods, traditional population statistics (M.M.) and machine learning (M.D.). Daily analyte concentrations were also reported as medians (IQRs), and comparisons between groups were examined using Mann-Whitney U tests. Given the number of analytes analyzed and the risk of false positives, a P-value of <0.01 was used as our standard for statistical significance. All analyses were conducted using SPSS version 26 (IBM Corp., Armonk, N.Y., USA).

Receiver operating characteristic (ROC) curves were conducted to determine sensitivity and specificity of individual proteins for predicting outcome (alive or dead). Area-under-the-curve (AUC) was calculated as an aggregate measure of protein performance across all possible classification thresholds. All analyses were conducted using SPSS version 26 (IBM Corp., Armonk, N.Y., USA).

Machine Learning: COVID-19 analyte data of the 57 inflammatory analytes elucidated using multiplexed biomarker immunoassay were visualized with a nonlinear dimensionality reduction on the full data matrix using the t-distributed stochastic nearest neighbor (t-SNE) embedding algorithm (20). t-SNE assumes that the “optimal” representation of the data lies on a manifold with complex geometry, but low dimension, embedded in the full dimensional space of the raw data. For feature selection, we pooled analyte data across 1-3 ICU days for each of the COVID-19+ and COVID-19− cohorts and normalized observations within analyte. A random forest classifier was trained on the variables to predict COVID-19 status. A random forest is a set of decision trees and, consequently, we were able to interrogate this collection of trees to identify the features that have the highest predictive value (viz., those features that frequently appear near the top of the decision tree). We limited the decision trees to a maximum depth of five levels and constrained the forest to 50 trees to avoid overfitting the small dataset. We further explored the ability to perform automated classification of COVID-19+ versus COVID-19− patients from their analyte spectra, conservatively employing only a single decision tree and limiting the maximum tree depth to three levels. We trained and tested the classifier using a five-fold cross-validation approach.

Results

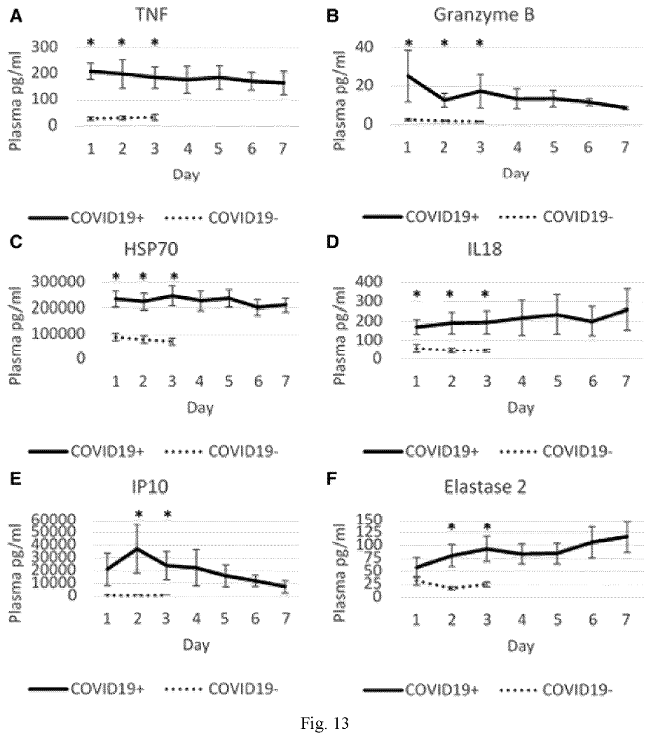

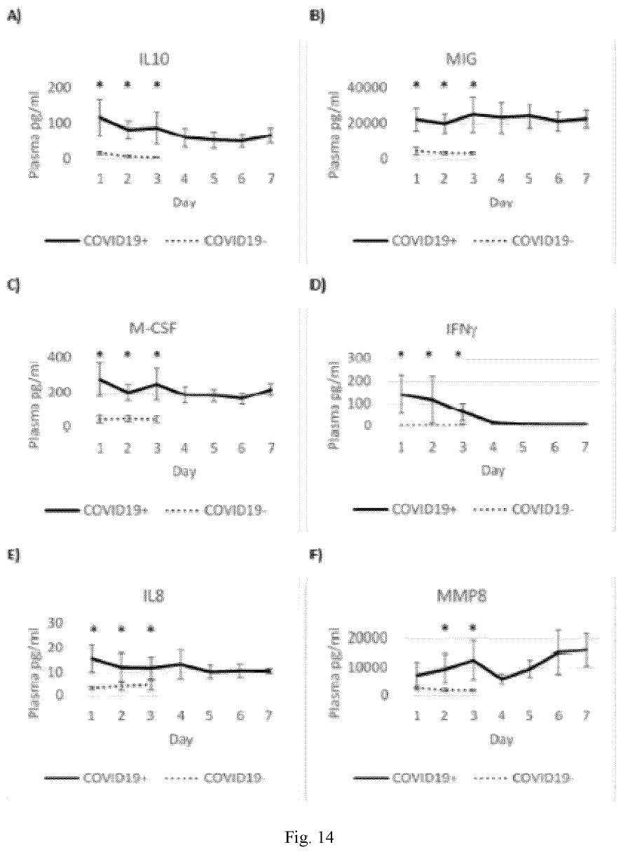

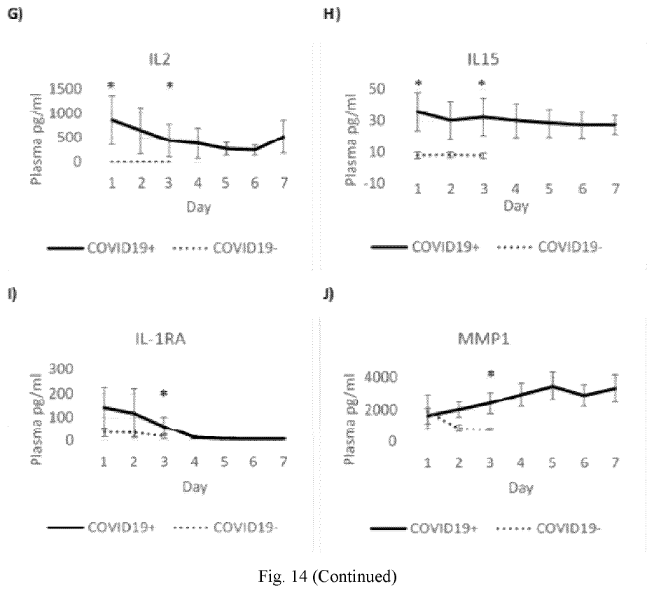

We measured 57 inflammatory analytes in plasma using either fluorescent bead-based multiplex technology or ELISAs. Table 2 shows that 20 inflammatory analytes were significantly different between COVID-19+ ICU patients and healthy controls (the remaining 37 nonsignificant analytes are not shown). All significantly different analytes were elevated in COVID-19+ ICU patients relative to healthy controls except MMP2 that was decreased. COVID-19+ and COVID-19− cohorts were then plotted in two dimensions following dimensionality reduction by stochastic neighbor embedding (FIG. 12). The dimensionality reduction shows that the daily analyte measurements (ICU days 1-3) between the two cohorts are distinct and easily separable. To determine which analytes were most informative for COVID-19 status classification, we performed feature selection with a random forest classifier. The top six features were identified for the binary outcome of COVID-19+ versus COVID-19− in the following order: tumor necrosis factor (TNF), granzyme B, heat shock protein 70 (HSP70), interleukin-18 (IL-18), interferon-gamma-inducible protein 10 (IP-10), and elastase 2 (Table 21). We then trained and tested a simple decision-tree classifier that yielded a classifier accuracy, or the ability of the analytes to predict COVID-19 status, of 98% (p<0.001, five-fold cross-validation). Table 3 lists 17 inflammatory analytes that were significantly different between COVID-19+ and COVID-19− patients on any or all of ICU days 1-3 (the remaining 40 nonsignificant analytes for ICU days 1-3 are not shown). All significant analytes were elevated in COVID-19+ ICU patients relative to COVID-19− ICU patients. While many analytes were significantly different between COVID-19+ and COVID-19− patients over time, the top six analytes determined by feature classification over ICU days 1-3 are listed first, and were TNF, granzyme B, HSP70, and IL-18. IP-10 and elastase 2 were also significantly different between COVID-19+ and COVID-19− patients but starting on ICU day 2. A time course for these six markers is shown in FIG. 13A to 13F over ICU days 1-3 for 5 COVID-19− patients and over ICU days 1-7 for COVID-19+ patients. The mean values for these six analytes remained elevated in COVID-19+ patients across all seven ICU days. The remainder of the analytes measured are shown in FIGS. 14A to 14J, with some analytes increasing (e.g., MMP1, FIG. 14J) and some decreasing (e.g., IFNγ (FIG. 14D) and IL-1RA (FIG. 14I)) over seven ICU days. The feature matrix for day 1 COVID-19+ ICU patients was classified for mortality using a Random Forest classifier (thousand trees) and three-fold cross-validation. As HSP70 was the leading analyte associated with COVID-19+ death, a ROC curve was then conducted to determine the sensitivity and specificity of HSP70 for predicting mortality. As shown in Table 4, AUC for HSP70 was 1.00, indicating perfect sensitivity and specificity for our 10 COVID-19+ ICU patients. Using Youden's Index, the HSP70 cutoff value for predicting mortality was >264,380 pg/mL. Of note, with the addition of the 10 COVID-19− cases to the analysis, the AUC and the cutoff for HSP70 remained the same.

The AUC for IL-1RA, IL10 and MIG was also 1. As such, HSP10, IL-1RA, IL10 and MIG provide perfect sensitivity and specificity for predicting death on ICU day 1 of critically ill COVID-19 patients. These 4 analytes may be taken alone or in combination. Note that other analytes included in Table 4 predicted mortality with high accuracy, such as M-CSF (98%), IL6 (97%) and so forth. As such, in embodiments, a combination of analytes included in Table 4 may be used to determine mortality.

In addition, as seen in Example 2 below, 6 analytes, CLM-1, IL12RB1, CD83, FAM3B, IGF1R and OPTC predicted mortality with high degree of certainty. In other embodiments, any one of the 10 analytes, HSP10, IL-1RA, IL10, MIG, CLM-1, IL12RB1, CD83, FAM3B, IGF1R and OPTC, alone or combination, can be used to predict mortality of COVID-19 patients. In another embodiment, the analytes in Table 4 can be used to predict mortality of COVID-19 patients.

### Example 2

Methods

Proximity Extension Assay (PEA): A total of 1,161 plasma proteins were measured using an immunoassay based on PEA technology (Olink) (21, 22). A 0.25 mL aliquot of citrate plasma obtained from each subject was transported frozen on dry ice to the Clinical Research Laboratory and Biobank (Hamilton, ON). The data generated were expressed as relative quantification on the log 2 scale of normalized protein expression (NPX) values. Individual samples were screened based on quality controls for immunoassay and detection, as well as degree of hemolysis. NPX values were rank-based normal transformed for further analyses. Following proteomic quality control, all 30 participants were deemed suitable for analysis.

Machine Learning: COVID-19 analyte data of 1,161 plasma proteins measured using an immunoassay based on PEA technology, was visualized with a nonlinear dimensionality reduction on the full data matrix using the t-distributed stochastic nearest neighbor embedding (t-SNE) algorithm (23). t-SNE assumes that the ‘optimal’ representation of the data lies on a manifold with complex geometry, but low dimension, embedded in the full dimensional space of the raw data (20). For feature selection, the raw data for each subject were ingested and normalized within each feature, across subjects. More specifically, the data for each marker was scaled to have unit norm. A random forest classifier was trained on the variables to predict COVID status. A random forest is a set of decision trees and, consequently, we were able to interrogate this collection of trees to identify the features that have the highest predictive value (viz., those features that frequently appear near the top of the decision tree). The feature matrix for day one COVID-19 positive ICU patients was classified for patient outcome using a three-fold cross validation with a Random Forest of hundred trees and max depth of 6 to reduce overfitting.

Results

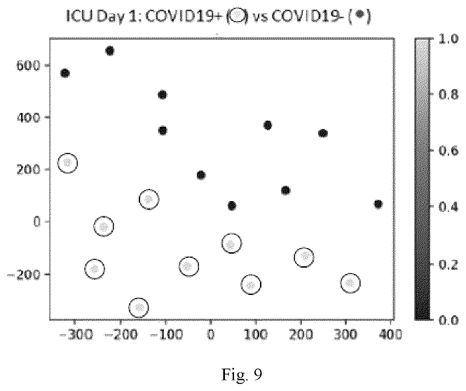

1,161 plasma proteins were measured using PEA immunoassays. FIG. 8 shows a Tsne plot illustrating that the COVID-19+ ICU patient proteome (a circle was used to better visualize the COVID-19+ patients) was distinct and easily separable from age- and sex-matched healthy control subjects. Feature classification identified the top 20 proteins underlying these differences between cohorts and are shown in Table 17 with their associated importance. Classification accuracy was 100%. The biological functions of these leading 20 proteins are described in Table 5.

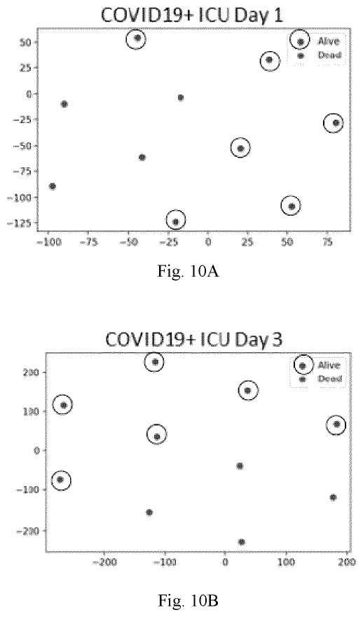

FIG. 9 shows a tSNE plot illustrating that the COVID-19+ ICU patient proteome was distinct and easily separable from age- and sex-matched COVID-19− ICU patients. Feature classification identified the top 20 proteins underlying these differences between cohorts and are shown in Table 18 with their associated importance. Classification accuracy was 100%. The biological functions of these proteins are described in Table 6.

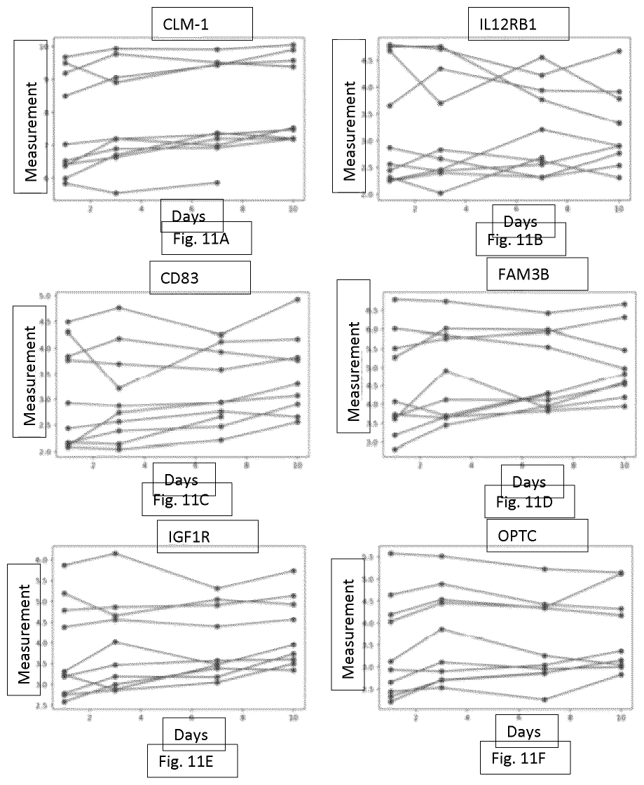

We then determined the ability of the plasma proteome (all 1,161 plasma proteins) to predict mortality in COVID-19+ patients on either ICU days 1 or 3. FIG. 10A shows a tSNE plot demonstrating that the proteome between COVID-19+ patients on ICU day 1 that either survived or died are distinct and easily separable. The top 21 proteins underlying these outcome differences are shown in Table 19, and their biological functions are described in Table 7. FIG. 10B shows a tSNE plot demonstrating that the proteome between COVID-19+ patients on ICU day 3 that either survived or died are distinct and easily separable. The top 21 proteins underlying these outcome differences are shown in Table 20, and their biological functions are described in Table 8. The classification accuracy to predict outcome with the entire 1,161 proteins in COVID-19+ patients on ICU days 1 and 3 was 92% and 83%, respectively.

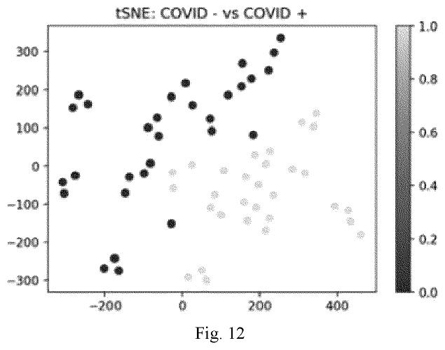

To optimize outcome prediction in COVID-19+ patients, we then narrowed the number of proteins from 1,161 using ROC analyses. The top 6 proteins for predicting ICU survival/death using only a day 1 plasma sample are shown in FIGS. 11A to 11F; also shown is their associated time course over ten ICU days. There were no deaths during the 10 ICU days for either cohort; however, one COVID-19+ ICU survivor was discharged on day 7 with no further plasma measurements. With all 6 proteins, the COVID-19+ patients that died had elevated levels relative to those COVID-19+ patients that survived to ICU discharge. All 6 proteins provided 100% classification accuracy with the following cutoffs: CLM-1 7.8, IL12RB1 3.3, CD83 3.3, FAM3B 4.7, IGF1R 3.8, and OPTC 3.6.

In this study, we measured 1,161 proteins in plasma obtained from ICU patients, both COVID-19+ and COVID-19−, as well as age- and sex-matched healthy controls. Given the number of analytes measured, we analyzed the data with state-of-the-art machine learning. Our data indicate the presence of a unique COVID-19 proteome with 6 proteins predicting ICU mortality with 100% accuracy.

CMRF-35-like molecule 1 (CLM-1), a type-1 transmembrane glycoprotein with an extracellular IgG domain, accurately predicted COVID-19 ICU outcome. CLM-1 is expressed predominantly in myeloid cells where it can impair IL-6 production in bone marrow derived mast cells and promotes phagocytosis of dead cells by binding phosphatidylserine, which serves as a common apoptotic cell surface recognition cue. The removal of apoptotic cells by CLM-1 expressing macrophages may prevent the generation of secondary necrosis and the release of potentially toxic or immunogenic components from necrotic cells, reducing the likelihood of an inflammatory reaction. IL12RB1, one of two subunits within the IL-12 receptor, is expressed on natural killer (NK) cells and T-cells cells. Essential for resistance to intracellular pathogens, IL12RB mediates the proinflammatory response to IL-12 that is released by antigen presenting cells (24). Individual variability in IL12RB1 function is introduced at the epigenetic, genomic polymorphism, and mRNA splicing levels, thereby inferring disease susceptibility and variable outcomes (25). CD83, a member of the immunoglobulin superfamily, is expressed on a variety of activated immune cells (26). Inferring selective immunosuppression when membrane bound on antigen presenting cells, soluble CD83 infers powerful immunosuppressive properties by inhibiting proliferation and function of T-cells. Viral infection leads to the degradation of dendritic cell CD83, a mechanism described as a viral immune escape mechanism (27). FAM3B, expressed at high levels in the islets of Langerhans of the endocrine pancreas, is a secreted cytokine that induces apoptosis (28). Increased CD38 is associated with pancreatic β cell dysfunction, hyperglycemia and insulin resistance, suggesting a role in the regulation of glucose and lipid metabolism (29). IGF1R, a transmembrane tyrosine kinase receptor that is activated insulin-like growth factor 1, is expressed on lymphocytes and macrophages and can mediate lung injury in response to infectious pathogen or chemical insult. In particular, phosphorylation of the IGF1R receptor exaggerates inflammation and its over-expression aggravates cytokine levels during influenza infection (30). Conversely, IGF1R deficiency attenuates acute inflammatory response in a lung injury mouse model (31). OPTC, also called opticin, is highly expressed in the eye nonpigmented ciliary epithelium that secretes it into the vitreous cavity where it associates with vitreous collagen and adjacent basement membranes (32). As a small leucine-rich protein, OPTC binds collagen fibrils and regulates extracellular matrix adhesiveness to suppress capillary morphogenesis and inhibit endothelial invasion (33). OPTC is also expressed in lymphocytes (34), but its role in infection and inflammation is unknown.

### Example 3

Quantitative assays are used to determine the absolute concentration of the one or more markers listed in Tables 4, 7 and 8, including each of HSP70, IL-IRA, IL 10, MIG, CLM-1, IL12RB1, CD83, FAM3B, IGF1R and OPTC in COVID-19 patients admitted to ICU and in healthy controls in absolute weight or absolute moles per volume. The absolute concentrations in the healthy controls are used to determine absolute concentration threshold that predicts disease severity and mortality. Levels of the one or more markers above the threshold level for the one or more markers concentration in absolute weight or absolute moles per volume indicate that the patient is at risk disease severity including at risk of death.

### Example 4

Methods

Enzyme-Linked Immunosorbent Assay (ELISA): All plasma analytes were measured with immunoassays in duplicate as per the manufacturer's recommendation. Analytes measured include ADAMTS13 (Abcam #ab234559, diluted 1:200), Protein C (Assaypro #EP1311-7, diluted 1:8), von Willebrand factor (vWF; Thermo Fisher #EHVWF, diluted 1:8000), soluble platelet selectin (sP-selectin; Abcam #ab100631, diluted 1:50 or 1:20), heparan sulfate (TSZ ELISA #HU8718, diluted 1:5), chondroitin sulfate (TSZ ELISA #HU8720, diluted 1:2), hyaluronic acid (R&D Systems #DHYALO, diluted 1:20), and syndecan-1 (Abcam, #ab46506, diluted 1:2).

Isolation and culture of human pulmonary microvascular endothelial cells (hPMVEC): hPMVEC were isolated from resected human lung as previously described (35, 36). Briefly, human peripheral lung tissue was finely minced, and digested in 0.3% type II collagenase at 37° C. The digested suspension was filtered, centrifuged, and washed in PBS. Endothelial cells were then isolated using magnetic Dynabeads coated with anti-human CD31 antibody. Isolated cells were resuspended in EGM-2 (Lonza #CC-3162) with 10% fetal bovine serum and placed at 37° C. in 5% CO2 until 50% confluent, then harvested and re-purified using anti-CD31-coated magnetic microbeads as above. PMVEC were propagated in EGM-2+10% FBS and 20 mM HEPES on fibronectin-coated flasks and passages 4-9 used.

Hyaluronidase treatment of hPMVEC: PMVEC (2.5×10⁴/well) were plated on fibronectin-coated 4-well plates in EGM-2+10% FBS and 20 mM HEPES. After 2 days, media was changed to HBSS (+100 mM HEPES, no bicarb)+0.01% BSA and hPMVEC were treated for 1 h with hyaluronidase (0.5 mg/mL; Sigma #H3506). Following treatment, hPMVEC were loaded with a nitric oxide-sensitive fluorochrome (2 μM DAF-FM DA (Thermo Fisher #D23844) for 1h before lysing with 0.5% SDS in PBS. After centrifugation for 10 min at 1×10⁴RCF, the fluorescence of triplicate aliquots of supernatant were measured using a Victor3 multilabel fluorescence microplate reader (Wallac Oy; Perkin Elmer, Inc.) at 485/520 for DAF-FM. The nitric oxide donor DETA NONOate (20 μM; Cayman #82120) was used as the positive control.

Population Statistics: Medians (IQRs) and frequency (%) were used to report continuous and categorical variables, respectively. Continuous variables were compared using either the Mann-Whitney U test or the Kruskal-Wallis test, as appropriate, and categorical variables were compared using Fisher's exact chi-square. P-values <0.05 considered statistically significant. All population statistics were conducted using SPSS version 26 (IBM Corp., Armonk, N.Y., USA). For data comparison that were non-significant, G*Power version 3.1.9.4 was used to determine the number of patients per cohort required to potentially reach statistical significance based on measured values and 80% power (37).

Machine Learning: Nonlinear dimensionality reductions on the full datasets to only two dimensions was completed using the t-distributed stochastic nearest neighbor (t-SNE) algorithm (38). For classification, we pooled analyte data across days 1-3 for each of the COVID-19+ and COVID-19− cohorts and normalized observations within-analyte. A random forest classifier was trained on the variables to predict COVID status. In addition, another random forest classifier was trained on pooled analyte data for COVID-19+ patients for days 1-3 to predict patient mortality. A random forest is a set of decision trees that we can interrogate to identify the features with the highest predictive value. We limited the decision trees to a maximum depth of 6 levels and constrained the forest to 10 trees in order to avoid overfitting the small dataset. We trained and tested the classifier using a 5-fold cross-validation approach.

Results

We investigated 10 patients with a positive diagnosis of COVID-19 (median years of age=61.0, IQR 54.8, 67.0), 10 age- and sex-matched patients with a negative diagnosis of COVID-19 (median years of age=58.0, IQR 52.5, 63.0), and 10 age- and sex-matched healthy controls (median years of age=57.5, IQR 52.8, 62.8; P=0.686). Baseline demographic characteristics, comorbidities, labs, and chest x-ray findings are reported in Table 1. COVID-19+ patients relative to COVID-19− patients were more likely to have bilateral pneumonia (P=0.001). Pathogens were confirmed in only 2 of the COVID-19− patients (P=0.001). All other reported baseline measures were non-significant between patients.

We measured 3 thrombosis factors and 5 endothelial cell injury markers in plasma using ELISAs. Table 9 shows that 3 markers (vWF, chondroitin sulfate, and syndecan-1) were significantly elevated in COVID-19+ ICU patients relative to healthy controls. Table 10 lists the plasma measurements for 8 markers between COVID-19+ and COVID-19− patients on ICU days 1-3. Significant elevations were only observed in endothelial injury biomarkers, including sP-selectin (ICU day 3), heparan sulfate (ICU day 2), hyaluronic acid (ICU day 3) and syndecan-1 (ICU days 1-3).

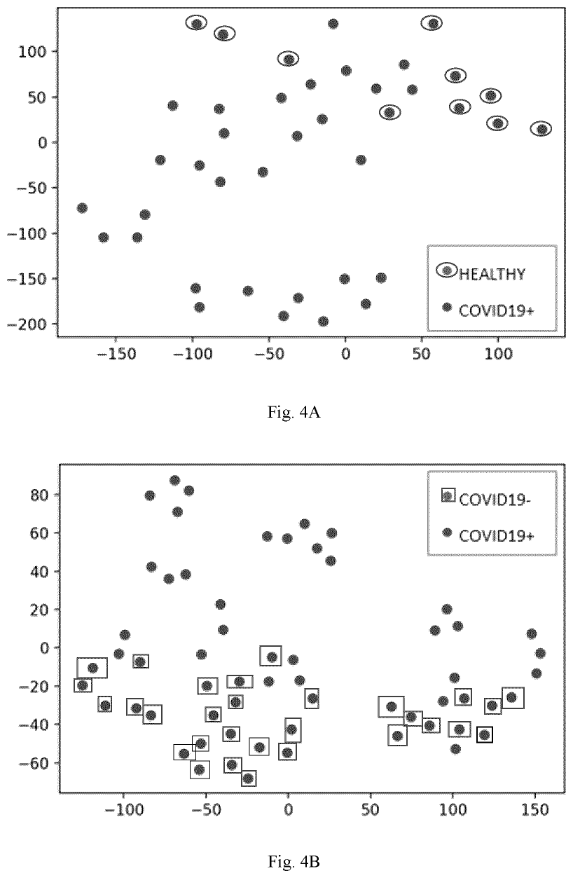

We then reduced the data to two-dimensions using t-SNE to visualize differences between healthy controls and COVID-19+ patients (ICU days 1-3; FIG. 4A), as well as COVID-19− and COVID-19+ patients (ICU days 1-3; FIG. 4B). In both cases, the COVID-19+ patients were easily distinguishable from either healthy controls or COVID-19− patients. We then trained and tested a random forest classifier that yielded a classifier accuracy, or the ability of the markers to predict COVID-19 status, of 85% (5-fold cross-validation). To determine which of the 8 markers were most informative for COVID-19 status classification, we undertook feature selection with the random forest classifier. For ICU days 1-3, the top features in rank order were identified for the binary outcome of COVID-19+ versus COVID-19− as: syndecan-1>hyaluronic acid>chondroitin sulfate>ADAMTS13>heparan sulfate>Protein C>sP-selectin>vWF. However, for ICU day 3 only, the top features in rank order were hyaluronic acid>sP-selectin>syndecan-1>>ADAMTS13>chondroitin sulfate=heparan sulfate>vWF>Protein C.

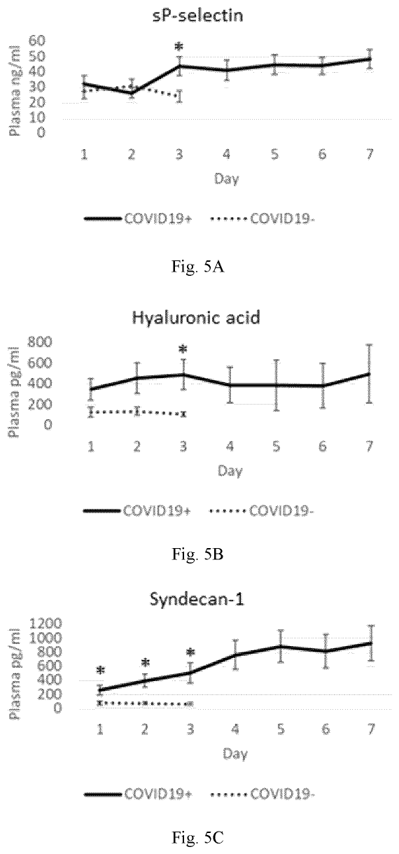

Given the significant elevation in plasma hyaluronic acid, sP-selectin and syndecan-1 on ICU day 3, we continued daily plasma measurements until ICU day 7 (FIG. 5). For all three endothelial injury biomarkers, the plasma levels remained elevated suggesting ongoing glycocalyx degradation.

To determine a relationship between the thrombotic state and outcome, we trained and tested a random forest classifier to determine the ability of the 8 markers on ICU days 1-3 to predict mortality in COVID-19+ patients. The thrombosis profile yielded a classifier accuracy, or the ability of the markers to predict mortality, of 86% (5-fold cross-validation).

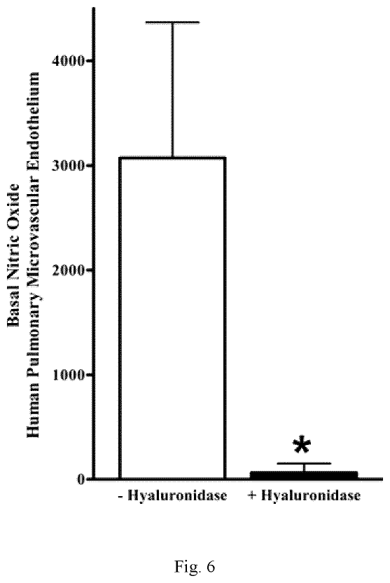

Given the reliance of the classification accuracy on hyaluronic acid degradation, and the reports of injury to the pulmonary endothelium with COVID-19, we specifically removed hyaluronic acid from human hPMVEC with hyaluronidase treatment (FIG. 6). Hyaluronidase treatment decreased basal intracellular nitric oxide production by 98% to 64±87.5 RFUs, compared to untreated human PMVEC (P=0.008, n=5 separate experiments). The positive control DETA NONOate (20 μM) increased nitric oxide production by 16% compared to untreated controls (data not shown).

Table 11 shows a comparison of healthy controls, COVID-19− and COVID-19+ patients on ICU day 3. HA and syndecan-1 were significantly elevated in COVID-19+ patients on ICU day 3.

In this study, we measured 3 thrombotic factors and 5 endothelial cell injury markers in plasma obtained from ICU patients, both COVID-19+ and COVID-19−, as well as age- and sex-matched healthy controls. Our data indicate increased vWF in COVID-19+ patients relative to healthy controls elevated sP-selectin, hyaluronic acid and syndecan-1.

Our COVID-19+ ICU patients were similar to those reported in earlier cohorts from multiple countries with respect to age, comorbidities and clinical presentation (39, 40-42). In contrast to COVID-19− ICU patients, our COVID-19+ ICU patients had a higher incidence of bilateral pneumonia (43). The COVID-19+ patients in our study appeared to have lower illness severity scores than the COVID-19− patients, yet mortality was high at 40%. In contrast, all COVID ICU patients survived. Although these differences were not statistically significant, the findings suggest that acute respiratory distress syndrome in COVID-19+ patients has worse outcomes, perhaps due to the persistently high levels of plasma serine proteases (43).

Our data suggest that COVID-19 results in endothelial injury. Specifically, sP-selectin, hyaluronic acid, and syndecan-1 were all significantly elevated by ICU day 3 in plasma of COVID-19+ patients relative to COVID-19− patients and remained persistently elevated in plasma up to ICU day 7.

Syndecan-1 is a proteoglycan containing both heparan- and chondroitin-sulfate chains that mediates cellular responses to signaling molecules as well as cell-cell and cell-matrix interactions (44). During inflammation, syndecan-1 functions to inhibit neutrophil adhesion and migration. Shedding of syndecan-1 from the cell surface is initiated by heparanase-dependent removal of the heparan-sulfate side chains (45), thereby instigating subsequent cleavage of the core syndecan-1 protein by enzymes such as matrix metalloproteinases. Importantly, moderate syndecan-1 shedding is thought to aid in resolving inflammation; however, excessive shedding is likely pathogenic as complete loss of syndecan-1 allows for increased leukocyte adhesion and recruitment across the endothelial monolayer, as well as enhanced platelet aggregation and adhesion. Different sheddases are able to cleave syndecans on the extracellular side, releasing a soluble syndecan consisting of the extracellular domain and the attached GAG chains (syndecan-degradation products).

Our study has identified a unique pro-thrombotic state in critically ill COVID-19 patients that may be amenable to therapeutic targeting.

Our study, taken in the context of the current literature, suggests that ‘not all coagulopathy is created equal’. While some patients may develop an extreme pro-thrombotic state secondary to the development of either anticardiolipin antibodies (46) or activated plasminogen (47), others may be pro-thrombotic on the basis of alveolar-capillary membrane denudation and exposure of tissue factor (48). Anticoagulants are one treatment strategy; however, low molecular weight heparin did not confer an overall survival advantage in COVID-19 patients (49). The beneficial effects of specific therapeutic strategies may be diluted by patient and disease heterogeneity, suggesting that a personalized treatment approach is required.

### Example 5

Preamble

COVID-19 presents clinical symptoms that share features of Kawasaki's Disease (KD) and are attributable in part to an acute vasculopathy. A ‘cytokine storm’ has been suggested to underlie the syndrome, with tissue injury secondary to the host innate response (50). The inflammatory and endothelial injury mediators have not yet been described, but knowledge of these analytes is critically important for earlier syndrome recognition and for potential interventions.

Results

A 15-year-old female presented to hospital to a tertiary care emergency department with a history of malaise, dry cough, strawberry tongue, rash and jaundice. COVID-19 was confirmed by detection of two SARS-CoV-2 viral genes using polymerase chain reaction. Her complete blood count, electrolytes, coagulation profile and blood gas were normal. C-reactive protein and ferritin were mildly elevated at 25.7 mg/L and 302 μg/L, respectively. She had a mild hepatitis with alanine aminotransferase 142 U/L, aspartate aminotransferase 87 U/L, alkaline phosphate 405 U/L, total bilirubin 92.6 μmon. She was admitted to hospital with a presumptive diagnosis of atypical KD and treated with intravenous immunoglobulin (IVIg) and Aspirin. Her inpatient electrocardiogram and echocardiogram were normal.

Blood was drawn for inflammation/endothelial injury profiling after the patient's COVID-19 status was confirmed, but IVIg had already been administered approximately 48 hours earlier. Thus, analyte measurements must be evaluated in the context of this immune modulator (see below). Nonetheless, 59 inflammation- and endothelium-related analytes were measured using multiplexed biomarker immunoassay kits or enzyme-linked immunosorbent assay (ELISA). As only one COVID-19 pediatric patient was admitted to our hospital, we compared the measured analyte values from this COVID-19 case patient to analyte reference ranges that we obtained from a cohort of 20 pediatric healthy control subjects [median 15 years of age (IQR 8)].

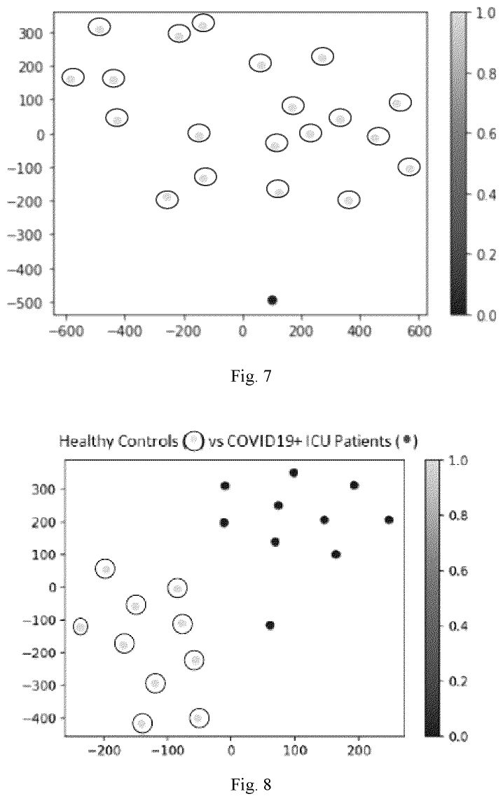

The analyte data from the COVID-19 patient and the 20 healthy control subjects were first visualized with a nonlinear dimensionality reduction on the full data matrix using the t-distributed stochastic nearest neighbour (t-SNE) embedding algorithm (FIG. 7). t-SNE assumes that the ‘optimal’ representation of the data lies on a manifold with complex geometry, but low dimension, embedded in the full dimensional space of the raw data. Based on analyte measurements, the COVID-19 case patient is a clear outlier with respect to her inflammation and endothelial injury profile.

We then generated confidence intervals (CIs) for the expected value of each analyte using the plasma measurements from the 20 healthy pediatric controls. The plasma values for each analyte were not normally distributed, so we computed 99.9% (95%, Bonferonni corrected for comparison across 59 plasma analytes) CIs via the bias corrected and accelerated bootstrap. Plasma analyte values in the COVID-19 case patient that were outside the CIs for healthy control subjects were therefore considered significant (p<0.05, corrected; Table 12. We found significant elevations in 21 inflammation and endothelial analyte markers, while 1 endothelial glycocalyx degradation marker (heparan sulfate) was significantly depressed (Table 12).

After 3 days of observation, and partial resolution of her symptoms, the COVID-19 case patient was discharged home on Aspirin (3 mg/kg/day) with a 2-week follow up echocardiogram. Matrix metalloproteinase 7 (MMP7) was the most elevated analyte in the COVID-19 case patient relative to healthy control subjects. Also called matrilysin, MMP7 is expressed in endothelial cells, monocytes and macrophages and it is capable of degrading multiple extracellular membrane components (proteoglycans, laminin, fibronectin, casein and basement membrane collagen type IV). MMP7 is significantly upregulated in KD and it is implicated in acute vasculopathy (51). Specifically, MMP7 degrades endothelial junctions, which can promote vascular leak/edema and/or leukocyte migration into tissues (52). MMP7 has been identified as a syndecan-1 sheddase in lung mucosa (62).

Interferon-γ-inducible protein 10 (IP-10), an inflammatory cytokine secreted primarily by monocytes and endothelial cells in response to interferon-γ (IFNγ), was also significantly elevated in the COVID-19 case patient. IP-10 has multiple roles including lymphocyte chemoattraction and adhesion to endothelial cells. IP-10 is a promising target for the treatment of infectious diseases as it aids cellular targeting to threatened tissues where it modulates innate and adaptive immune responses. High serum IP-10 is found in KD, and it has been suggested as a KD biomarker (53). Resistin is highly expressed in macrophages, bone marrow and the non-fat fraction of adipose tissue, and it stimulates several pro-inflammatory pathways and cytokines. Microvascular tone, as well as endothelial cell barrier function and nitric oxide production, are all altered by resistin. Similar to our COVID-19 case patient, elevated resistin is found in plasma from KD patients (54, 55).

Interleukin 3 (IL-3), released by activated T-cells, was elevated in our COVID-19 case patient. IL-3 promotes the production of inflammatory monocytes and neutrophils, thereby contributing to the cytokine storm that is implicated in sepsis from multiple etiologies. The microvascular endothelial cell response to inflammation and immunity is also regulated by IL-3 (56) and vasculopathy is suggested to be a primary feature of the novel multi-system inflammatory syndrome. Hyaluronic acid is a major constituent of the microvascular glycocalyx, an extracellular matrix that coats the luminal surface of the endothelium (57). Hyaluronic acid degradation products are significantly elevated in plasma from the COVID-19 case patient, suggesting that the microvascular endothelial cell luminal surface has been pathologically altered. Disruption of the endothelial glycocalyx is associated with vascular lesions in KD (58), as well as decreased endothelial nitric oxide production and increased platelet/endothelium adhesion (57), Endothelial cell injury was supported in the COVID-19 case patient by the parallel elevation of soluble P-selectin, an endothelial glycoprotein that mediates adhesive intercellular interactions (59).

Our measurements showed minimal alterations in 37 inflammation and endothelial analyte markers: epidermal growth factor (EGF), granulocyte-colony stimulating factor (G-CSF), granulocyte-macrophage colony-stimulating factor (GM-CSF), IFNγ, interleukin 1a (IL-1a), IL-1b, IL-2, IL-4, IL-5, IL-6, IL-7, IL-8, IL-10, IL-12(p40), IL-12(p70), IL-13, IL-15, IL-17a, IL17e/IL25, IL-17f, IL-22, macrophage colony-stimulating factor (M-CSF), macrophage inflammatory protein 1 a (MIP-1a), tumor necrosis factor α (TNFα), TNFβ, vascular endothelial growth factor A (VEGFA), regulated upon activation, normal T Cell expressed and presumably secreted (RANTES), MMP2, MMP3, MMP9, MMP12, MMP13, neutrophil gelatinase-associated lipocalin (NGAL), Granzyme B, heat shock protein 70 (HSP-70), chondroitin sulfate and syndecan-1. As some of these measurements may have been depressed by IVIg administration (60), no significant conclusions can be made with regards to their pre-treatment level. It is also plausible that some inflammatory analytes were transiently increased with inflammation onset, with TNF and IL-6 as typical examples (61). TNF-α is a pro-inflammatory cytokine released primarily by monocytes and macrophages that enhances the adaptive immune response. IL-6 is produced by monocytes and macrophages, and induces T-cell activation, B cell proliferation and stimulates the acute phase reaction, all of which lead to augmentation of the immune response. In summary, pediatric COVID-19 patients can present with a novel multi-system inflammatory syndrome with some features similar to KD. The analyte measurements presented in this study, albeit post IVIg treatment, support a systemic inflammatory process that resulted in significant endothelial injury. These data should aid future hypothesis-generating research, as some of the identified analytes might be putative disease biomarkers and/or potential therapeutic targets.

### Example 6

Methods

DI-LC-MS/MS: A targeted quantitative metabolomics approach was used to analyze the samples using a combination of direct injection mass spectrometry with a reverse-phase LC-MS/MS custom assay. This custom assay, in combination with an ABSciex 4000 QTrap (Applied Biosystems/MDS Sciex) mass spectrometer, can be used for the targeted identification and quantification of up to 150 different endogenous metabolites including amino acids, acylcarnitines, biogenic amines & derivatives, uremic toxins, glycerophospholipids, sphingolipids and sugars (63, 64). The method combines the derivatization and extraction of analytes, and the selective spectrometric detection using multiple reaction monitoring (MRM) pairs. Isotope-labeled internal standards and other internal standards are used for metabolite quantification. The custom assay contains a 96 deep-well plate with a filter plate attached with sealing tape, and reagents and solvents used to prepare the plate assay. First 14 wells were used for one blank, three zero samples, seven standards and three quality control samples. For all metabolites except organic acid, samples were thawed on ice and subsequently vortexed and centrifuged at 13,000×g; 10 μL of each sample was then loaded onto the center of the filter on the upper 96-well plate and dried in a stream of nitrogen. Subsequently, phenyl-isothiocyanate was added for derivatization. After incubation, the filter spots were dried again using an evaporator. Extraction of the metabolites was then achieved by adding 300 μL of extraction solvent. The extracts were obtained by centrifugation into the lower 96-deep well plate, followed by a dilution step with MS running solvent.

For organic acid analysis, 150 μL of ice-cold methanol and 10 μL of isotope-labeled internal standard mixture was added to 50 μL of serum sample for overnight protein precipitation. Then it was centrifuged at 13000×g for 20 min. 50 μL of supernatant was loaded into the center of wells of a 96-deep well plate, followed by the addition of 3-nitrophenylhydrazine (NPH) reagent. After incubation for 2 h, BHT stabilizer and water were added before LC-MS injection.

Mass spectrometric analysis was performed on an ABSciex 4000 Qtrap® tandem mass spectrometry instrument (Applied Biosystems/MDS Analytical Technologies, Foster City, Calif.) equipped with an Agilent 1260 series UHPLC system (Agilent Technologies, Palo Alto, Calif.). The samples were delivered to the mass spectrometer by a LC method followed by a direct injection (DI) method. Data analysis was done using Analyst 1.6.2.

¹H NMR: Plasma and serum samples contain a significant concentration of large molecular weight proteins and lipoproteins which affects the identification of the small molecular weight metabolites by NMR spectroscopy. A deproteinization step, involving ultra-filtration as previously described (65), was therefore introduced in the protocol to remove plasma proteins. Prior to filtration, 3 KDa cut-off centrifugal filter units (Amicon Microcon YM-3), were rinsed five times each with 0.5 mL of H₂O and centrifuged (10,000 rpm for 10 minutes) to remove residual glycerol bound to the filter membranes. Aliquots of each plasma sample were then transferred into the centrifuge filter devices and spun (10,000 rpm for 20 minutes) to remove macromolecules (primarily protein and lipoproteins) from the sample. The filtrates were checked visually for any evidence that the membrane was compromised and for these samples the filtration process was repeated with a different filter and the filtrate inspected again. The subsequent filtrates were collected and the volumes were recorded. If the total volume of the sample was under 250 μL an appropriate amount from a 150 mM KH2PO4 buffer (pH 7) was added until the total volume of the sample was 173.5 μL. Any sample that had to have buffer added to bring the solution volume to 173.5 μL, was annotated with the dilution factor and metabolite concentrations were corrected in the subsequent analysis. Subsequently, 46.5 μL of a standard buffer solution (54% D20:46% 1.75 mM KH2PO4 pH 7.0 v/v containing 5.84 mM DSS (2,2-dimethyl-2-silcepentane-5-sulphonate), 5.84 mM 2-chloropyrimidine-5 carboxylate, and 0.1% NaN3 in H2O) was added to the sample. The plasma sample (250 μL) was then transferred 3 mm SampleJet NMR tube for subsequent spectral analysis. All 1H-NMR spectra were collected on a 700 MHz Avance III (Bruker) spectrometer equipped with a 5 mm HCN Z-gradient pulsed-field gradient (PFG) cryoprobe. ¹HNMR spectra were acquired at 25° C. using the first transient of the NOESY pre-saturation pulse sequence (noesyldpr), chosen for its high degree of quantitative accuracy (66). All FD's (free induction decays) were zero-filled to 250 K data points. The singlet produced by the DSS methyl groups was used as an internal standard for chemical shift referencing (set to 0 ppm) and for quantification all ¹H-NMR spectra were processed and analyzed using an in-house version of the MAGMET automated analysis software package using a custom metabolite library. MAGMET allows for qualitative and quantitative analysis of an NMR spectrum by automatically fitting spectral signatures from an internal database to the spectrum. Each spectrum was further inspected by an NMR spectroscopist to minimize compound misidentification and mis-quantification. Typically, all of visible peaks were assigned. Most of the visible peaks are annotated with a compound name. It has been previously shown that this fitting procedure provides absolute concentration accuracy of 90% or better (67).

Population Statistics: Medians (IQRs) and frequency (%) were used to report ICU patient baseline characteristics for continuous and categorical variables, respectively; continuous variables were compared using Mann-Whitney U tests (or Kruskal-Wallis tests, as appropriate), and categorical variables were compared using Fisher's exact chi-square, with P-values <0.05 considered statistically significant. Receiver operating characteristic (ROC) curves were conducted to determine sensitivity and specificity of individual metabolite ratios for predicting a binary outcome. Area-under-the-curve (AUC) was calculated as an aggregate measure of metabolite ratio performance across all possible classification thresholds. All analyses were conducted using SPSS version 26 (IBM Corp., Armonk, N.Y., USA).

Machine Learning: COVID-19 analyte data were visualized with a nonlinear dimensionality reduction on the full data matrix using the t-distributed stochastic nearest neighbor embedding (t-SNE) algorithm (68). t-SNE assumes that the ‘optimal’ representation of the data lies on a manifold with complex geometry, but low dimension, embedded in the full dimensional space of the raw data. For feature selection, the raw data for each subject were ingested within each feature, across subjects. A random forest classifier was trained on the variables to predict COVID-19 status or COVID-19 outcome. A random forest is a set of decision trees and, consequently, we were able to interrogate this collection of trees to identify the features that have the highest predictive value (viz., those features that frequently appear near the top of the decision tree). To reduce overfitting, COVID-19 status was determined using a six-fold cross validation with a random forest of ten trees, whereas, patient outcome was determined using a three-fold cross validation with a random forest of ten trees and max depth of 6 (69).

Results

We investigated 10 COVID-19+ patients (median years of age=61.0, IQR=54.8, 67.0), 10 age- and sex-matched COVID-19− patients (median years of age=58.0, IQR=52.5, 63.0), and 10 age- and sex-matched healthy controls (median years of age=57.5, IQR=52.8, 62.8; P=0.686). Baseline demographic characteristics, comorbidities, laboratory values, and chest x-ray findings are reported in Table 1. The COVID-19− patients had significantly higher unilateral pneumonia, while COVD19+ patients were more likely to have bilateral pneumonia. Sepsis was ‘confirmed’ by infectious pathogen identification in only 20% of COVID-19− patients, whereas sepsis was ‘suspected’ in the remaining 80%. A mortality rate of 40% was determined for COVID-19+ patients.

We measured a total of 183 plasma metabolites using both DI-LC-MS/MS and ¹H NMR. In the event of metabolite repeats measured with both techniques (21 metabolites), the ¹H NMR metabolite repeat measurements were deleted from the combined metabolite database yielding a final number of 162 metabolites analyzed, which are listed in Table 13.

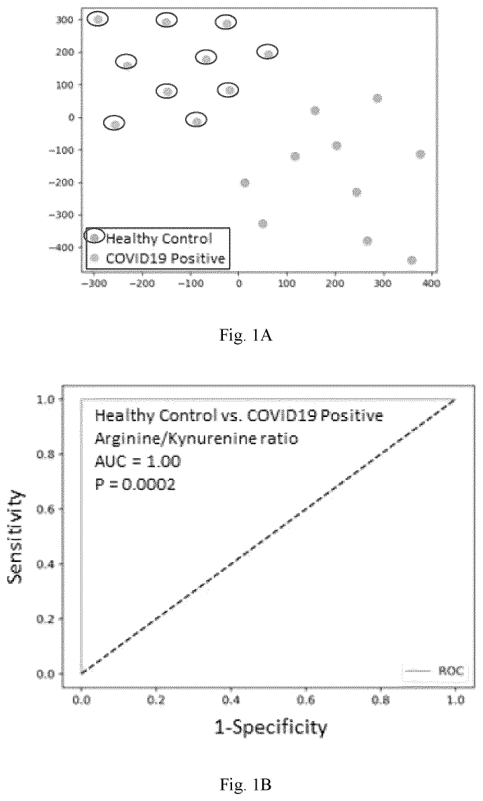

FIG. 1A shows a tSNE plot illustrating that the ICU day 1 COVID-19+ patient metabolome was distinct and easily separable from age- and sex-matched healthy control subjects. In fact, classification accuracy was 100% when comparing the 2 metabolomes. We then identified the top 8 metabolites underlying these differences between cohorts and are shown in Table 14 with their associated % importance. In the COVID-19+ cohort, relative to the healthy control subjects, kynurenine increased 5.1-fold while arginine decreased 0.5-fold, sarcosine decreased 0.6-fold and lysophosphatidylcholines (LysoPCs) all decreased 0.3-fold on average. The least number of metabolites that were required to maintain a 100% classification accuracy between cohorts was then determined, with only arginine (cutoff ≤52.8 μM) and kynurenine (cutoff≥3.1 μM) required. The excellent predictive ability of an arginine/kynurenine ratio for discriminating a COVID-19 patient from a healthy control subject (cutoff ≤15.7) is shown with ROC analysis in FIG. 1B (AUC 1.00; P=0.0002).

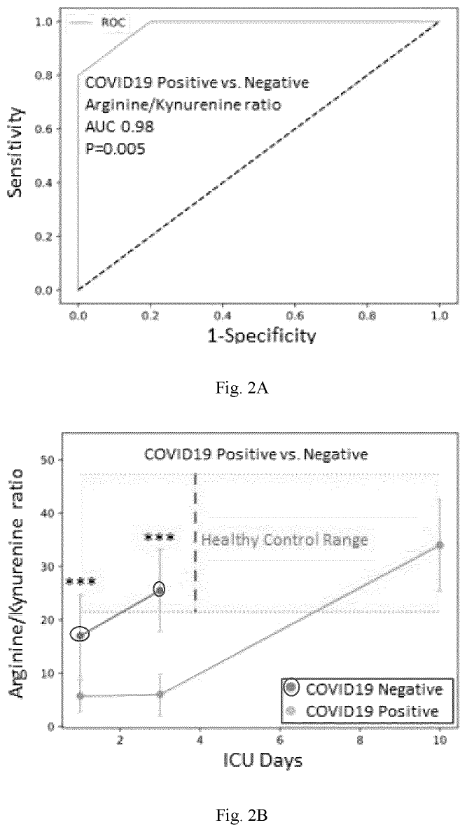

A comparison of COVID-19+ and COVID-19− patient cohorts revealed distinct metabolomes. Feature classification again identified kynurenine as one of the leading metabolites underlying the differences between COVID-19+ and COVID-19− cohorts (Table 15). We then determined that an arginine/kynurenine ratio again showed an excellent discriminative ability to determine COVID-19 status on ICU day 1 (5 cutoff <11.6) via ROC analyses (AUC 0.98; P=0.005; FIG. 2A). FIG. 2B shows an arginine/kynurenine ratio time plot for the COVID-19+ and COVID-19− patients over 10 ICU days. The cohorts' ratios were significantly different on ICU days 1 and 3 (P=0.005).

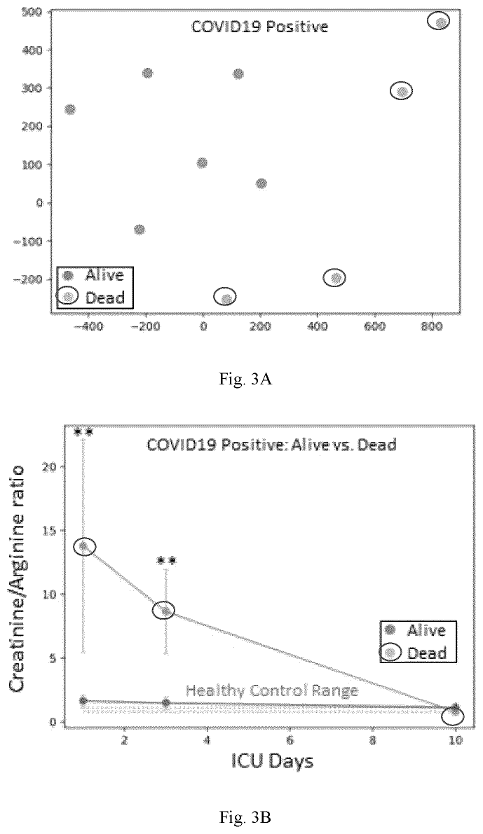

FIG. 3A shows a tSNE plot for COVID-19+ patients that either survived or died, and demonstrates that the outcomes were distinct and separable. To optimize outcome prediction in COVID-19+ patients, the number of metabolites were narrowed using feature selection (Table 16). Creatinine was the leading metabolite and it could predict death with 100% accuracy on both ICU days 1 (cutoff >126 μmol/l) and 3 (cutoff >174 μmol/l). To improve the variation in patient creatinine values, we then tested the ability of a creatinine/arginine ratio to predict death; the corresponding time plot is shown in FIG. 3B. Death could be predicted with 100% accuracy on both ICU days 1 (cutoff ≥3.4) and 3 (cutoff ≥3.7) as the creatinine/arginine ratios were significantly different between COVID-19 patients that lived or died at both time points (P=0.01). There were no deaths during the 10 ICU days.

162 metabolites in plasma obtained from ICU patients were measured, both COVID-19+ and COVID-19−, as well as age- and sex-matched healthy control subjects (see Table 13). Given the number of metabolites measured, the data was analyzed with machine learning. The data indicate the presence of a unique COVID-19 plasma metabolome dominated by changes in kynurenine, arginine, sarcosine and LysoPCs. Moreover, either creatinine alone or a creatinine/arginine ratio predicted ICU mortality with 100% accuracy.

Previous work in these same patients have determined a unique inflammatory profile characterized by elevated TNF and serine proteases (70), and a thrombotic profile associated with endothelial activation and glycocalyx degradation (71). We have also identified 6 novel protein immune biomarkers that predict COVID-19 associated death (72). Taken together with the data from this study, COVID-19 represents a severe illness with a unique pathophysiological signature, as well as a high mortality rate. Indeed, in our cohort of COVID-19 patients, ICU death was 40% with standardized ICU care.

The metabolites required for COVID-19 diagnosis (arginine, kynurenine, and/or arginine/kynurenine ratio) and outcome (either creatinine alone or creatinine/arginine ratio) can be easily measured using only mass spectrometry or immune assay, making their use as COVID-19 biomarkers affordable and easily available. Point-of-care analyses for these metabolites could be rapidly developed, such as a lateral flow immunochromatographic assay. Moreover, the results presented herein support the use of dietary supplementation of tryptophan, arginine, sarcosine and LysoPCs as adjunctive therapies for COVID-19.

COVID-19 status relied heavily on increased plasma kynurenine. The essential amino acid tryptophan is metabolized to elevate the energy producing cofactor nicotinamide adenosine dinucleotide, with kynurenine as the first stable intermediate to be formed (73). Increased degradation of tryptophan, with a consequential increase in kynurenine, occurs during an immune response and is driven by the release of interferon-gamma from activated T-cells. COVID-19 caused intense T-cell activation (74, 75) with an approximate 11-fold increase in plasma interferon-gamma in critically ill COVID-19 patients (70).

While plasma kynurenine effectively discriminated COVID-19+ patients from healthy control subjects, determination of COVID-19 status in ICU patients required further specificity that was optimally provided by an arginine/kynurenine ratio. Arginine, an amino acid precursor for nitric oxide, was significantly depressed in COVID-19+ patients. Arginine depletion is likely secondary to the intense requirement during COVID-19 for nitric oxide signaling and antimicrobial activity (76), as well as consumption by the enzyme arginase 1 (ARG1) that represents a macrophage immunoregulatory mechanism (77).

Sarcosine, an amino acid that helped discriminate COVID-19+ patients from healthy control subjects, was also significantly depressed. While not superior to the arginine/kynurenine ratio for diagnosing COVID-19 status, sarcosine sequestration may have a critical role in COVID-19 pathology. Sarcosine enhances the activity of antigen presenting cells (78) and activates autophagy (79), or the body's removal of damaged cells and their immunostimulatory debris. As a protective catabolic process during COVID-19, autophagy is critical to the antiviral response by direct elimination of virus, the presentation of viral antigens and the inhibition of excessive inflammation (80). Sarcosine levels decrease with age (79), and the elderly are most susceptible to COVID-19 morbidity and mortality.

Depressed plasma LysoPCs also helped discriminate COVID-19+ patients from healthy control subjects. The partial hydrolysis of phosphatidylcholines by phospholipase A2 produces LysoPCs, which are subsequently implicated in endothelial activation (81) and phagocytosis of cellular debris (82). Decreased plasma LysoPCs has been observed in sepsis (83), where LysoPCs may aid pathogen elimination, and therapeutic replacement has been suggested to improve sepsis outcome (84).

Acute renal dysfunction is strongly associated with high mortality in ICU patients (85). Plasma creatinine, a marker of renal dysfunction, was an excellent discriminator for COVID-19 patients that either lived or died. In our COVID-19+ cohort, 2 patients had chronic kidney disease and 2 patients required renal replacement therapy. The angiotensin-converting enzyme 2 receptor that is essential for SARS-CoV-2 uptake is highly expressed on tubule epithelial cells (86). Acute kidney injury is reported to occur in up to 37% of COVID-19 patients (87) and is secondary acute tubular injury from direct viral infection (88).

The data presented in this disclosure suggest that COVID-19 diagnosis (arginine/kynurenine ratio) and outcome (creatinine alone or creatinine/arginine ratio) can be easily determined with point-of-care measurements of kynurenine, arginine and creatinine, and that this rapid and affordable biomarker approach may be complimentary to the more expensive and time-consuming diagnostic tools currently employed (e.g. polymerase chain reaction and antigen immunoassay). Moreover, dietary supplementation of tryptophan, arginine, sarcosine and LysoPCs can aid COVID-19 outcome as therapies or adjunctive therapies.

In summary, we report a unique metabolome in COVID-19+ ICU patients, with identification of 3 metabolites that appear to be accurate diagnostic/prognostic biomarkers for future studies. Given the rapid spread of COVID-19 and the critical need for rapid and affordable diagnostics, our data may be invaluable for future testing. In addition, our exploratory data may be invaluable for guiding resource mobilization and/or goals of care discussion, but only after validation in larger COVID-19+ cohorts. Furthermore, patient stratification is critically important for future COVID-19 interventional trials.

TABLE 1
Subject demographics and clinical data.

| | Healthy | COVID19− | | COVID19+ | | |
|---|---|---|---|---|---|---|
| Variable | Controls | Patients | | Patients | | P-value |
| n | 10 | 10 | | 10 | | 1.000 |
| Age in years | 57.5 (52.8, 62.8) | 58.0 | (52.5, 63.0) | 61.0 | (54.8, 67.0) | 0.686 |
| Sex | 7F:3M | 7F:3M | | 7F:3M | | 1.000 |
| MODS | | 6.0 | (3.8, 8.0) | 4.0 | (2.5, 7.3) | 0.251 |
| SOFA | | 7.5 | (4.8, 11.0) | 4.5 | (2.8, 9.3) | 0.160 |
| Comorbidities | | | | | | |
| Hypertension | | 8 | (80) | 6 | (60) | 0.628 |
| Diabetes | | 4 | (40) | 3 | (30) | 1.000 |
| Chronic kidney disease | | 1 | (10) | 2 | (20) | 1.000 |
| Cancer | | 1 | (10) | 2 | (20) | 1.000 |
| COPD | | 1 | (10) | 0 | (0) | 1.000 |
| Baseline Medications | | | | | | |
| Antiplatelet agents | | 6 | (60) | 2 | (20) | 0.170 |
| Anticoagulants | | 1 | (10) | 0 | (0) | 1.000 |
| Baseline labs | | | | | | |
| WBC | | 15.3 | (11.1, 23.0) | 8.5 | (6.3, 16.1) | 0.064 |
| Neutrophils | | 12.2 | (8.1, 15.2) | 7.7 | (5.7, 13.3) | 0.197 |
| Lymphocytes | | 1.6 | (0.5, 2.3) | 0.7 | (0.6, 1.0) | 0.141 |
| Platelets | | 184 | (159, 245) | 206 | (109, 294) | 0.623 |
| Hemoglobin | | 130 | (104, 142) | 122 | (102, 136) | 0.364 |
| Creatinine | | 80 | (54, 147) | 107 | (55, 288) | 0.571 |
| Chest X-ray findings | | | | | | |
| Bilateral pneumonia | | 1 | (10) | 9 | (90) | 0.001* |
| Unilateral pneumonia | | 5 | (50) | 0 | (0) | 0.033* |
| Interstitial infiltrates | | 1 | (10) | 1 | (10) | 1.000 |
| Normal | | 3 | (30) | 0 | (0) | 0.211 |
| P:F ratio | | 172 | (132, 304) | 124 | (69, 202) | 0.153 |
| Sepsis diagnosis | | | | | | |
| Suspected | | 8 | (80) | 0 | (0) | 0.001* |
| Confirmed | | 2 | (20) | 10 | (100) | 0.001* |
| Interventions during study | | | | | | |
| Antibiotics | | 10 | (100) | 10 | (100) | 1.000 |
| Anti-virals | | 0 | (0) | 3 | (30) | 0.211 |
| Steroids | | 3 | (30) | 2 | (20) | 1.000 |
| Vasoactive medications | | 6 | (60) | 7 | (70) | 1.000 |
| VTE prophylaxis | | 10 | (100) | 10 | (100) | 1.000 |
| New antiplatelets | | 0 | (0) | 1 | (10) | 1.000 |
| New anticoagulation | | 2 | (20) | 1 | (10) | 1.000 |
| Renal replacement therapy | | 1 | (10) | 2 | (20) | 1.000 |
| High-flow nasal cannula | | 2 | (20) | 5 | (50) | 0.350 |
| Non-invasive MV | | 8 | (80) | 6 | (60) | 0.628 |
| Invasive MV | | 8 | (80) | 7 | (70) | 1.000 |
| Patient Outcome | | | | | | |
| New VTE/ischemic stroke | | 2 | (20) | 1 | (10) | 1.000 |
| Survived | | 10 | (100) | 6 | (60) | 0.087 |

Continuous data are presented as medians (IQRs), and categorical data are presented as n (%). MODS=Multiple Organ Dysfunction Score; SOFA=Sequential Organ Failure Assessment Score; COPD=Chronic Obstructive Pulmonary Disease; VTE=venous thromboembolism; MV=mechanical ventilation; VTE prophylaxis=number of patients receiving venous thromboembolism prophylaxis with regular or low molecular heparin; new antiplatelets=number of patients who were started on aspirin or clopidogrel during ICU stay; new anticoagulation=number of patients who were started on therapeutic anticoagulation with regular or low molecular heparin, or novel oral anticoagulants.

TABLE 2
Comparison of COVID19+ patients on ICU day
1 to healthy age- and sex-matched control patients.

| | Covid-19+ Patients | | Healthy Controls | | |
|---|---|---|---|---|---|
| Analyte | (n = 10) | | (n = 10) | | p |
| Elastase 2 | 40.2 | (19.0, 69.9) | 2.5 | (1.7, 3.2) | <0.001 |
| HSP70 | 208135 | (142253, 318061) | 26914 | (24981, 30710) | <0.001 |
| IL-1RA | 123.84 | (24.43, 1037.93) | 4.30 | (3.27, 4.77) | <0.001 |
| IL-6 | 88.13 | (39.35, 306.70) | 0.70 | (0.30, 1.56) | <0.001 |
| IL-8 | 8.84 | (5.67, 18.64) | 2.04 | (1.48, 2.71) | <0.001 |
| MCP-1 | 696.6 | (439.9, 1093.2) | 251.7 | (209.0, 336.6) | <0.001 |
| MIG | 10221 | (6285, 41017) | 1717 | (1126, 2294) | <0.001 |
| MMP8 | 2165 | (1379, 4173) | 255 | (128, 301) | <0.001 |
| Resistin | 39.15 | (30.26, 118.81) | 11.88 | (9.23, 14.09) | <0.001 |
| TNF | 194.4 | (124.3, 251.8) | 14.7 | (10.3, 25.5) | <0.001 |
| IL-10 | 44.26 | (17.80, 170.55) | 0 | (0, 4.95) | 0.001 |
| IL-18 | 141.4 | (84.6, 252.9) | 34.63 | (16.16, 44.92) | 0.001 |
| M-CSF | 184.2 | (127.6, 288.2) | 21.7 | (0, 38.0) | 0.001 |
| Granzyme B | 9.61 | (5.33, 23.12) | 2.27 | (1.65, 3.30) | 0.002 |
| Thrombospondin-1 | 1294 | (565, 2185) | 188 | (132, 460) | 0.002 |
| MIP-1β | 44.78 | (35.88, 58.30) | 31.09 | (24.13, 33.51) | 0.003 |
| MIMF2 | 71040 | (58159, 88142) | 120458 | (99649, 133271) | 0.004 |
| NGAL | 117.5 | (92.7, 506.7) | 74.90 | (62.92, 90.64) | 0.004 |
| IL-15 | 21.96 | (12.78, 49.86) | 6.69 | (4.79, 9.33) | 0.005 |
| IFN-γ | 1315 | (7.82, 144.80) | 1.69 | (0, 4.91) | 0.006 |

TABLE 3
Comparison of COVID19+ and COVID19− ICU patients (days 1-3).

| | ICU | COVID19+ Patients | | COVID19− Patients | | |
|---|---|---|---|---|---|---|
| Analyte | Day | (n = 10) | | (n = 10) | | P-value |
| TNF | 1 | 194.4 | (124.3, 251.8) | 21.0 | (6.4, 40.5) | <0.001* |
| | 2 | 141.2 | (103.7, 216.9) | 18.0 | (9.0, 39.8) | <0.001* |
| | 3 | 149.2 | (94.8, 206.6) | 16.4 | (2.0, 44.6) | 0.001* |
| Granzyme B | 1 | 9.61 | (5.33, 23.12) | 1.51 | (1.11, 2.98) | <0.001* |
| | 2 | 7.97 | (5.72, 13.63) | 1.26 | (0.74, 1.94) | <0.001* |
| | 3 | 8.31 | (5.02, 12.98) | 0.89 | (0.63, 1.80) | <0.001* |
| HSP70 | 1 | 208135 | (142253, 318061) | 106995 | (32750, 116705) | 0.002* |
| | 2 | 206109 | (134528, 308362) | 65791 | (40991, 116411) | 0.002* |
| | 3 | 236000 | (130712, 362185) | 62810 | (31960, 100732) | 0.001* |
| IL18 | 1 | 141.4 | (84.6, 252.9) | 33.8 | (17.5, 64.3) | 0.005* |
| | 2 | 140.8 | (86.8, 205.0) | 31.8 | (10.8, 65.7) | 0.001* |
| | 3 | 123.7 | (110.0, 189.8) | 48.2 | (21.5, 69.8) | <0.001* |
| IP10 | 1 | 3526 | (1407, 19503) | 165 | (41, 371) | 0.023 |
| | 2 | 2496 | (1081, 80080) | 94 | (54, 381) | 0.004* |
| | 3 | 4289 | (1108, 40564) | 149 | (65, 526) | <0.001* |
| Elastase 2 | 1 | 40.2 | (19.0, 69.9) | 21.6 | (15.6, 35.0) | 0.290 |
| | 2 | 71.78 | (33.92, 92.39) | 14.19 | (10.31, 23.04) | 0.002* |
| | 3 | 75.00 | (43.24, 121.40) | 24.98 | (10.65, 34.78) | 0.002* |
| IL10 | 1 | 44.26 | (17.80, 170.55) | 14.56 | (2.90, 28.17) | 0.010* |
| | 2 | 46.95 | (23.29, 156.33) | 4.21 | (0.14, 11.03) | 0.001* |
| | 3 | 34.26 | (23.16, 81.40) | 2.23 | (0, 9.42) | <0.001* |
| MIG | 1 | 10221 | (6285, 41017) | 2116 | (1268, 3975) | 0.001* |
| | 2 | 11180 | (4948, 37212) | 2684 | (1089, 4606) | 0.002* |
| | 3 | 12237 | (5795, 33781) | 1929 | (998, 6329) | 0.003* |
| M-CSF | 1 | 184.2 | (127.6, 288.2) | 13.1 | (0, 66.1) | 0.002* |
| | 2 | 162.1 | (91.2, 311.3) | 26.2 | (0, 102.0) | 0.004* |
| | 3 | 149.2 | (107.6, 284.1) | 16.0 | (0, 101.3) | 0.003* |
| IFNg | 1 | 18.15 | (7.82, 144.80) | 0 | (0, 1.25) | 0.001* |
| | 2 | 7.36 | (5.56, 26.24) | 0 | (0, 2.04) | 0.001* |
| | 3 | 7.95 | (5.42, 48.60) | 0.43 | (0, 3.35) | 0.005* |
| IL8 | 1 | 8.84 | (5.67, 18.64) | 2.51 | (1.13, 4.45) | 0.001* |
| | 2 | 6.51 | (2.96, 10.06) | 1.93 | (1.43, 3.32) | 0.005* |
| | 3 | 6.58 | (4.68, 12.21) | 1.67 | (0.65, 4.42) | 0.010* |
| MMP8 | 1 | 2165 | (1379, 4173) | 1666 | (910, 4323) | 0.364 |
| | 2 | 4308 | (2149, 7385) | 1219 | (521, 2753) | 0.008* |
| | 3 | 5745 | (2624, 10956) | 1812 | (460, 2503) | 0.002* |
| IL2 | 1 | 0.33 | (0, 1.87) | 0 | (0, 0) | 0.005* |
| | 2 | 0 | (0, 0.57) | 0 | (0, 0) | 0.031 |
| | 3 | 0.43 | (0, 0.75) | 0 | (0, 0) | 0.005* |
| IL15 | 1 | 21.96 | (12.78, 49.86) | 6.86 | (2.69, 11.97) | 0.008* |
| | 2 | 16.40 | (9.18, 31.76) | 8.06 | (3.17, 13.17) | 0.016 |
| | 3 | 17.10 | (10.37, 44.19) | 5.49 | (2.86, 12.69) | 0.010* |
| IL-1RA | 1 | 123.84 | (24.43, 1037.93) | 12.18 | (5.69, 29.66) | 0.019 |
| | 2 | 36.55 | (22.41, 622.70) | 5.72 | (3.26, 51.79) | 0.019 |
| | 3 | 55.44 | (21.86, 321.47) | 6.67 | (2.27, 16.22) | 0.003* |
| MMP1 | 1 | 1065 | (414, 2330) | 804 | (474, 1349) | 0.326 |
| | 2 | 1424 | (828, 3111) | 670 | (301, 1115) | 0.034 |
| | 3 | 1679 | (800, 3382) | 704 | (490, 1211) | 0.007* |
| MCP-1 | 1 | 696.6 | (439.9, 1093.2) | 356.7 | (215.4, 481.7) | 0.007* |
| | 2 | 548.0 | (453.3, 938.6) | 327.1 | (193.2, 530.0) | 0.023 |
| | 3 | 767.6 | (498.3, 1032.4) | 237.6 | (118.6, 748.4) | 0.041 |

Only analytes with statistically significant data on one or more days are shown. Data are presented as medians (IQRs) with analyte concentration in pg/ml (*p<0.01). Analytes are ordered by top 6 as found through machine learning analysis, then analytes with all 3 days significant most to least by day 3, then analytes significant on days 2 and 3 only most to least by day 3, then analytes significant on days 1 and 3 only most to least by day 3, then analytes most to least significant on day 3 only, then analytes significant on day 1 only.

TABLE 4
Summary of receiver operating characteristic curve
(ROC) analyses for predicting Death in COVID10.
All plasma analytes were measured on ICU Day 1.

| Variable | AUC | 95% CI | P-value |
|---|---|---|---|
| HSP70 | 1.00 | 1.00-1.00 | 0.002* |
| IL-1RA | 1.00 | 1.00-1.00 | 0.002* |
| IL10 | 1.00 | 1.00-1.00 | 0.002* |
| MIG | 1.00 | 1.00-1.00 | 0.002* |
| M-CSF | 0.98 | 0.94-1.00 | 0.003* |
| IL6 | 0.97 | 0.89-1.00 | 0.005* |
| IFNg | 0.95 | 0.86-1.00 | 0.006* |
| IL8 | 0.95 | 0.86-1.00 | 0.006* |
| TNFα | 0.95 | 0.85-1.00 | 0.006* |
| MCP-1 | 0.94 | 0.83-1.00 | 0.008* |
| P-selectin | 0.94 | 0.82-1.00 | 0.008* |
| MMP10 | 0.91 | 0.77-1.00 | 0.014 |
| MIP-1β | 0.88 | 0.70-1.00 | 0.023 |
| Elastase 2 | 0.86 | 0.66-1.00 | 0.030 |
| IL15 | 0.86 | 0.68-1.00 | 0.030 |
| MMP8 | 0.86 | 0.67-1.00 | 0.030 |
| MGAL | 0.86 | 0.66-1.00 | 0.030 |
| Resistin | 0.86 | 0.63-1.00 | 0.030 |
| IL2 | 0.85 | 0.57-1.00 | 0.033 |
| IL3 | 0.82 | 0.54-1.00 | 0.053 |
| Chondroitin | 0.81 | 0.62-1.00 | 0.059 |
| MMP2 | 0.81 | 0.58-1.00 | 0.059 |
| Lactoferrin | 0.80 | 0.48-1.00 | 0.073 |
| IL12(p40) | 0.78 | 0.57-0.99 | 0.089 |
| IL18 | 0.78 | 0.51-1.00 | 0.089 |
| MMP7 | 0.78 | 0.51-1.00 | 0.089 |
| vWF | 0.78 | 0.49-1.00 | 0.089 |
| Heparan | 0.77 | 0.53-1.00 | 0.098 |
| MIP-1α | 0.77 | 0.47-1.00 | 0.098 |
| MMP3 | 0.75 | 0.46-1.00 | 0.131 |
| Eotaxin | 0.73 | 0.46-1.00 | 0.156 |
| Granzyme B | 0.73 | 0.49-0.98 | 0.156 |
| Protein C | 0.73 | 0.48-0.99 | 0.156 |
| MMP13 | 0.73 | 0.51-0.95 | 0.171 |
| IL12(p70) | 0.71 | 0.41-1.00 | 0.202 |
| G-CSF | 0.70 | 0.34-1.00 | 0.219 |
| IL17F | 0.70 | 0.46-0.94 | 0.219 |
| IFNα2 | 0.70 | 0.47-0.92 | 0.238 |
| IL5 | 0.69 | 0.46-0.92 | 0.257 |
| Syndecan-1 | 0.69 | 0.36-1.00 | 0.257 |
| TNFβ | 0.69 | 0.36-1.00 | 0.257 |
| IP10 | 0.68 | 0.28-1.00 | 0.277 |
| MMP9 | 0.67 | 0.33-1.00 | 0.299 |
| Hyaluronic acid | 0.66 | 0.34-0.97 | 0.345 |
| MMP1 | 0.66 | 0.36-0.95 | 0.345 |
| Thrombospondin-1 | 0.66 | 0.40-0.91 | 0.345 |
| EGF | 0.64 | 0.32-0.96 | 0.395 |
| RANTES | 0.64 | 0.35-0.93 | 0.395 |
| IL7 | 0.63 | 0.30-0.95 | 0.450 |
| MMP12 | 0.63 | 0.24-1.00 | 0.450 |
| IL1α | 0.62 | 0.34-0.90 | 0.479 |
| PDGF-AA | 0.61 | 0.35-0.87 | 0.508 |
| GM-CSF | 0.60 | 0.25-0.95 | 0.539 |
| IL1β | 0.60 | 0.30-0.90 | 0.539 |
| IL17A | 0.60 | 0.29-0.91 | 0.539 |
| IL4 | 0.59 | 0.28-0.91 | 0.571 |
| PDGF-ABZBB | 0.59 | 0.24-0.95 | 0.571 |
| ADAMTS13 | 0.58 | 0.30-0.86 | 0.637 |
| IL22 | 0.56 | 0.23-0.90 | 0.705 |
| VEGFA | 0.56 | 0.25-0.87 | 0.705 |
| IL17E/IL25 | 0.53 | 0.24-0.82 | 0.850 |
| IL13 | 0.52 | 0.18-0.85 | 0.925 |

TABLE 5
ICU Day 1 analytes predict COVID19 patients versus healthy controls.

| Num | Assay | Unipro ID | Function |
|---|---|---|---|
| 1. | TYMP | P19971 | Thymidine phosphorylase also called platelet- |
| | | | derived endothelial cell growth factor is an |
| | | | intracellular protein whose major source is |
| | | | platelets. It may be involved in platelet |
| | | | activation and its secreted metabolites may |
| | | | potentiate thrombosis. |
| 2. | CXCL10 | P02778 | Induced by IFNγ, produced by endothelial |
| | | | cells, monocytes, fibroblasts and |
| | | | keratinocytes. Agonist for CXCR3 which is |
| | | | expressed on some T, B, and NK cells. |
| | | | Promotes Th1 recruitment induces T cell |
| | | | adherence to endothelial cells, |
| | | | chemoattractant for monocytes, T cells, NKs. |
| 3. | C1QA | P02745 | Complement C1q subunit A is one of 3 |
| | | | subunits making up C1q, part of the classical |
| | | | complement system. |
| 4. | AGR2 | O95994 | Anterior gradient protein 2 homolog is a |
| | | | member of the protein disulfide isomerase |
| | | | family normally located in the endoplasmic |
| | | | reticulum of intestinal cells, as well as the |
| | | | lung, stomach, colon and prostate; tissues |
| | | | with mucus secreting or endocrine functions. |
| 5. | IL-18R1 | Q13478 | One of the heterodimers of the IL-18 receptor |
| | | | complex, it is a type I transmembrane protein. |
| | | | Expressed on NKs, T cells especially |
| | | | activated Th1 cells; it induces IFNγ in concert |
| | | | with IL-12 receptor. |
| 6. | CDON | Q4KMG0 | Cell adhesion molecule-related, down- |
| | | | regulated by oncogenes is a transmembrane |
| | | | glycoprotein that acts as a cell adhesion |
| | | | molecule and binds members of the hedgehog |
| | | | family. It seems to normally be involved in |
| | | | development and proliferating cells. |
| 7. | DDX58 | O95786 | Retinoic acid-inducible gene 1, also called |
| | | | DEAD box protein 58, is a transmembrane |
| | | | pattern recognition receptor that recognizes |
| | | | viral replicative intermediates in the cytosol |
| | | | during RNA virus infections. It is expressed |
| | | | in endothelial cells and activates the type 1 |
| | | | interferon response. |
| 8. | CLEC6A | Q6EIG7 | C-type lectin domain family 6 member A, |
| | | | also called dectin-2 is a transmembrane |
| | | | pattern-recognition receptor highly expressed |
| | | | on macrophages as well as monocytes, |
| | | | Kupffer, Langerhans and some dendritic |
| | | | cells. It binds surface polysaccharides of |
| | | | pathogens and ultimately causes cytokine |
| | | | production to direct a Th17 response. |
| 9. | CLM-6 | Q08708 | CMRF35-like molecule 6 also called |
| | | | leukocyte mono-immunoglobulin-like |
| | | | receptor 8 (LMIR8) and CD300c is a |
| | | | transmembrane receptor expressed on almost |
| | | | all leukocytes and plasmacytoid dendritic |
| | | | cells it recognizes phosphatidylethanolamine |
| | | | on apoptotic cells. |
| 10. | PXN | P49023 | Paxillin is a cytosolic scaffolding protein |
| | | | involved in focal adhesions and integrin- |
| | | | mediated signal transduction. |
| 11. | LAG3 | P18627 | Lymphocyte activation gene 3 is a |
| | | | transmembrane receptor expressed on several |
| | | | types of T cells that regulates their function. |
| 12. | APLP1 | P51693 | Amyloid-like protein 1 is expressed |
| | | | exclusively in the central nervous system and |
| | | | is a cytosolic protein thought to be involved |
| | | | in cell-cell contacts in synapses. |
| 13. | LIF-R | P42702 | Leukemia inhibitory factor receptor is a |
| | | | transmembrane protein that along with gp130 |
| | | | forms receptor for LIF, a member of the IL-6 |
| | | | family. LIF-R is expressed in several organs |
| | | | as well as monocytes and macrophages. |
| 14. | B4GALT1 | P15291 | Beta-1,4-galactosyltransferase 1 is a trans- |
| | | | golgi membrane protein that transfers a sugar |
| | | | nucleotide to acceptors. When on the plasma |
| | | | membrane, it functions as a cell-adhesion |
| | | | molecule involved in cell-cell and cell-matrix |
| | | | interaction. |
| 15. | ASGR1 | P07306 | 1 of two subunits of the asialoglycoprotein |
| | | | receptor, which is mainly expressed in liver, |
| | | | that facilitates uptake of desialylated |
| | | | glycoproteins It is a member of the C-type |
| | | | lectin family of receptors and can clear |
| | | | hyposialylated vonWillebrand factor from |
| | | | plasma. |
| 16. | CRIM1 | Q9NZV1 | Cysteine-rich motor neuron 1 is a |
| | | | transmembrane receptor that regulates growth |
| | | | factor signaling in a number of organs during |
| | | | organogenesis. |
| 17. | CD300E | Q496F6 | Also called CLM-2, it is a transmembrane |
| | | | receptor expressed on the surface of |
| | | | monocytes and circulating myeloid dendritic |
| | | | cells. It appears to be involved in inducing |
| | | | cytokine release, reactive oxygen species |
| | | | production. |
| 18. | CDKN1A | P38936 | Cyclin-dependent kinase inhibitor 1 also |
| | | | called p21 and CIP1 is a nuclear and |
| | | | cytoplasmic protein which can arrest the cell |
| | | | cycle due to DNA damage and is anti- |
| | | | apoptotic. |
| 19. | CXCL11 | O14625 | A ligand for CXCR3 which is expressed on |
| | | | some T, B, and NK cells. It promotes Th1 |
| | | | recruitment/chemotaxis. |
| 20. | IL6 | P05231 | A key pro-inflammatory cytokine it can |
| | | | directly stimulate cells through its membrane- |
| | | | bound receptor on hepatocytes, neutrophils, |
| | | | monocytes, and some lymphocytes. In |
| | | | concert with its soluble receptor, it can also |
| | | | stimulate a wide-variety of cells that |
| | | | expresses gp130. |

CLM—CMRF35-like molecule
CXCR—CXC receptor
IFNγ—interferon gamma
IL—interleukin
NK—natural killer cell
Th1—type 1 T-helper cell

TABLE 6
ICU Day 1 analytes that predict COVID19 status.

| Num | Assay | Unipro ID | Function |
|---|---|---|---|
| 1. | DDX58 | O95786 | Retinoic acid-inducible gene 1, also called DEAD box |
| | | | protein 58, is a transmembrane pattern recognition |
| | | | receptor that recognizes viral replicative intermediates |
| | | | in the cytosol during RNA virus infections. It is |
| | | | expressed in endothelial cells and activates the type 1 |
| | | | interferon response. |
| 2. | RRM2B | Q7LG56 | Ribonucleoside-diphosphate reductase subunit M2 B |
| | | | also called p53R2, is a nuclear protein thought to be |
| | | | involved in DNA repair after damage. |
| 3. | IRF9 | Q00978 | Interferon regulatory factor 9 is a nuclear transcription |
| | | | factor that is part of a transcription complex that |
| | | | responds to type 1 interferon signaling. |
| 4. | NPM1 | P06748 | Nucleophosmin is a nucleolar protein found in |
| | | | proliferating cells, it has functions in mitotic spindle |
| | | | assembly, ribosome synthesis, DNA repair, |
| | | | embryogenesis and chromatin remodeling. It can bind |
| | | | HEXIM1 (see entry 10 below). |
| 5. | MCP-3 | P80098 | Monocyte chemotactic protein 3, also called C-C motif |
| | | | chemokine 7 (CCL7), is the ligand for CCR1, CCR2, |
| | | | CCR3 and CCR5. It attracts monocytes, T cells, NKs |
| | | | immature DCs, basophils and eosinophils. |
| 6. | Gal-9 | O00182 | Galectin-9 is a pattern recognition receptor that binds β- |
| | | | galactosides. Its expressed on various immune cells, |
| | | | especially T cells, and expression is increased in |
| | | | response to various stimuli such as mitogen, TLR |
| | | | activation, pro-inflammatory cytokines and viral |
| | | | infection. |
| 7. | NADK | O95544 | nicotinamide adenine dinucleotide (NAD) kinase is a |
| | | | ubiquitously expressed cytosolic protein that |
| | | | phosphorylates NAD. |
| 8. | BRK1 | Q8WUW1 | Brick1 also called Hspc300, is a cytoskeleton- |
| | | | associated component of the Wave protein complex. |
| 9. | PFDN2 | Q9UHV9 | Prefoldin subunit 2 is a cytosolic protein, part of the |
| | | | hexameric prefoldin complex that captures unfolded |
| | | | proteins and transfers them to a chaperonin. |
| 10. | HEXIM1 | O94992 | Hexamethylene bis-acetamide-inducible protein 1 is a |
| | | | nuclear transcriptional regulator. It can bind to |
| | | | nucleophosmin (see entry 4 above) and hypoxia- |
| | | | inducible factor α; may be recruited by NF-κB for |
| | | | transcription of inflammation-responsive genes. |
| 11. | TCN2 | P20062 | Transcobalamin II is a serum protein that binds vitamin |
| | | | B12 for transport. It is synthesized in the intestinal |
| | | | mucosa, liver, seminal vesicles, fibroblasts, bone |
| | | | marrow, and macrophages. |
| 12. | BLM | Q13867 | Bleomycin hydrolase is a cytosolic cysteine |
| | hydrolase | | aminopeptidase which breaks down homocysteine. |
| 13. | KRT19 | P08727 | Keratin 19 also called cytokeratin 19 is a cytoskeletal |
| | | | protein expressed in laminated epithelium. |
| 14. | FUS | P35637 | Fused in sarcoma is a ubiquitously expressed nuclear |
| | | | RNA and DNA binding protein, though it also is found |
| | | | in the cytoplasm. |
| 15. | RCOR1 | Q9UKL0 | REST corepressor 1 is a nuclear protein that is part of |
| | | | protein complexes that modify chromatin to repress |
| | | | gene expression. |
| 16. | PSME1 | Q06323 | Proteasome activator complex subunit 1, also called |
| | | | PA28α, is a subunit of PA(proteasome activator)28 that |
| | | | binds to and activates the 20S proteasome. It is a |
| | | | cytosolic protein expressed in most tissues and induced |
| | | | by IFNγ. |
| 17. | CXCL11 | O14625 | A ligand for CXCR3 which is expressed on some T, B, |
| | | | and NK cells. It promotes Th1 recruitment/chemotaxis. |
| 18. | CLSPN | Q9HAW4 | Claspin is a nuclear protein that associates with DNA |
| | | | replication stalled due to DNA damage. It's expression |
| | | | is tightly regulated during the cell cycle with high levels |
| | | | in late S phase and G2. |
| 19. | S100A11 | P31949 | A cytosolic calcium-binding protein of the S100 family, |
| | | | it is involved in growth arrest in contact inhibition. It is |
| | | | expressed in a wide variety of cells and is secreted by |
| | | | an unconventional pathway. It is involved in cell-cell |
| | | | contacts and can promote cell migration in response to |
| | | | hypoxia-induced mitogenic factor. |
| 20. | CDON | Q4KMG0 | Cell adhesion molecule-related, down-regulated by |
| | | | oncogenes is a transmembrane glycoprotein that acts as |
| | | | a cell adhesion molecule and binds members of the |
| | | | hedgehog family. It seems to normally be involved in |
| | | | development and proliferating cells. |

CXCR—C-X-C motif receptor
DC—dendritic cell
DEAD—Asp-Glu-Ala-Asp
IFNγ—interferon gamma
NF—κB—nuclear factor kappa B
NK—natural killer cell
REST—repressor for element-1 silencing transcription
Th#—type # T-helper cell
TLR—toll-like receptor

TABLE 7
ICU Day 1 analytes that predict outcome.

| Num | Assay | Unipro ID | Function |
|---|---|---|---|
| 1. | CXCL9 | Q07325 | A chemokine produced by airway epithelial cells in |
| | | | response to infection, also induced by IFNγ and in |
| | | | endothelial cells by TNFα. An agonist for CXCR3 on T |
| | | | cells and natural killer cells. Promotes NK, Th1, monocyte, |
| | | | DC, neutrophil, and eosinophil recruitment. |
| 2. | ICOSLG | O75144 | Inducible costimulator ligand is expressed by B cells, |
| | | | monocytes, DCs T cells and endothelial cells; TNFα is |
| | | | required for induction. It activates the inducible |
| | | | costimulator in the thymus and on activated T cells. |
| 3. | CLM-1 | Q8TDQ1 | CMRF35-like molecule 1 is a receptor for |
| | | | phosphatidylserine presented on the outer membrane |
| | | | surface of apoptotic cells, that promotes macrophage and |
| | | | inhibits DC efferocytosis. |
| 4. | IL12RB1 | P42701 | One of the two subunits that compose the IL-12 receptor, its |
| | | | signaling pathway activates STAT4. Expressed primarily |
| | | | on activated T cells and NK cells, less so on dendritic cells |
| | | | and some B-cells. This subunit is also shared with the IL-23 |
| | | | receptor. |
| 5. | CD83 | Q01151 | Expressed on B and T cells, monocytes, DCs, microglia and |
| | | | neutrophils, and has soluble and membrane-bound forms. |
| | | | Membrane-bound CD83 is essential for CD4⁺ T cell |
| | | | development and inhibiting autoimmunity, soluble CD83 |
| | | | induces regulatory mechanisms for tolerance. |
| 6. | CA12 | O43570 | Carbonic anhydrase 12 is membrane-associated |
| | | | glycoprotein that catalyzes the reversible hydration of |
| | | | carbon dioxide. CA12 is up-regulated by hypoxia, at least in |
| | | | tumor environments and CA activity is associated with |
| | | | sleep apnea-related hypoxemia. |
| 7. | FLRT2 | O43155 | Fibronectin leucine rich transmembrane protein 2 was first |
| | | | discovered in a screen for extracellular matrix proteins and |
| | | | participates in homotypic cell-cell adhesion and with |
| | | | fibroblast growth factor receptor. |
| 8. | ROR1 | Q01973 | A transmembrane receptor tyrosine kinase that is activated |
| | | | by Wnt family ligands and is mainly thought to be involved |
| | | | in organ/tissue genesis during development. Though recent |
| | | | evidence suggests it may be involved in pro-inflammatory |
| | | | p65 activation, at least in cancer. |
| 9. | IL32 | P24001 | Expressed by PBMCs, epithelial cells and NKs, it up- |
| | | | regulates other pro-inflammatory cytokines and has several |
| | | | isoforms. Airway epithelial cell production is increased by |
| | | | viral infections and oxididative stress. |
| 10. | NCS1 | P62166 | Neuronal calcium sensor 1 is a cytosolic protein involved in |
| | | | several cellular functions through binding partners and |
| | | | intracellular Ca²⁺ regulation. It is highly expressed in |
| | | | neurons, but is not neuron-specific. |
| 11. | S100A11 | P31949 | A cytosolic calcium-binding protein of the S100 family, it is |
| | | | involved in growth arrest in contact inhibition. It is |
| | | | expressed in a wide variety of cells and is secreted by an |
| | | | unconventional pathway. It is involved in cell-cell contacts |
| | | | and can promote cell migration in response to hypoxia- |
| | | | induced mitogenic factor. |
| 12. | ANGPTL7 | O43827 | Angiopoietin-like protein 7 is an orphan ligand, but appears |
| | | | to be involved in hematopoietic stem cell regulation and |
| | | | self-renewal. Its serum concentration is higher in obese |
| | | | subjects compared to non-obese controls and can be |
| | | | lowered with exercise. |
| 13. | CLMP | Q9H6B4 | coxsackievirus and adenovirus receptor-like membrane |
| | | | protein is a transmembrane glycoprotein involved in |
| | | | homophilic cell-cell adhesion and is expressed in a wide |
| | | | variety of tissues. |
| 14. | IGF1R | P08069 | A tyrosine kinase receptor expressed on T and B cells, |
| | | | macrophages, NEC cells and granulocytes where its ligands, |
| | | | insulin-like growth factor 1 and 2, causes various effects |
| | | | such as proliferation, cytokine production and |
| | | | priming/activation. |
| 15. | TOP2B | Q02880 | DNA topoisomerase II beta is expressed in a wide variety of |
| | | | tissues and throughout the cell cycle. Mostly found in the |
| | | | cell's nucleus, it is one of the enzymes that catalyzes |
| | | | topological changes in DNA. |
| 16. | FAM3B | P58499 | Also called pancreatic derived factor (PANDER), it is |
| | | | highly expressed in pancreatic islets and high serum levels |
| | | | are associated with the progression of metabolic syndrome |
| | | | and type 2 diabetes. |
| 17. | IL10.1 | P22301 | An important anti-inflammatory cytokine, expressed in |
| | | | virtually all immune cells except plasmacytoid DCs, to limit |
| | | | immune responses and prevent host damage. |
| 18. | IL10 | P22301 | See “IL10.1” above. |
| 19. | THY 1 | P04216 | Thymocyte differentiation antigen 1 is a glycoprotein |
| | | | expressed on the outer surface of many cell types including |
| | | | fibroblasts, T cells and activated endothelial cells and has a |
| | | | soluble form. Its function is cell and tissue-dependent, but is |
| | | | pro-fibrotic in pulmonary fibroblasts in pulmonary fibrosis. |
| 20. | PVRL4 | Q96NY8 | Poliovirus receptor-related protein 4 also called nectin-4 is a |
| | | | cell-cell adhesion molecule in aherens junctions, |
| | | | overexpressed in several cancers. |
| 21. | OPTC | Q9UBM4 | Opticin is an extracellular matrix protein associated with |
| | | | collagen in the vitreous humor where it binds heparan and |
| | | | chondroitin sulfate. It is an anti-angiogenic factor in retinas. |

CXCR—CXC receptor
DC—dendritic cell
IFNγ—interferon gamma
IL—interleukin
IFNγ—interferon gamma
NK—natural killer cell
PBMC—peripheral blood monocyte cell
STAT—signal transducer and activator of transcription
Th#—type # T helper cell
TNFα—tumor necrosis factor alpha

TABLE 8
ICU Day 3 analytes that predict outcome.

| Num | Assay | Uniprot ID | Function |
|---|---|---|---|
| 1. | IL12RB1 | P42701 | One of the two subunits that form the IL-12 receptor. |
| | | | Expressed primarily on activated T cells and NK cells, less |
| | | | so on dendritic cells and some B-cells. This subunit is also |
| | | | shared with the IL-23 receptor. |
| 2. | CLM-1 | Q8TDQ1 | CMRF35-like molecule 1 also called CD300f, is a receptor |
| | | | for phosphatidylserine presented on the outer membrane of |
| | | | apoptotic cells that promotes macrophage and inhibits DC |
| | | | efferocytosis. |
| 3. | CXCL9 | Q07325 | A chemokine produced by airway epithelial cells in |
| | | | response to infection, also induced by IFNγ and in |
| | | | endothelial cells by TNF. An agonist for CXCR3 on T |
| | | | cells and natural killer cells. Promotes NK, Th1, |
| | | | monocyte, DC, neutrophil, and eosinophil recruitment. |
| 4. | FAM3B | P58499 | Family with sequence similarity 3, also called pancreatic |
| | | | derived factor (PANDER), is a cytokine-like protein that is |
| | | | highly expressed in pancreatic islets and high serum levels |
| | | | are associated with the progression of metabolic syndrome |
| | | | and type 2 diabetes. |
| 5. | OPTC | Q9UBM4 | Opticin is an extracellular matrix protein associated with |
| | | | collagen in the vitreous humor where it binds heparan and |
| | | | chondroitin sulfate. It is an anti-angiogenic factor in |
| | | | retinas. |
| 6. | THY 1 | P04216 | Thymocyte differentiation antigen 1 is a glycoprotein |
| | | | expressed on the outer surface of many cell types |
| | | | including fibroblasts, T cells and activated endothelial |
| | | | cells and has a soluble form. Its function is cell and tissue- |
| | | | dependent, but is pro-fibrotic in pulmonary fibroblasts in |
| | | | pulmonary fibrosis. |
| 7. | ICOSLG | O75144 | Inducible costimulator ligand is a transmembrane protein |
| | | | expressed by B cells, monocytes, DCs T cells and |
| | | | endothelial cells; TNFα is required for induction. It |
| | | | activates the inducible costimulator in the thymus and on |
| | | | activated T cells. |
| 8. | IGF1R | P08069 | A tyrosine kinase receptor expressed on T and B cells, |
| | | | macrophages, NK cells and granulocytes where its ligands, |
| | | | insulin-like growth factor 1 and 2, causes various effects |
| | | | such as proliferation, cytokine production and |
| | | | priming/activation. |
| 9. | IL10.1 | P22301 | An important anti-inflammatory cytokine, expressed in |
| | | | virtually all immune cells except plasmacytoid DCs, it |
| | | | works to limit immune responses and prevent host |
| | | | damage. |
| 10. | CLMP | Q9H6B4 | coxsackievirus and adenovirus receptor-like membrane |
| | | | protein is a transmembrane glycoprotein involved in |
| | | | homophilic cell-cell adhesion and is expressed in a wide |
| | | | variety of tissues. |
| 11. | IL10 | P22301 | See “IL10.1” above. |
| 12. | CD83 | Q01151 | Expressed on B and T cells, monocytes, DCs, microglia |
| | | | and neutrophils, and has soluble and membrane-bound |
| | | | forms. Membrane-bound CD83 is essential for CD4⁺ T |
| | | | cell development and inhibiting autoimmunity, soluble |
| | | | CD83 induces regulatory mechanisms for tolerance. |
| 13. | ROR1 | Q01973 | A transmembrane receptor tyrosine kinase that is activated |
| | | | by Wnt family ligands and is mainly thought to be |
| | | | involved in organ/tissue genesis during development. |
| | | | Though recent evidence suggests it may be involved in |
| | | | pro-inflammatory p65 activation, at least in cancer. |
| 14. | PVRL4 | Q96NY8 | Poliovirus receptor-related protein 4 also called nectin-4 is |
| | | | a cell-cell adhesion molecule in aherens junctions, |
| | | | overexpressed in several cancers. |
| 15. | IL32 | P24001 | Expressed by PBMCs, epithelial cells and NKs, it up- |
| | | | regulates other pro-inflammatory cytokines and has several |
| | | | isoforms. Airway epithelial cell production is increased by |
| | | | viral infections and oxididative stress. |
| 16. | CA12 | O43570 | Carbonic anhydrase 12 is membrane-associated |
| | | | glycoprotein that catalyzes the reversible hydration of |
| | | | carbon dioxide. CA12 is up-regulated by hypoxia, at least |
| | | | in tumor environments and CA activity is associated with |
| | | | sleep apnea-related hypoxemia. |
| 17. | NCS1 | P62166 | Neuronal calcium sensor 1 is a cytosolic protein involved |
| | | | in several cellular functions through binding partners and |
| | | | intracellular Ca²⁺ regulation. It is highly expressed in |
| | | | neurons, but is not neuron-specific. |
| 18. | FLRT2 | O43155 | Fibronectin leucine rich transmembrane protein 2 was first |
| | | | discovered in a screen for extracellular matrix proteins and |
| | | | participates in homotypic cell-cell adhesion as well as |
| | | | fibroblast growth factor receptor signaling. |
| 19. | S100A11 | P31949 | A cytosolic calcium-binding protein of the S100 family, it |
| | | | is involved in growth arrest in contact inhibition. It is |
| | | | expressed in a wide variety of cells and is secreted by an |
| | | | unconventional pathway. It is involved in cell-cell contacts |
| | | | and can promote cell migration in response to hypoxia- |
| | | | induced mitogenic factor. |
| 20. | TOP2B | Q02880 | DNA topoisomerase II beta is expressed in a wide variety |
| | | | of tissues and throughout the cell cycle. Mostly found in |
| | | | the cell's nucleus, it is one of the enzymes that catalyzes |
| | | | topological changes in DNA. |
| 21. | ANGPTL7 | O43827 | Angiopoietin-like protein 7 is an orphan ligand, but |
| | | | appears to be involved in hematopoietic stem cell |
| | | | regulation and self-renewal. Its serum concentration is |
| | | | higher in obese subjects compared to non-obese controls |
| | | | and can be lowered with exercise. |

CD—cluster of differentiation
CXCL—CXC ligand
CXCR—CXC receptor
DC—dendritic cell
IFNγ—interferon gamma
IL—interleukin
IFNγ—interferon gamma
NK—natural killer cell
PBMC—peripheral blood monocyte cell
STAT—signal transducer and activator of transcription
Th#—type # T helper cell
TNFα—tumor necrosis factor alpha

TABLE 9
Comparison of COVID-19+ patients on ICU day
1 to healthy age- and sex-matched control patients.

| Variable | Healthy Controls | | COVID-19+ Patients | | P-value |
|---|---|---|---|---|---|
| Thrombosis factors | | | | | |
| ADAMTS13 (ng/ml) | 788 | (609, 1075) | 633 | (463, 794) | 0.151^{a} |
| Protein C (μg/ml) | 12.1 | (3.2, 15.5) | 6.2 | (0.9, 10.5) | 0.131^{b} |
| vWF (ng/ml) | 1536 | (927, 3320) | 7018 | (4916, 22821) | <0.001 |
| Endothelial injury | | | | | |
| sP-selectin (ng/ml) | 20.7 | (16.2, 43.4) | 33.3 | (16.2, 44.5) | 0.623^{c} |
| Heparan Sulfate (ng/ml) | 2.4 | (2.0, 4.5) | 3.4 | (2.7, 9.8) | 0.450^{d} |
| Chondroitin Sulfate (pg/ml) | 1.6 | (1.0, 5.0) | 7.0 | (5.1, 11.0) | 0.004 |
| Hyaluronic acid (ng/ml) | 54.6 | (29.3, 73.4) | 307.7 | (39.8, 633.7) | 0.226^{e} |
| Syndecan-1 (ng/ml) | 76.0 | (26.3, 97.6) | 181.9 | (103.6, 313.3) | 0.004 |

Data are presented as medians (IQRs).
vWF = von Willebrand factor.
Based on measured values, the following number of patients per cohort would be required to potentially reach statistical significance (80% power):
^{a}40;
^{b}30;
^{c}76136;
^{d}121;
^{e}13.

TABLE 10
Comparison of COVID-19− and COVID-19+ patients on ICU days 1-3.

| | ICU | COVID-19− | | COVID-19+ | | |
|---|---|---|---|---|---|---|
| Variable | Day | Patients | | Patients | | P-value |
| Thrombosis factors | | | | | | |
| ADAMST13 (ng/ml) | 1 | 713 | (357, 1005) | 633 | (463, 794) | 0.880^{a} |
| | 2 | 626 | (343, 885) | 637 | (581, 862) | 0.597^{b} |
| | 3 | 661 | (397, 968) | 616 | (526, 733) | 0.940^{c} |
| Protein C (μg/ml) | 1 | 10.2 | (2.2, 11.9) | 6.2 | (0.9, 10.5) | 0.257^{d} |
| | 2 | 8.8 | (1.3, 12.1) | 8.1 | (0.6, 9.8) | 0.406^{e} |
| | 3 | 11.2 | (1.2, 14.3) | 6.0 | (0.9, 12.1) | 0.326^{f} |
| vWF (ng/ml) | 1 | 7203 | (3718, 14500) | 7018 | (4916, 22821) | 0.199^{g} |
| | 2 | 7319 | (4067, 13450) | 11833 | (4422, 21872) | 0.406^{h} |
| | 3 | 10027 | (5747, 15794) | 7128 | (4410, 21614) | 0.650ⁱ |
| Endothelial injury | | | | | | |
| sP-selectin (ng/ml) | 1 | 23.7 | (16.0, 34.1) | 33.3 | (16.2, 44.5) | 0.406^{j} |
| | 2 | 30.1 | (16.4, 42.0) | 25.7 | (17.9, 29.3) | 0.496^{k} |
| | 3 | 22.0 | (16.5, 31.6) | 47.0 | (25.0, 57.8) | 0.028 |
| Heparan Sulfate (ng/ml) | 1 | 2.8 | (1.5, 4.2) | 3.4 | (2.7, 9,8) | 0.186^{l} |
| | 2 | 1.8 | (1.2, 4.1) | 4.3 | (2.4, 8.2) | 0.049 |
| | 3 | 1.9 | (1.2, 2.6) | 3.2 | (1.9, 6.0) | 0.070^{m} |
| Chondroitin Sulfate (pg/ml) | 1 | 4.4 | (3.4, 10.7) | 7.0 | (5.1, 11.0) | 0.082ⁿ |
| | 2 | 5.2 | (2.3, 13.7) | 5.9 | (4.9, 6.5) | 0.762^{o} |
| | 3 | 5.2 | (2.8, 7.0) | 7.0 | (5.1, 9.6) | 0.226^{p} |
| Hyaluronic acid (ng/ml) | 1 | 71.7 | (28.7, 153.2) | 307.7 | (39.8, 633.7) | 0.315^{q} |
| | 2 | 107.1 | (39.4, 192.1) | 340.5 | (58.9, 745.5) | 0.199^{r} |
| | 3 | 78.5 | (37.9, 168.1) | 354.9 | (67.6, 874.2) | 0.010 |
| Syndecan-1 (ng/ml) | 1 | 46.9 | (1.9, 89.0) | 181.9 | (103.6, 313.3) | 0.010 |
| | 2 | 53.6 | (13.5, 101.4) | 296.7 | (142.2, 743.4) | 0.005 |
| | 3 | 54.0 | (3.2, 98.8) | 413.5 | (139.7, 755.9) | 0.004 |

Data are presented as medians (IQRs).
vWF = von Willebrand factor.
Based on measured values, the following number of patients per cohort would be required to potentially reach statistical significance (80% power):
^{a}1111;
^{b}1972;
^{c}3568;
^{d}184;
^{e}1266;
^{f}73;
^{g}115;
^{h}60;
ⁱ3236;
^{j}167;
^{k}110;
^{l}32;
^{m}22;
ⁿ29195;
^{o}277;
^{p}42;
^{q}24;
^{r}20.

TABLE 11
Comparison of Healthy Controls, COVID-19− and COVID-19+ patients on ICU day 3.

| | Healthy | COVID-19− | | COVID-19+ | | | |
|---|---|---|---|---|---|---|---|
| | Controls | ICU Day 3 | | ICU Day 3 | | P-value | Post-Hoc Analysis |
| sP-selectin | 20.7 (16.2, 43.4) | 22.0 | (16.5, 31.6) | 47.0 | (25.0, 57.8) | 0.080 | N/A |
| Hyaluronic | 54.6 (29.3, 73.4) | 78.5 | (37.9, 168.1) | 354.9 | (67.6, 874.2) | 0.003 | HC vs CoV+ = 0.004 |
| acid | | | | | | | CoV− vs CoV+ = 0.012 |
| Syndecan-1 | 76.0 (26.3, 97.6) | 54.0 | (3.2, 98.8) | 413.5 | (139.7, 755.9) | 0.002 | HC vs CoV+ = 0.004 |
| | | | | | | | CoV− vs CoV+ = 0.003 |

TABLE 12

| | | Healthy Controls: | Case |
|---|---|---|---|
| Plasma Analyte | Units | CI: 5%, 95% | Patient |
| 1. MMP7 | pg/ml | 3356, 4424 | 51788 |
| 2. IP-10 | pg/ml | 86, 242 | 1098 |
| 3. Resistin | pg/ml | 7.3, 11.1 | 41.8 |
| 4. IL-3 | pg/ml | 0.1, 2.4 | 7.3 |
| 5. Hyaluronic acid (EC) | ng/ml | 17.6, 40.4 | 119.2 |
| 6. Thrombospondin-1 | pg/ml | 620, 1275 | 3286 |
| 7. Elastase 2 | pg/ml | 2.1, 4.4 | 11.3 |
| 8. PDGF-AB/BB | pg/ml | 769, 2537 | 6390 |
| 9. MIG | pg/ml | 1205, 2684 | 6531 |
| 10. MCP-1 | pg/ml | 190.3, 269.5 | 529.4 |
| 11. MMP1 | pg/ml | 384, 709 | 1286 |
| 12. Lactoferrin | pg/ml | 338.3, 521.3 | 845.8 |
| 13. IL-IRA | pg/ml | 8.1, 73.3 | 112.0 |
| 14. IL-18 | pg/ml | 30.3, 64.6 | 98.3 |
| 15. IFNα2 | pg/ml | 13.2, 120.7 | 179.6 |
| 16. P-selectin (EC) | ng/ml | 16.3, 22.4 | 30.4 |
| 17. MIP-1β | pg/ml | 22.8, 72.0 | 89.16 |
| 18. Eotaxin | pg/ml | 49.4, 81.6 | 97.4 |
| 19. MMP8 | pg/ml | 288.4, 643.6 | 762.7 |
| 20. PDGF-AA | pg/ml | 84.4, 652.9 | 766.8 |
| 21. MMP10 | pg/ml | 385.2, 751.4 | 876.1 |
| 22. Heparin sulfate (EC) | ng/ml | 22.7, 294.5 | 20.6 |

TABLE 13
List of Metabolites

| | | | | |
|---|---|---|---|---|
| Creatinine | Sarcosine | LYSOC24:0 | C6:1 | Dimethylglycine |
| Glycine | Diacetylspermine | LYSOC26:1 | C6 | Ethanol |
| Alanine | Creatine | LYSOC26:0 | C5OH | Glycerol |
| Serine | Betaine | LYSOC28:1 | C5:1DC | Formate |
| Proline | Choline | LYSOC28:0 | C5DC | Hypoxanthine |
| Valine | Trimethylamine | 14:1SMOH | C8 | D-Mannose |
| | N-oxide | | | |
| Threonine | Methylhistidine | 16:1SM | C5MDC | L-Acetylcarnitine |
| Phenylethylamine | Homocysteine | 16:0SM | C9 | Oxoglutarate |
| Taurine | Lactic acid | 16:1SMOH | C7DC | Urea |
| Putrescine | beta- | 18:1SM | C10:2 | 3-Hydroxybutyric |
| | Hydroxybutyric | | | acid |
| | acid | | | |
| trans- | alpha- | PC32:2AA | C10:1 | 2- |
| Hydroxyproline | Ketoglutaric acid | | | hydroxyisovalerate |
| Leucine | Citric acid | 18:0SM | C10 | L-Alpha- |
| | | | | aminobutyric acid |
| Isoleucine | Butyric acid | 20:2SM | C12:1 | 3-Methyl-2- |
| | | | | oxovaleric acid |
| Asparagine | Propionic acid | PC36:0AE | C12 | Malonate |
| Aspartic acid | HPHPA | PC36:6AA | C14:2 | Ketoleucine |
| Glutamine | p- | PC36:0AA | C14:1 | 3- |
| | Hydroxyhippuric | | | Hydroxyisovaleric |
| | acid | | | acid |
| Glutamic acid | Succinic acid | 22:2SMOH | C14 | Isopropanol |
| Methionine | Fumaric acid | 22:1SMOH | C12DC | Acetone |
| Dopamine | Pyruvic acid | PC38:6AA | C14:2OH | Methanol |
| Histidine | Isobutyric acid | PC38:0AA | C14:1OH | Propylene glycol |
| Phenylalanine | Hippuric acid | PC40:6AE | C16:2 | Dimethyl sulfone |
| Methionine- | Methylmalonic | 24:1SMOH | C16:1 | |
| sulfoxide | acid | | | |
| Arginine | Homovanillic acid | PC40:6AA | C16 | |
| Acetyl-ornithine | Indole-3-acetic | PC40:2AA | C16:2OH | |
| | acid | | | |
| Citrulline | Uric acid | PC40:1AA | C16:1OH | |
| Serotonin | Glucose | C0 | C16OH | |
| Tyrosine | LYSOC14:0 | C2 | C18:2 | |
| Asymmetric | LYSOC16:1 | C3:1 | C18:1 | |
| dimethylarginine | | | | |
| Total | LYSOC16:0 | C3 | C18 | |
| dimethylarginine | | | | |
| Tryptophan | LYSOC17:0 | C4:1 | C18:1OH | |
| Kynurenine | LYSOC18:2 | C4 | 2- | |
| | | | Hydroxybutyrate | |
| Ornithine | LYSOC18:1 | C3OH | Acetic acid | |
| Lysine | LYSOC18:0 | C5:1 | Acetoacetate | |
| Spermidine | LYSOC20:4 | C5 | L-Carnitine | |
| Spermine | LYSOC20:3 | C4OH | Dimethylamine | |

TABLE 14
Feature classification demonstrating the top 8 plasma
metabolites that classify COVID-19+ status versus
healthy control subjects with their % association.

| | Metabolite | Importance (%) |
|---|---|---|
| 1. | Kynurenine | 10 |
| 2. | Arginine | 10 |
| 3. | Sarcosine | 10 |
| 4. | LYSOPC18:1 | 10 |
| 5. | LYSOPC20:4 | 10 |
| 6. | LYSOPC14:0 | 10 |
| 7. | LYSOPC17:0 | 10 |
| 8. | LYSOPC18:2 | 10 |

TABLE 15
Feature classification demonstrating the top 8 plasma
metabolites that classify COVID-19+ status
versus COVID-19− patients with their % association.

| | Metabolite | Importance (%) |
|---|---|---|
| 1. | Kynurenine | 10 |
| 2. | LysoPC17:0 | 10 |
| 3. | LysoPC20:3 | 10 |
| 4. | C5:1DC | 10 |
| 5. | C6:1 | 10 |
| 6. | Glycine | 8 |
| 7. | Threonine | 8 |
| 8. | Histidine | 6 |

TABLE 16
Feature classification demonstrating the top 8 plasma
metabolites that classify COVID-19+ ICU patient
outcome as alive or dead with their % association. Plasma
creatinine was 25 the leading outcome predictor metabolite.

| | Metabolite | Importance (%) |
|---|---|---|
| 1. | Creatinine | 20 |
| 2. | Creatine | 10 |
| 3. | C3OH | 10 |
| 4. | PC40:6AA | 10 |
| 5. | C5 | 10 |
| 6. | C6:1 | 10 |
| 7. | C3:1 | 10 |
| 8. | Methylmalonic acid | 10 |

TABLE 17
Feature classification demonstrating the top 20 inflammatory
analytes that classify COVID-19 status in ICU patients'
days 1-3 with their % association (FIG. 8).

| Num | Assay | Unipro Id | Importance |
|---|---|---|---|
| 1. | TYMP | P19971 | (0.036364) |
| 2. | CXCL10 | P02778 | (0.021818) |
| 3. | C1QA | P02745 | (0.020000) |
| 4. | AGR2 | O95994 | (0.020000) |
| 5. | IL-18R1 | Q13478 | (0.020000) |
| 6. | CDON | Q4KMG0 | (0.020000) |
| 7. | DDX58 | O95786 | (0.020000) |
| 8. | CLEC6A | Q6EIG7 | (0.019939) |
| 9. | CLM-6 | Q08708 | (0.016364) |
| 10. | PXN | P49023 | (0.016364) |
| 11. | LAG3 | P18627 | (0.016364) |
| 12. | APLP1 | P51693 | (0.014848) |
| 13. | LIF-R | P42702 | (0.014848) |
| 14. | B4GALT1 | P15291 | (0.013333) |
| 15. | ASGR1 | P07306 | (0.012468) |
| 16. | CRIM1 | Q9NZV1 | (0.011818) |
| 17. | CD300E | Q496F6 | (0.011818) |
| 18. | CDKN1A | P38936 | (0.011515) |
| 19. | CXCL11 | O14625 | (0.010000) |
| 20. | IL6 | P05231 | (0.010000) |

TABLE 18
Feature classification demonstrating the top 20 inflammatory
analytes that classify COVID-19 status in ICU patients'
days 1-3 with their % association (FIG. 9).

| Num | Assay | Uniprot ID | Importance |
|---|---|---|---|
| 1. | DDX58 | O95786 | (0.040045) |
| 2. | RRM2B | Q7LG56 | (0.022396) |
| 3. | IRF9 | Q00978 | (0.020178) |
| 4. | NPM1 | P06748 | (0.019699) |
| 5. | MCP-3 | P80098 | (0.019348) |
| 6. | Gal-9 | O00182 | (0.019016) |
| 7. | NADK | O95544 | (0.017407) |
| 8. | BRK1 | Q8WUW1 | (0.017145) |
| 9. | PFDN2 | Q9UHV9 | (0.017051) |
| 10. | HEXIM1 | O94992 | (0.016900) |
| 11. | TCN2 | P20062 | (0.016566) |
| 12. | BLM hydrolase | Q13867 | (0.015594) |
| 13. | KRT19 | P08727 | (0.014615) |
| 14. | FUS | P35637 | (0.014505) |
| 15. | RCOR1 | Q9UKL0 | (0.014103) |
| 16. | PSME1 | Q06323 | (0.013333) |
| 17. | CXCL11 | O14625 | (0.012911) |
| 18. | CLSPN | Q9HAW4 | (0.012748) |
| 19. | S100A11 | P31949 | (0.012333) |
| 20. | CDON | Q4KMG0 | (0.011295) |

TABLE 19
The top 21 proteins underlying the outcome
differences shown in FIG. 10A

| Num | Assay | Unipro Id | Importance |
|---|---|---|---|
| 1. | CXCL9 | Q07325 | (0.071033) |
| 2. | ICOSLG | O75144 | (0.068896) |
| 3. | CLM-1 | Q8TDQ1 | (0.052505) |
| 4. | IL12RB1 | P42701 | (0.052471) |
| 5. | CD83 | Q01151 | (0.049965) |
| 6. | CA12 | O43570 | (0.049572) |
| 7. | FLRT2 | O43155 | (0.049366) |
| 8. | ROR1 | Q01973 | (0.048488) |
| 9. | IL32 | P24001 | (0.048049) |
| 10. | NCS1 | P62166 | (0.047003) |
| 11. | S100A11 | P31949 | (0.045890) |
| 12. | ANGPTL7 | O43827 | (0.044741) |
| 13. | CLMP | Q9H6B4 | (0.044442) |
| 14. | IGF1R | P08069 | (0.043656) |
| 15. | TOP2B | Q02880 | (0.043410) |
| 16. | FAM3B | P58499 | (0.042812) |
| 17. | IL10.1 | P22301 | (0.041501) |
| 18. | IL10 | P22301 | (0.041058) |
| 19. | THY 1 | P04216 | (0.040373) |
| 20. | PVRL4 | Q96NY8 | (0.038997) |
| 21. | OPTC | Q9UBM4 | (0.035772) |

TABLE 20
Top 21 proteins underlying the outcome
differences shown in FIG. 10B.

| Num | Assay | Unipro Id | Importance |
|---|---|---|---|
| 1. | IL12RB1 | P42701 | (0.090633) |
| 2. | CLM-1 | Q8TDQ1 | (0.089183) |
| 3. | CXCL9 | Q07325 | (0.077083) |
| 4. | FAM3B | P58499 | (0.076017) |
| 5. | OPTC | Q9UBM4 | (0.071080) |
| 6. | THY 1 | P04216 | (0.070551) |
| 7. | ICOSLG | O75144 | (0.060209) |
| 8. | IGF1R | P08069 | (0.055931) |
| 9. | IL10.1 | P22301 | (0.054822) |
| 10. | CLMP | Q9H6B4 | (0.050275) |
| 11. | IL10 | P22301 | (0.049304) |
| 12. | CD83 | Q01151 | (0.046050) |
| 13. | ROR1 | Q01973 | (0.044522) |
| 14. | PVRL4 | Q96NY8 | (0.042428) |
| 15. | IL32 | P24001 | (0.036729) |
| 16. | CA12 | O43570 | (0.028727) |
| 17. | NCS1 | P62166 | (0.024985) |
| 18. | FLRT2 | O43155 | (0.015163) |
| 19. | S100A11 | P31949 | (0.006715) |
| 20. | TOP2B | Q02880 | (0.006509) |
| 21. | ANGPTL7 | O43827 | (0.003084) |

TABLE 21
Feature classification demonstrating the top 15 inflammatory
analytes that classify COVID-19 status in ICU patients'
days 1-3 with their % association (see FIG. 12).

| Rank | Analyte | % Association |
|---|---|---|
| 1 | TNF | 10.1 |
| 2 | Granzyme B | 7.8 |
| 3 | HSP70 | 7.6 |
| 4 | IL-18 | 6.4 |
| 5 | IP-10 | 4.2 |
| 6 | Elastase 2 | 3.9 |
| 7 | MIG | 3.2 |
| 8 | IL-8 | 3.2 |
| 9 | IL-17A | 3.2 |
| 10 | IFNα2 | 2.9 |
| 11 | M-CSF | 2.8 |
| 12 | IL-2 | 2.7 |
| 13 | IL-15 | 2.6 |
| 14 | IL-10 | 2.4 |
| 15 | IL-1β | 2.3 |

### REFERENCES

1. Bhatraju P K, Ghassemieh B J, Nichols M, Kim R, et al: Covid-19 in Critically Ill Patients in the Seattle Region—Case Series. N Engl J Med 2020
2. Zhou F, Yu T, Du R, Fan G, et al: Clinical course and risk factors for mortality of adult inpatients with COVID-19 in Wuhan, China: a retrospective cohort study. Lancet 2020; 395(10229):1054-1062
3. Wu C, Chen X, Cai Y, Xia J, et al: Risk Factors Associated With Acute Respiratory Distress Syndrome and Death in Patients With Coronavirus Disease 2019 Pneumonia in Wuhan, China. JAMA Intern Med 2020
4. Grasselli G, Zangrillo A, Zanella A, Antonelli M, et al: Baseline Characteristics and Outcomes of 1591 Patients Infected With SARS-CoV-2 Admitted to ICUs of the Lombardy Region, Italy. JAMA 2020
5. Auld S C, Caridi-Scheible M, Blum J M, Robichaux C, et al: ICU and Ventilator Mortality Among Critically Ill Adults With Coronavirus Disease 2019. Crit Care Med 2020.
6. Tisoncik J R, Korth M J, Simmons C P, Farrar J, Martin T R, Katze M G. Into the eye of the cytokine storm. Microbiol Mol Biol Rev 2012; 76:16-32.
7. Mehta P, McAuley D F, Brown M, et al. COVID-19: consider cytokine storm syndromes and immunosuppression. Lancet 2020; 395:1033-4.
8. Zhou F, Yu T, Du R, et al. Clinical course and risk factors for mortality of adult inpatients with COVID-19 in Wuhan, China: a retrospective cohort study. Lancet 2020; 395:1054-62.
9. Huang C, Wang Y, Li X, et al. Clinical features of patients infected with 2019 novel coronavirus in Wuhan, China. Lancet 2020; 395:497-506.
10. Feldmann M, Maini R N, Woody J N, et al. Trials of anti-tumour necrosis factor therapy for COVID-19 are urgently needed. Lancet 2020.
11. Ritchie A I, Singanayagam A. Immunosuppression for hyperinflammation in COVID-19: a double-edged sword? Lancet 2020; 395:1111.
12. Mokra D, Mikolka P, Kosutova P, Mokry J. Corticosteroids in Acute Lung Injury: The Dilemma Continues. Int J Mol Sci 2019; 20.
13. https://www.cdc.gov/coronavirus/2019-nCoV/hcp/clinical-criteria.html:
14. https://www.fda.gov/media/134922/download:
15. Priestap F, Kao R, Martin CM: External validation of a prognostic model for intensive care unit mortality: a retrospective study using the Ontario Critical Care Information System. Can J Anaesth 2020
16. Singer M, Deutschman C S, Seymour C W, Shankar-Hari M, et al: The Third International Consensus Definitions for Sepsis and Septic Shock (Sepsis-3). JAMA 2016; 315(8):801-810
17. Brisson A R, Matsui D, Rieder M J, Fraser D D: Translational research in pediatrics: tissue sampling and biobanking. Pediatrics 2012; 129(1):153-162
18. Gillio-Meina C, Cepinskas G, Cecchini E L, Fraser DD: Translational research in pediatrics II: blood collection, processing, shipping, and storage. Pediatrics 2013; 131(4):754-766
19. Program NHRP: POLICY: Guidelines for Limits of Blood Drawn for Research Purposes in the Clinical Center M95-9 (rev) 2009; June 5
20. van der Maaten L, Hinton G: Visualizing data using t-SNE. J Mach Learn Res 2008; 9(11):2579-2605.
21. Assarsson E, Lundberg M, Holmquist G, Bjorkesten J, et al: Homogenous 96-plex PEA immunoassay exhibiting high sensitivity, specificity, and excellent scalability. PLoS One 2014; 9(4):e95192
22. Lundberg M, Eriksson A, Tran B, Assarsson E, et al: Homogeneous antibody-based proximity extension assays provide sensitive and specific detection of low-abundant proteins in human blood. Nucleic Acids Res 2011; 39(15):e102.
23. Fraser D D, Cepinskas G, Slessarev M, Martin C, et al: Inflammation profiling of critically ill COVID19 patients. Crit Care Explor 2020; 2:e0144.
24. Gillio-Meina C, Cepinskas G, Cecchini E L, Fraser DD. Translational research in pediatrics II: blood collection, processing, shipping, and storage. Pediatrics 2013; 131:754-66.
25. Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell 2020.
26. Cullen S P, Brunet M, Martin S J. Granzymes in cancer and immunity. Cell Death Differ 2010; 17:616-23.
27. Lauw F N, Simpson A J, Hack C E, et al. Soluble granzymes are released during human endotoxemia and in patients with severe infection due to gram-negative bacteria. J Infect Dis 2000; 182:206-13.
28. Wensink A C, Hack C E, Bovenschen N. Granzymes regulate proinflammatory cytokine responses. J Immunol 2015; 194:491-7.
29. Buzza M S, Zamurs L, Sun J, et al. Extracellular matrix remodeling by human granzyme B via cleavage of vitronectin, fibronectin, and laminin. J Biol Chem 2005; 280:23549-58.
30. Omoto Y, Yamanaka K, Tokime K, et al. Granzyme B is a novel interleukin-18 converting enzyme. J Dermatol Sci 2010; 59:129-35.
31. Akeda T, Yamanaka K, Tsuda K, Omoto Y, Gabazza E C, Mizutani H. CD8+ T cell granzyme B activates keratinocyte endogenous IL-18. Arch Dermatol Res 2014; 306:125-30.
32. Morel J C, Park C C, Woods J M, Koch A E. A novel role for interleukin-18 in adhesion molecule induction through NF kappa B and phosphatidylinositol (PI) 3-kinase-dependent signal transduction pathways. J Biol Chem 2001; 276:37069-75.
33. Pinsky M R, Vincent J L, Deviere J, Alegre M, Kahn R J, Dupont E. Serum cytokine levels in human septic shock. Relation to multiple-system organ failure and mortality. Chest 1993; 103:565-75.
34. Abraham E, Anzueto A, Gutierrez G, et al. Double-blind randomised controlled trial of monoclonal antibody to human tumour necrosis factor in treatment of septic shock. NORASEPT II Study Group. Lancet 1998; 351:929-33.
35. Wang L, Chung J, Gill S E, Mehta S: Quantification of adherens junction disruption and contiguous paracellular protein leak in human lung endothelial cells under septic conditions. Microcirculation 2019; 26(3):e12528
36. Wang L, Mehta S, Ahmed Y, Wallace S, et al: Differential Mechanisms of Septic Human Pulmonary Microvascular Endothelial Cell Barrier Dysfunction Depending on the Presence of Neutrophils. Front Immunol 2018; 9:1743
37. Faul F, Erdfelder E, Buchner A, Lang A G: Statistical power analyses using G*Power 3.1: tests for correlation and regression analyses. Behav Res Methods 2009; 41(4):1149-1160
38. van der Maaten L, Hinton G: Visualizing data using t-SNE. J Mach Learn Res 2008;
39. Zhou F, Yu T, Du R, Fan G, et al: Clinical course and risk factors for mortality of adult inpatients with COVID-19 in Wuhan, China: a retrospective cohort study. Lancet 2020; 395(10229): 1054-1062
40. Wu C, Chen X, Cai Y, Xia J, et al: Risk Factors Associated With Acute Respiratory Distress Syndrome and Death in Patients With Coronavirus Disease 2019 Pneumonia in Wuhan, China. JAMA Intern Med 2020
41. Bhatraju P K, Ghassemieh B J, Nichols M, Kim R, et al: Covid-19 in Critically Ill Patients in the Seattle Region—Case Series. N Engl J Med 2020
42. Grasselli G, Zangrillo A, Zanella A, Antonelli M, et al: Baseline Characteristics and Outcomes of 1591 Patients Infected With SARS-CoV-2 Admitted to ICUs of the Lombardy Region, Italy. JAMA 2020
43. Fraser D D, Cepinskas G, Slessarev M, Martin C, et al: Inflammation profiling of critically ill COVID19 patients. Crit Care Explor 2020; In press.
44. Teng Y H, Aquino R S, Park P W: Molecular functions of syndecan-1 in disease. Matrix Biol 2012; 31(1):3-16
45. Fitzgerald M L, Wang Z, Park P W, Murphy G, et al: Shedding of syndecan-1 and -4 ectodomains is regulated by multiple signaling pathways and mediated by a TIMP-3-sensitive metalloproteinase. J Cell Biol 2000; 148(4):811-824
46. Zhang Y, Xiao M, Zhang S, Xia P, et al: Coagulopathy and Antiphospholipid Antibodies in Patients with Covid-19. N Engl J Med 2020; 382(17):e38
47. Ji H L, Zhao R, Matalon S, Matthay M A: Elevated Plasmin(ogen) as a Common Risk Factor for COVID-19 Susceptibility. Physiol Rev 2020; 100(3):1065-1075
48. Hofstra J J, Haitsma J J, Juffermans N P, Levi M, et al: The role of bronchoalveolar hemostasis in the pathogenesis of acute lung injury. Semin Thromb Hemost 2008; 34(5):475-484
49. Tang N, Bai H, Chen X, Gong J, et al: Anticoagulant treatment is associated with decreased mortality in severe coronavirus disease 2019 patients with coagulopathy. J Thromb Haemost 2020; 18(5): 1094-1099
50. Mehta P, McAuley D F, Brown M, Sanchez E, et al: COVID-19: consider cytokine storm syndromes and immunosuppression. Lancet 2020; 395(10229):1033-1034
51. Reindel R, Baker S C, Kim K Y, Rowley C A, Shulman S T, Orenstein J M, et al. Integrins alpha4 and alphaM, collagen1A1, and matrix metalloproteinase 7 are upregulated in acute Kawasaki disease vasculopathy. Pediatr Res. 2013; 73(3):332-6.
52. Swee M, Wilson C L, Wang Y, McGuire J K, Parks W C. Matrix metalloproteinase-7 (matrilysin) controls neutrophil egress by generating chemokine gradients. J Leukoc Biol. 2008; 83(6):1404-12.
53. Ko T M, Kuo H C, Chang J S, Chen S P, Liu Y M, Chen H W, et al. CXCL10/IP-10 is a biomarker and mediator for Kawasaki disease. Circ Res. 2015; 116(5):876-83.
54. Kemmotsu Y, Saji T, Kusunoki N, Tanaka N, Nishimura C, Ishiguro A, et al. Serum adipokine profiles in Kawasaki disease. Mod Rheumatol. 2012; 22(1):66-72.
55. Nozue H, Imai H, Saitoh H, Aoki T, Ichikawa K, Kamoda T. Serum resistin concentrations in children with Kawasaki disease. Inflamm Res. 2010; 59(11):915-20.
56. Korpelainen E I, Gamble J R, Vadas M A, Lopez A F. IL-3 receptor expression, regulation and function in cells of the vasculature. Immunol Cell Biol. 1996; 74(1):1-7.
57. Kolarova H, Ambruzova B, Svihalkova Sindlerova L, Klinke A, Kubala L. Modulation of endothelial glycocalyx structure under inflammatory conditions. Mediators Inflamm. 2014; 2014:694312.
58. Ohnishi Y, Yasudo H, Suzuki Y, Furuta T, Matsuguma C, Azuma Y, et al. Circulating endothelial glycocalyx components as a predictive marker of coronary artery lesions in Kawasaki disease. Int J Cardiol. 2019; 292:236-40.
59. Schrijver I T, Kemperman H, Roest M, Kesecioglu J, de Lange D W. Soluble P-selectin as a Biomarker for Infection and Survival in Patients With a Systemic Inflammatory Response Syndrome on the Intensive Care Unit. Biomark Insights. 2017; 12:1177271916684823.
60. Gupta M, Noel G J, Schaefer M, Friedman D, Bussel J, Johann-Liang R. Cytokine modulation with immune gamma-globulin in peripheral blood of normal children and its implications in Kawasaki disease treatment. J Clin Immunol. 2001; 21(3):193-9.
61. Andreasen A S, Krabbe K S, Krogh-Madsen R, Taudorf S, Pedersen B K, Moller K. Human endotoxemia as a model of systemic inflammation. Curr Med Chem. 2008; 15(17):1697-705.
62. Chen, Peter et al. “MMP7 shedding of syndecan-1 facilitates re-epithelialization by affecting alpha(2)beta(1) integrin activation.” PloS one vol. 4.8 e6565. 10 Aug. 2009, doi:10.1371/journal.pone.0006565.
63. Foroutan A, Fitzsimmons C, Mandal R, Piri-Moghadam H, et al: The Bovine Metabolome. Metabolites 2020; 10(6)
64. Foroutan A, Guo A C, Vazquez-Fresno R, Lipfert M, et al: Chemical Composition of Commercial Cow's Milk. J Agric Food Chem 2019; 67(17):4897-4914
65. Psychogios N, Hau D D, Peng J, Guo A C, et al: The human serum metabolome. PLoS One 2011; 6(2):e16957
66. Saude E J, Slupsky C M, Sykes B D: Optimization of NMR analysis of biological fluids for quantitative accuracy. Metabolomics 2006; 2(3):113-123
67. Ravanbakhsh S, Liu P, Bjorndahl T C, Mandal R, et al: Accurate, fully-automated NMR spectral profiling for metabolomics. PLoS One 2015; 10(5):e0124219
68. van der Maaten L, Hinton G: Visualizing data using t-SNE. J Mach Learn Res 2008; 9(11):2579-2605
69. Tang C, Garreau D, von Luxburg U: When do random forests fail? Proceedings of the 32nd International Conference on Neural Information Processing Systems 2018; December 2018:2987-2997
70. Fraser D D, Cepinskas G, Slessarev M, Martin C, et al: Inflammation Profiling of Critically Ill Coronavirus Disease 2019 Patients. Critical Care Explorations 2020; 2(6):e0144
71. Fraser D D, Patterson E K, Slessarev M, Gill S E, et al: Endothelial Injury and Glycocalyx Degradation in Critically Ill Coronavirus Disease 2019 Patients: Implications for Microvascular Platelet Aggregation. Critical Care Explorations 2020; In press.
72. Fraser D D, Cepinskas G, Patterson E K, Slessarev M, et al: Novel Outcome Biomarkers Identified with Targeted Proteomic Analyses of Plasma from Critically Ill Coronavirus Disease 2019 Patients. Critical Care Explorations 2020; In press
73. Chen Y, Guillemin G J: Kynurenine pathway metabolites in humans: disease and healthy States. Int J Tryptophan Res 2009; 2:1-19
74. De Biasi S, Meschiari M, Gibellini L, Bellinazzi C, et al: Marked T cell activation, senescence, exhaustion and skewing towards TH17 in patients with COVID-19 pneumonia. Nat Commun 2020; 11(1):3434
75. Chen Z, John Wherry E: T cell responses in patients with COVID-19. Nat Rev Immunol 2020
76. Akerstrom S, Mousavi-Jazi M, Klingstrom J, Leijon M, et al: Nitric oxide inhibits the replication cycle of severe acute respiratory syndrome coronavirus. J Virol 2005; 79(3):1966-1969
77. Mondanelli G, Iacono A, Allegrucci M, Puccetti P, et al: Immunoregulatory Interplay Between Arginine and Tryptophan Metabolism in Health and Disease. Front Immunol 2019; 10:1565
78 5. Dastmalchi F, Karachi A, Yang C, Azari H, et al: Sarcosine promotes trafficking of dendritic cells and improves efficacy of anti-tumor dendritic cell vaccines via CXC chemokine family signaling. J Immunother Cancer 2019; 7(1):321
79. Walters R O, Arias E, Diaz A, Burgos E S, et al: Sarcosine Is Uniquely Modulated by Aging and Dietary Restriction in Rodents and Humans. Cell Rep 2018; 25(3):663-676 e666
80. Carmona-Gutierrez D, Bauer M A, Zimmermann A, Kainz K, et al: Digesting the crisis: autophagy and coronaviruses. Microb Cell 2020; 7(5):119-128
81. Li X, Fang P, Li Y, Kuo Y M, et al: Mitochondrial Reactive Oxygen Species Mediate Lysophosphatidylcholine-Induced Endothelial Cell Activation. Arterioscler Thromb Vasc Biol 2016; 36(6):1090-1100
82. Lauber K, Bohn E, Krober S M, Xiao Y J, et al: Apoptotic cells induce migration of phagocytes via caspase-3-mediated release of a lipid attraction signal. Cell 2003; 113(6):717-730
83. Mecatti G C, Fernandes Messias M C, Sant'Anna Paiola R M, Figueiredo Angolini C F, et al: Lipidomic Profiling of Plasma and Erythrocytes From Septic Patients Reveals Potential Biomarker Candidates. Biomark Insights 2018; 13:1177271918765137
84. Cunningham T J, Yao L, Lucena A: Product inhibition of secreted phospholipase A2 may explain lysophosphatidylcholines' unexpected therapeutic properties. J Inflamm (Loud) 2008; 5:17
85. Clermont G, Acker C G, Angus D C, Sirio C A, et al: Renal failure in the ICU: comparison of the impact of acute renal failure and end-stage renal disease on ICU outcomes. Kidney Int 2002; 62(3):986-996
86. Wysocki J, Lores E, Ye M, Soler M J, et al: Kidney and Lung ACE2 Expression after an ACE Inhibitor or an Ang II Receptor Blocker: Implications for COVID-19. J Am Soc Nephrol 2020
87. Hirsch J S, Ng J H, Ross D W, Sharma P, et al: Acute kidney injury in patients hospitalized with COVID-19. Kidney Int 2020; 98(1):209-218
88. Farkash E A, Wilson A M, Jentzen J M: Ultrastructural Evidence for Direct Renal Infection with SARS-CoV-2. J Am Soc Nephrol 2020; 31(8):1683-1687

Through the embodiments that are illustrated and described, the currently contemplated best mode of making and using the invention is described. Without further elaboration, it is believed that one of ordinary skill in the art can, based on the description presented herein, utilize the present invention to the full extent. All publications cited herein are incorporated by reference.

Although the description above contains many specificities, these should not be construed as limiting the scope of the invention, but as merely providing illustrations of some of the presently embodiments of this invention.

1. A method of diagnosing and treating a COVID-19 infection in a subject comprising: (a) providing a subject identified as having an increased level of a biomarker relative to a known reference levels of the biomarker, the biomarker being one or more of granzyme B, tumor necrosis factor (TNF), heat shock protein 70 (HSP70), interleukin-18 (IL-18), interferon-gamma-inducible protein 10 (IP-10), elastase 2, and syndecan-1, and (b) treating the subject for COVID-19 with an agent that reduces the level of the biomarker.

2. The method of claim 1, wherein the biomarker is one or more of granzyme B, TNF, HSP70 and IL18.

3. (canceled)

4. The method of claim 1, wherein the method further comprises obtaining a sample from the subject during the subject's treatment for COVID-19, wherein decrease in the levels of the biomarker in the recovery sample relative to the levels obtained in the test sample is indicative of a normalization of the subject.

5. The method of claim 1, to wherein the normal reference levels of the biomarker are derived from healthy subjects or from COVID-19 negative subjects.

6-11. (canceled)

12. The method of claim 1, wherein the method comprises administering to the subject a protease inhibitor.

13. The method of claim 12, wherein the protease inhibitor is a soybean-based protease inhibitor.

14-18. (canceled)

19. The method of claim 1, wherein the biomarker includes syndecan-1, and wherein step (b) comprises administering to the patient an agent that reduces the levels of syndecan-1 degradation product in plasma or protects/restores vascular syndecan-1.

20. The method of claim 19, wherein the agent that reduces the levels of syndecan-1 degradation product in plasma or protects/restores vascular syndecan-1 is sulodexide.

21. The method of claim 19, wherein the agent that reduces the levels of syndecan-1 degradation product in plasma or protects/restores vascular syndecan-1 is an inhibitor of a granzyme B or an inhibitor of elastase 2 or an inhibitor of metalloproteinase (MMP) activity.

22. The method of claim 19, wherein the agent that reduces the levels of syndecan-1 degradation product in plasma or protects/restores vascular syndecan-1 is a protease inhibitor.

23-24. (canceled)

25. The method of claim 19, wherein the agent that reduces the levels of syndecan-1 degradation product in plasma or protects/restores vascular syndecan-1 is an inhibitor of a syndican-1 sheddase.

26. The method of claim 21, wherein the inhibitor of MMP activity is sphingosine-1-phosphate or a protease inhibitor.

27. The method of claim 21, wherein the MMP is MMP2, MMP7 or MMP9.

28. The method of claim 19, wherein the agent that reduces the levels of syndecan-1 degradation product in plasma or protects/restores vascular syndecan-1 is a heparinase inhibitor.

29. The method of claim 19, wherein the agent that reduces the levels of syndecan-1 degradation product in plasma or protects/restores vascular syndecan-1 is heparin/heparan.

30. The method of claim 19, wherein the agent that reduces the levels of syndecan-1 degradation product in plasma is low molecular weight heparin.

31. The method of claim 19, wherein the patient is further treated with at least one additional agent, wherein the addition agent is one or more of an agent which blocks platelet aggregation or an anticoagulant or an agent which enhances thrombolysis.

32-36. (canceled)

37. A method of determining disease severity for a COVID-19 a patient, including mortality, the method comprising: (a) obtaining a test sample from the patient, (b) performing one or more assays configured to detect one or more disease severity biomarkers in the test sample, (c) obtaining the levels of the one or more disease severity biomarkers in the test sample, (d) comparing levels of the one or more disease severity biomarkers in the test sample with a normal control reference value of said one or more disease severity biomarkers, wherein an increase in the level of the one or more disease severity biomarkers in the test sample relative to the normal control reference value of said one or more disease severity biomarkers is indicative of the patient having a severe case of COVID-19, wherein the one or more biomarkers are syndican-1, hyaluronic acid (HA), chondroitin sulfate ADAMTS13, heparin sulfate, Protein C, sP-selectin, von Willebrand factor (vWF), HSP70, IL-1RA, IL 10, MIG, CLM-1, IL12RB1, CD83, FAM3B, IGF1R and OPTC, and (e) when the patient is diagnosed as having the severe case of COVID-19, then treating the patient with immunosuppression therapy with steroids, intravenous immunoglobulin, and/or selective cytokine blockade.

38-46. (canceled)

47. The method of claim 37, wherein step (c) comprises measuring the patient's concentration of the one or more disease severity biomarkers in absolute weight or absolute moles per volume; and wherein the normal control reference is a threshold level of concentration in absolute weight or absolute moles per volume corresponding to the one or more biomarkers and step (d) comprises comparing the measured concentration to the threshold level of concentration in absolute weight or absolute moles per volume corresponding to the measured one or more markers, wherein levels above the threshold level for the one or more biomarkers concentration in absolute weight or absolute moles per volume indicate that the patient is at risk of severe disease including mortality.

48. A method of diagnosing COVID-19 in a subject, the method comprising measuring levels of one or more metabolites listed in Table 13 in a sample taken from the subject, wherein a diagnosis of COVID-19 positive is indicated when the levels of said one or more metabolites are statistically different from known normal levels of said one or more metabolites, wherein when the subject is indicated as being COVID-19 positive, the subject is treated with tryptophan, arginine, sarcosine and/or LysoPCs or any combinations thereof.

49-84. (canceled)
